(19) United States
(12) Patent Application Publication
(10) Pub. No.: US 2026/0097087 A1
(43) Pub. Date: Apr. 9, 2026

(54) RECOMBINANT FUSION POLYPEPTIDES FOR SECRETING SOLUBLE, HETEROLOGOUS CARGO

(71) Applicant: NOVOME BIOTECHNOLOGIES, INC., South San Francisco, CA (US)

(72) Inventors: Weston R. Whitaker, Boston, MA (US); William C. DeLoache, Boston, MA (US); Zachary N. Russ, Boston, MA (US); Lauren Popov, Boston, MA (US); Ada Huang, Boston, MA (US); David Zong, Boston, MA (US); Jake Flood, Boston, MA (US); John Dueber, Boston, MA (US)

(21) Appl. No.: 19/114,043

(22) PCT Filed: Sep. 22, 2023
(86) PCT No.: PCT/US2023/074941

§ 371 Date: Mar. 21, 2025

Related U.S. Application Data
(60) Provisional application No. 63/409,592, filed on Sep. 23, 2022.

(51) Int. Cl. A61K 35/741; A61K 31/737; C12N 1/20; C12N 9/02; C12N 15/62; C12P 21/02; G01N 33/68

(52) U.S. Cl. CPC A61K 35/741; A61K 31/737; C12N 1/20; C12N 9/0069; C12N 15/62; C12P 21/02; G01N 33/6872; C07K 2319/036; C07K 2319/40; C07K 2319/50; C07K 2319/61; C12Y 113/12005

(57) ABSTRACT

Methods and compositions comprising recombinant fusion polypeptides that are engineered to allow for heterologous cargo secretion from a Bacteroides cell, into an extracellular environment are provided. Provided methods and compositions include recombinant fusion polypeptides comprising a secretion domain and a heterologous cargo domain. Methods and compositions for using said recombinant fusion polypeptides, e.g., for the treatment of a disease and/or disorder, are also provided.

### CROSS REFERENCE TO RELATED APPLICATIONS

This application claims the benefit of and priority to U.S. Provisional Patent Application No. 63/409,592, filed on Sep. 23, 2022, the entire contents of which are incorporated herein by reference.

### BACKGROUND

Systems that deliver polypeptide therapeutics to a subject are desirable for a variety of applications including treatment of infectious diseases, cancer, diabetes, and degenerative diseases, among others. A number of engineered bacterial cell systems have been developed for delivery of such polypeptide therapeutics, however, such systems often fail due to limited ability to colonize the gut of a human subject, low secretion efficiency, and/or toxicity of the therapeutic payload. Accordingly, there is a need for improved bacterial cell delivery systems that are not hindered by these limitations.

### SUMMARY

The genus Bacteroides consists of many bacteria that naturally populate the human gut in high abundance. Many species within this genus are considered commensal; accordingly, their presence in high abundance often correlates with a healthy microbiome. Consequently, the present disclosure appreciates that many Bacteroides strains offer promise as in situ living cell therapeutic delivery systems. However, there are currently limited strategies for secreting soluble, heterologous protein cargo from Bacteroides, especially at high titers, (e.g., titers higher than pg/mL values). One notable challenge for devising secretion systems for Gram-negative bacteria, such as Bacteroides, is due to secreted proteins needing to cross two (and in some cases, three, if including a eukaryotic cell membrane) phospholipid membranes in order to reach their final destination. See, e.g., Green and Mecsas (Microbiol Spectr. 2016 4(1):doi:10.1128/microbiolspec.VMBF-0012-2015). Despite these limitations in the field, the present disclosure surprisingly provides for, among other things, Bacteroides cell therapeutic delivery systems that allow for the delivery of various heterologous protein cargo, including, but not limited to, bioactive agents, therapeutic agents (e.g., cytokines, hormones, anti-inflammatories, antibodies, affibodies, nanobodies, enzymes, peptides, and derivatives or functional fragments thereof), and reporter polypeptides.

The present disclosure relates to the surprising discovery that certain bacterial polypeptides can be engineered to allow for heterologous cargo secretion from a Bacteroides cell (e.g., any species within the Bacteroides genus) into an extracellular environment. Without wishing to be bound by theory, it is thought that such engineered polypeptides allow for heterologous cargo secretion via a secretion domain. In many embodiments of the present disclosure, an engineered polypeptide is a recombinant fusion polypeptide comprising a secretion domain fused or linked to a heterologous cargo. Notably, recombinant fusion polypeptides of the present disclosure deliver heterologous cargo that is secreted from a cell and is free from outer-membrane vesicles (OMVs), e.g., soluble protein. Moreover, recombinant fusion polypeptides of the present disclosure have minimal toxicity to carrier Bacteroides cells, e.g., as judged by cell lysis. Further still, provided recombinant fusion polypeptides demonstrate wide capacity for types of heterologous cargo that can be delivered, with respect to size of protein as well as type (e.g., cytokine, hormone, anti-inflammatory, antibody, affibody, nanobody, enzyme, peptide, and so forth).

The present disclosure further provides compositions and methods useful in the treatment of diseases and disorders in a subject via administration of Bacteroides cells that express recombinant fusion polypeptides as described herein. It will be understood to a skilled artisan that such living cell secretion delivery systems of the present disclosure can be used to treat any disease or disorder where delivery of an agent (e.g., a bioactive agent, therapeutic agent, etc.) to the GI tract of a subject is beneficial.

The present disclosure provides for recombinant fusion polypeptides comprising: (i) a secretion domain comprising an amino acid sequence having at least 80% sequence identity to SEQ ID NO: 4; and (ii) a heterologous cargo domain. In some embodiments, a secretion domain comprises an amino acid sequence having at least 90% sequence identity to SEQ ID NO: 4. In some embodiments, a secretion domain comprises an amino acid sequence having at least 95% sequence identity to SEQ ID NO: 4. In some embodiments, a secretion domain comprises an amino acid sequence as set forth in SEQ ID NO: 4.

In some embodiments, a secretion domain comprises an amino acid sequence having at least 90% sequence identity to SEQ ID NO: 2. In some embodiments, a secretion domain comprises an amino acid sequence having at least 95% sequence identity to SEQ ID NO: 2. In some embodiments, a secretion domain comprises an amino acid sequence as set forth in SEQ ID NO: 2.

The present disclosure further provides recombinant fusion polypeptides comprising: (i) a secretion domain comprising an amino acid sequence having at least 80% sequence identity to SEQ ID NO: 67, and (ii) a heterologous cargo domain. In some embodiments, a secretion domain comprises an amino acid sequence having at least 90% sequence identity to SEQ ID NO: 67. In some embodiments, a secretion domain comprises an amino acid sequence having at least 95% sequence identity to SEQ ID NO: 67. In some embodiments, a secretion domain comprises an amino acid sequence as set forth in SEQ ID NO: 67.

In some embodiments, a heterologous cargo domain comprises an amino acid sequence of about 2 to about 20 amino acids, about 2 to about 10 amino acids, about 10 to about 50 amino acids, about 10 to about 40 amino acids, about 10 to about 30 amino acids, about 10 to about 20 amino acids, about 20 to about 50 amino acids, about 30 to about 50 amino acids, about 40 to about 50 amino acids, about 40 to about 60 amino acids, about 50 to about 500 amino acids, about 50 to about 450 amino acids, about 50 to about 400 amino acids, about 50 to about 350 amino acids, about 50 to about 300 amino acids, about 50 to about 250 amino acids, about 50 to about 200 amino acids, about 50 to about 100 amino acids, about 100 to about 500 amino acids, about 200 to about 500 amino acids, about 250 to about 500 amino acids, about 300 to about 500 amino acids, about 400 to about 500 amino acids, about 420 to about 500 amino acids, about 460 to about 500 amino acids, about 480 to about 500 amino acids, about 450 to about 600 amino acids, or about 500 to about 600 amino acids. In some embodiments, a heterologous domain comprises an amino acid sequence of about 30 to 40 amino acids. In some embodiments, a heterologous domain comprises an amino acid sequence of about 38 amino acids. In some embodiments, a heterologous domain comprises an amino acids sequence of about 450-500 amino acids. In some embodiments, a heterologous domain comprises an amino acid sequence of about 458 amino acids.

In some embodiments, a heterologous cargo domain comprises a reporter polypeptide. In some embodiments, a reporter polypeptide is selected from the group consisting of: a luciferase (e.g., GFP, RFP, or YFP), horse radish peroxidase (HRP), beta-glucosidase, and variants or combinations thereof. In some embodiments, a luciferase is a nanoluciferase.

In some embodiments, a recombinant fusion polypeptide of the present disclosure comprises an epitope tag. In some embodiments, an epitope tag is also a reporter polypeptide, e.g., as described herein. In some embodiments, an epitope tag is selected from the group consisting of: c-Myc, human influenza hemagglutinin (HA), FLAG, 3×FLAG, 6×His, glutathione-S-transferase (GST), maltose binding protein (MBP), GFP, RFP, mCherry, or variants or combinations thereof.

In some embodiments, a heterologous cargo domain comprises a bioactive moiety. In some embodiments, a heterologous cargo domain comprises a therapeutic polypeptide. In some embodiments, a therapeutic polypeptide is a cytokine, hormone, antibody, affibody, enzyme, bioactive peptide, or derivatives or functional fragments thereof.

In some embodiments, a heterologous cargo domain is fused directly or indirectly to the C-terminus of the secretion domain.

In some embodiments, a recombinant fusion polypeptide comprises a linker domain that links the secretion domain to the heterologous cargo domain. In some embodiments, a linker domain comprises a CL3 linker, a (GS)4 linker, a PQP linker, a NSQ linker, or a RAT linker. In some embodiments, a linker domain comprises a protease cleavage site. In some embodiments, a protease cleavage site is cleaved by one or more proteases that are present in the colon of a subject. In some embodiments, a protease cleavage site is cleaved by a protease selected from the group consisting of: chymotrypsin, chymotrypsin-like elastases, trypsin, tobacco etch virus (TEV), thrombin, human neutrophil elastase, cathepsin G, tryptase, chymase, proteinase 3, commensal enteric microbial proteases, and combinations thereof.

The present disclosure provides for polynucleotides encoding a recombinant fusion polypeptide as described herein.

In some embodiments, a polynucleotide as provided herein is operably linked to a promoter. In some embodiments, a promoter is an inducible promoter. In some embodiments, a promoter is a constitutive promoter.

The present disclosure also provides vectors comprising a polynucleotide as described herein. In some embodiments, a polynucleotide comprises a nucleotide sequence having at least 80% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 18, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 43, SEQ ID NO: 44, SEQ ID NO: 45, SEQ ID NO: 46, SEQ ID NO: 47, or SEQ ID NO: 66. In some embodiments, a polynucleotide comprises a nucleotide sequence having at least 90% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 18, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 43, SEQ ID NO: 44, SEQ ID NO: 45, SEQ ID NO: 46, SEQ ID NO: 47, or SEQ ID NO: 66. In some embodiments, a polynucleotide comprises a nucleotide sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 18, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 43, SEQ ID NO: 44, SEQ ID NO: 45, SEQ ID NO: 46, SEQ ID NO: 47, or SEQ ID NO: 66.

The present disclosure provides for Bacteroides cells comprising a polynucleotide as described herein, or a vector as described herein. In some embodiments, a Bacteroides cell is a Bacteroides vulgatus, Bacteroides thetaiotaomicron, Bacteroides koreensis, Bacteroides graminisolvens, Bacteroides uniformis, Bacteroides ovatus, Bacteroides xylanisolvens, Bacteroides stercoris, Bacteroides finegoldii, Bacteroides cellulosilyticus, Bacteroides kribbi, Bacteroides fragilis, Bacteroides massiliensis, Bacteroides dorei, Bacteroides faecis, Bacteroides salyersiae, or Bacteroides caccae cell.

In some embodiments, a Bacteroides cell comprises one or more transgenes that allow for utilization of a privileged nutrient as a carbon source or increase its ability to utilize a privileged nutrient as a carbon source. In some embodiments, a privileged nutrient is a marine polysaccharide. In some embodiments, a marine polysaccharide is porphyran or agarose.

The present disclosure provides for methods of treating a disease or disorder in a subject, the method comprising a step of administering to the subject a Bacteroides cell as described herein. In some embodiments, a disease or disorder is a gastrointestinal (GI) disease or disorder. In some embodiments, a GI disease or disorder is selected from the group consisting of: irritable bowel syndrome (IBS), cancer (e.g., colorectal cancer), infectious colitis, ulcerative colitis, Crohn's disease, ischemic colitis, radiation colitis, peptic ulcer disease, gastritis, gastroenteritis, and celiac disease. In some embodiments, a subject is further administered porphyran. In some embodiments, a porphyran is administered concurrently or sequentially with administration of the Bacteroides cell.

The present disclosure provides for methods of producing a recombinant fusion polypeptide from an engineered Bacteroides, the methods comprising culturing a Bacteroides cell comprising a polynucleotide as described herein, wherein the encoded recombinant fusion polypeptide is secreted outside of the Bacteroides cell. In some embodiments, a provided method further comprises collecting the recombinant fusion polypeptide.

The present disclosure provides for methods of quantifying protein secretion from an engineered Bacteroides, the methods comprising steps of: (i) culturing a Bacteroides cell capable of expressing a recombinant fusion polypeptide as described herein; and (ii) measuring the amount of recombinant fusion polypeptide that is secreted outside of the Bacteroides cell. In some embodiments, at least 70%, at least 80%, at least 90%, at least 95%, or at least 99% of the recombinant fusion polypeptide is secreted as soluble protein. In some embodiments, a secreted protein is not associated with outer membrane vesicles (OMVs).

The present disclosure also provides for methods of quantifying protein secretion from an engineered Bacteroides, the methods comprising steps of: (i) administering to a subject a Bacteroides cell capable of expressing a recombinant fusion polypeptide as provided herein; and (ii) measuring the amount of recombinant fusion polypeptide present in the feces of the subject, thereby to quantify the protein secretion from the engineered Bacteroides.

The present disclosure further provides for methods of assessing colonization by an engineered Bacteroides in the gut of a subject, the method comprising steps of: (i) administering to the subject a Bacteroides cell capable of expressing a recombinant fusion polypeptide as described herein; and (ii) measuring the amount of recombinant fusion polypeptide present in the feces of the subject, thereby to assess colonization by the engineered Bacteroides in the gut of the subject.

### BRIEF DESCRIPTION OF THE DRAWINGS

FIG. 1 shows a depiction of an exemplary living cell secretion system in which a genetically engineered Bacteroides cell is capable of expressing a recombinant fusion polypeptide, as provided herein. The depicted recombinant fusion protein includes an 04469 secretion domain and a nanoluciferase as heterologous protein cargo. Firefly luciferase is present in said cell as a cytosolic lysis control.

FIG. 2 shows a depiction of an exemplary recombinant fusion polypeptide provided by the instant disclosure. The depicted exemplary recombinant fusion polypeptide includes a signaling peptide domain (“SP’), a 4469 (also referred to as “04469”) secretion domain (e.g., as set forth in SEQ ID NO: 2), a cleavable linker domain (“CL3”), and a heterologous cargo domain (a nanoluciferase, or “NL”). Estimated weight in kDa is also shown for each component.

FIG. 3A shows a graph depicting luciferase signal in various samples derived from engineered Bacteroides vulgatus cells capable of expressing a 04469 secretion domain fused to nanoluciferase, e.g., as shown in FIG. 2. Total engineered Bacteroides cell culture luminescence is shown in blue (also labeled with “C” for culture). Bacteroides cell pellet luminescence and cell supernatant luminescence of said culture are shown in orange and gray, respectively (also labeled “P” for pellet and “S” for supernatant, respectively). As a reporter for amount of cell lysis, Bacteroides vulgatus cells were also made to express a cytoplasmic firefly luciferase, and total cell culture, cell supernatant, and cell pellet fractions were generated and luminescence measured. FIG. 3B shows an exemplary western blot analysis comparing nanoluciferase maintained in engineered Bacteroides cell pellet (P) versus cellular supernatant (S).

FIG. 4 shows a graph depicting secretion efficiency of nanoluciferase in various engineered Bacteroides strains capable of expressing a recombinant fusion polypeptide, as described herein. In this particular, a plasmid with 04469 secretion domain fused to a nanoluciferase was conjugated into Bacteroides strains of species throughout the genus. All conjugated strains exhibited efficient secretion as measured by the fraction of luminescence in media (the soluble supernatant fraction after centrifugation). Depicted results demonstrate, among other things, the extensive portability of recombinant fusion polypeptides provided in the present disclosure.

FIG. 5 shows a graph depicting luminescence signal (shown as % of max signal) from supernatants containing secreted nanoluciferase after being filtered with various molecular weight cutoff filters (no filter, 100 kDa, 50 kDa, 30 kDa, and 10 kDa). Cell supernatants were collected from engineered Bacteroides ovatus cells capable of expressing a recombinant fusion polypeptide, as described herein. In particular, a 04469 secretion domain was fused to nanoluciferase and conjugated into Bacteroides ovatus cells. Engineered Bacteroides cells produced a fusion protein expected to be approximately 40.8 kDa. A considerable drop in fusion protein (as measured by luciferase signal) was not observed until a molecular weight filter of 30 kDa was used, and almost all protein was filtered out of solution when a 10 kDa filter was used. These results indicate that secreted protein cargo behaves as a soluble protein that is not associated with the Bacteroides cells or larger complexes such as Outer Membrane Vesicles (OMVs).

FIG. 6 shows an exemplary western blot analysis of various heterologous cargo proteins secreted from engineered Bacteroides cells expressing a recombinant fusion polypeptide as described herein. In each case, heterologous cargo proteins were fused to the C-terminus of a 04469 secretion domain.

Protein cargos depicted include a solubility domain, a well-studied folded domain, small and large proteins of interest for therapeutic applications (e.g., nanobodies, affibody, peptide, cytokine). Epitope tags were additionally fused to each cargo, e.g., a 3×FLAG tag, for visualization by western blot. Predicted sizes of 04469-cargo fusions are listed under each pellet/supernatant lanes on the western blot for the respective strain tested. This predicted size is assuming the cleavage of the 3.2 kDa predicted signal peptide in the periplasm after transport through the inner membrane via the Sec translocon.

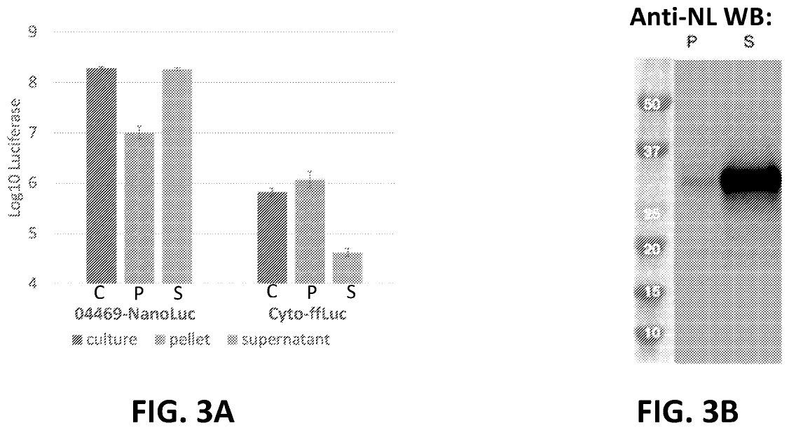

FIG. 7A shows an in vivo secretion study. Mice were gavaged with one of two engineered Bacteroides vulgatus strains capable of expressing either nanoluciferase with an N-terminal 04469 secretion domain fusion, or nanoluciferase alone that is expected to remain in the cytoplasm of tested Bacteroides cells. Both strains contained a porphyran utilization locus (PUL) that provides a unique environmental niche to support robust engraftment in the gastrointestinal tracts of the mice. Porphyran was supplemented in the chow for the entirety of the six day experiment. On the fifth day, quantitative PCR was performed to verify if tested mice were successfully engrafted. On the sixth day, mice were sacrificed and the cecum from each mouse was removed and centrifuged at a low speed to separate Bacteroides cells from mouse tissue. A supernatant fraction was then centrifuged at high speed to separate soluble, secreted material from insoluble material and bacterial cells. FIG. 7B shows a graph depicting colonization density of Bacteroides vulgatus strains in mice as described in FIG. 7A. Engraftment was measured by quantitative PCR in the feces of mice engrafted with both the experimental strains of nanoluciferase fused to 04469 and the control strain of nanoluciferase alone. Engraftment of both strains was verified. FIG. 7C shows a graph depicting luminescence of both of soluble and insoluble fractions taken from Bacteroides cells obtained in FIG. 7B, and processed per FIG. 7A. Similar to the in vitro studies shown in FIG. 3A, the luminescence was primarily in the supernatant fraction indicating efficient secretion

FIG. 8 shows a graph depicting luminescence signal (shown as RLUs) from cultured engineered Bacteroides strains. The total amount of RLUs are shown in the total bacterial culture, contained within the bacterial cell pellet (“Pellet”) or the extracellular environment (“Supernatant”), are depicted for each indicated strain (sZR1059, sZR1060, sZR1061, sZR1062, sZR1063, and sZR1064). Depicted Bacteroides strains express a diversity of recombinant polypeptide constructs in which variations on the native 04469 ribosomal binding site (RBS) such as “04469_P5B10” and “4469-PO17,” and N/C terminal linkers such as “RAT”, “(GS)4!”, “PQP!”, and “NSQ!” demonstrate a marked improvement in secretion of the heterologous nanoluciferase cargo relative to the base strain sZR1059.

FIG. 9 shows a graph depicting luminescence signal (shown as RLUs) from cultured engineered Bacteroides strains that are capable of expressing 04469 fused to nanoluciferase, where 04469 is either its full-length native sequence (“sWW3378”), or C-terminally truncated with the indicated number of terminal amino acid residues removed, e.g., “delta5” has 5 terminal amino acid residues removed, “delta8” has 8 terminal amino acid residues removed, and “delta12” has 12 terminal amino acid residues removed. The amount of secretion achieved by the various truncation constructs is indicated by comparing the amount of RLU signal in the culture supernatant (“sup”) to the culture bacterial cell pellet (“pel”). Comparison of depicted truncations constructs to full length 04469 shows that the terminal 5 amino acid residues are not necessary for efficient secretion of heterologous protein cargo into supernatant.

### DETAILED DESCRIPTION

### Definitions

Administration: As used herein, the term “administration” refers to administration of a provided engineered bacterial cell composition or method to a subject. Unless otherwise indicated, administration can refer to any route accepted as appropriate by the medical community for the disclosed living bacterial cell secretion systems. The present disclosure contemplates routes of administering that include oral (PO), intramuscular (IM), rectal (PR), intraperitoneal (IP), intragastric (IG), mucosal, enteral, and/or combinations of any of the foregoing. In some embodiments, administration is oral. In some embodiments, administration is intragastric. In some embodiments, administration is mucosal or enteral.

Agent: The term “agent” as used herein may refer to a compound or entity of any chemical class including, for example, polypeptides, nucleic acids, saccharides, lipids, small molecules, metals, or combinations thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent is biologically active, i.e., said agent is a bioactive agent. In some embodiments, a bioactive agent is or comprises a bioactive moiety that is responsible for bioactivity (e.g., physiological or pharmacological action). In some embodiments, an agent may be utilized in isolated or pure form; in some embodiments, an agent may be utilized in crude form. In some embodiments, potential agents are provided as collections or libraries, for example that may be screened to identify or characterize active agents (e.g., bioactive agents or therapeutic agents) within them. Some particular embodiments of agents that may be utilized in accordance with the present disclosure include small molecules, antibodies, antibody derivatives, antibody fragments, affibodies, nanobodies, aptamers, polypeptides, peptides, peptide mimetics, etc.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. Amino acids, including carboxy- and/or amino-terminal amino acids in peptides, can be modified by methylation, amidation, acetylation, and/or substitution with other chemical groups that can change the peptide's circulating half-life without adversely affecting their activity. Amino acids may participate in a disulfide bond. In some embodiments, the term “amino acid” is used to refer to a free amino acid; in some embodiments, the term “amino acid” refers to an amino acid residue of a polypeptide.

Animal: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In certain embodiments, an animal is suffering from and/or is susceptible to a disease or disorder. In some embodiments, an animal is suffering from a GI disease or disorder. In some embodiments, and animal is suffering from a disease or disorder that can be treated by administration of an agent (e.g., a bioactive agent, a therapeutic agent, etc.) that contacts the GI tract of said animal. In some embodiments, an animal may be a transgenic animal, genetically engineered animal, and/or a clone.

Antibody: As used herein, the term “antibody” refers to any immunoglobulin, whether natural or wholly or partially synthetically produced. All derivatives thereof which maintain specific binding ability are also included in the term. In some embodiments, the term “antibody” refers to any protein having a binding domain which is homologous or largely homologous to an immunoglobulin-binding domain. Antibody proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. In certain embodiments, an antibody may be a member of the IgG immunoglobulin class. As used herein, the term “antibody fragment” refers to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises at least about 200 amino acids. In some embodiments, an antibody may be a human antibody. In some embodiments, an antibody may be a humanized antibody.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any substance that has activity in a biological system (e.g., in a cell (in vivo or in vitro), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment, or a “bioactive moiety” or “bioactive domain.” In some embodiments, a bioactive portion or fragment, or bioactive moiety, facilitates one or more macromolecular interactions. In some embodiments, a bioactive portion or fragment, or bioactive moiety, facilitates protein-protein, protein-lipid, and/or protein-membrane interactions.

Expression cassette: As used herein, an “expression cassette” is a polynucleotide construct, generated recombinantly or synthetically, comprising regulatory sequences operably linked to a selected polynucleotide to facilitate expression of said selected polynucleotide in a host cell.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identical. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% similar.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM120 weight residue table, a length penalty of 12 and a gap penalty of 4. The percent identity between two nucleotide sequences can, alternatively, be determined using the GAP program in the GCG software package using an NWSgapdna.CMP matrix.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA.” “RNA.” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present disclosure. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be isolated or purified from natural sources, produced using recombinant expression systems and optionally isolated or purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present disclosure is directed to “unmodified nucleic acids,” meaning nucleic acids (e.g., polynucleotides and residues, including nucleotides and/or nucleosides) that have not been chemically modified in order to facilitate or achieve delivery.

Operably linked: As used herein the term “operably linked” refers to polynucleotide sequences or amino acid sequences placed into a functional relationship with one another. For instance, a promoter or enhancer is operably linked to a coding sequence if it regulates, or contributes to modulation of, the transcription of a coding sequence. Operably linked DNA sequences encoding regulatory sequences are typically contiguous to a coding sequence. However, enhancers can function when separated from a promoter by up to several kilobases or more. Additionally, multi-cistronic constructs can include multiple coding sequences which use only one promoter by including a 2A self-cleaving peptide, an IRES element, etc. Accordingly, some polynucleotide elements may be operably linked but not contiguous.

Patient: As used herein, the term “patient” or “subject” refers to any organism to which an provided compositions may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human.

Pharmaceutically acceptable: The term “pharmaceutically acceptable” as used herein, refers to substances that are suitable for use in contact with the tissues of human beings and animals without excessive toxicity, irritation, allergic response, or other problem or complication, commensurate with a reasonable benefit/risk ratio.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a biologically active portion thereof. Those of ordinary skill will appreciate that a protein can sometimes include more than one polypeptide chain, for example linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. Accordingly, in many cases, reference to a polypeptide also includes a peptide. In some embodiments, proteins are antibodies, antibody fragments, and/or biologically active portions thereof.

Rare carbohydrate: The term “rare carbohydrate” refers to a carbohydrate of interest that is utilized by less than 50% (e.g., less than 40%, less than 30%, less than 20%, less than 10%, less than 5%, less than 3%, less than 2%, less than 1%, less than 0.5%, less than 0.2%, less than 0.1%, or none; e.g., less than 30%, less than 10%, less than 3%, less than 1%, less than 0.3%, less than 0.1%, less than 0.03%, less than 0.01%, less than 0.003%, less than 0.001%, less than 0.0001%, or none) of other bacterial cells present in a target organ such as the gut (i.e., cells ‘other’ than the introduced/genetically modified bacteria, e.g., cells of the resident population prior to introduction). Thus, in some cases (e.g., in any of the methods of the disclosure), a rare carbohydrate of interest is one that can be utilized by less than 50% (e.g., less than 40%, less than 30%, less than 20%, less than 10%, less than 5%, less than 3%, less than 2%, less than 1%, less than 0.5%, less than 0.2%, less than 0.1%, or none; e.g., less than 30%, less than 10%, less than 3%, less than 1%, less than 0.3%, less than 0.1%, less than 0.03%, less than 0.01%, less than 0.003%, less than 0.001%, less than 0.0001%, or none) of other bacterial cells present in the organ. In some cases (e.g., in any of the methods of the disclosure), the rare carbohydrate of interest is one that can be utilized by less than 20% (e.g., less than 10%, less than 5%, less than 3%, less than 2%, less than 1%, less than 0.5%, less than 0.2%, less than 0.1%, or none) of other bacterial cells present in the organ. In some cases (e.g., in any of the methods of the disclosure), the rare carbohydrate of interest is one that can be utilized by less than 5% (e.g., less than 3%, less than 2%, less than 1%, less than 0.5%, less than 0.2%, less than 0.1%, or none) of other bacterial cells present in the organ. In some cases (e.g., in any of the methods of the disclosure), the rare carbohydrate of interest is one that can be utilized by less than 2% (e.g., less than 1%, less than 0.5%, less than 0.2%, less than 0.1%, or none) of other bacterial cells present in the organ. In some cases (e.g., in any of the methods of the disclosure), the rare carbohydrate of interest is one that can be utilized by less than 0.5% (e.g., less than 0.2%, less than 0.1%, or none) of other bacterial cells present in the organ. In some cases (e.g., in any of the methods of the disclosure), the rare carbohydrate of interest is one that can be utilized by none of the other bacterial cells present in the organ.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic agent: As used herein, the phrase “therapeutic agent” in general refers to any agent that elicits a desired pharmacological/therapeutic effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, the term “therapeutically effective amount” or “therapeutically effective dose” means an amount that is sufficient, when administered to a population suffering from or susceptible to a disease, disorder, and/or condition in accordance with a therapeutic dosing regimen, to treat the disease, disorder, and/or condition (e.g., a GI disease or disorder). In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. In some embodiments, reference to a therapeutically effective amount may be a reference to an amount as measured in one or more specific tissues. Those of ordinary skill in the art will appreciate that, in some embodiments, a therapeutically effective amount may be formulated and/or administered in a single dose. In some embodiments, a therapeutically effective agent may be formulated and/or administered in a plurality of doses, for example, as part of a dosing regimen. In some embodiments, a unit dose that contains a dose to be administered as part of a therapeutically effective regimen is referred to as containing a “therapeutically effective” amount even though administration of only that single unit dose might not be expected to correlate with therapeutic effectiveness (e.g., across a population).

Therapeutic regimen: A “therapeutic regimen”, as that term is used herein, refers to a dosing regimen whose administration across a relevant population is correlated with a desired or beneficial therapeutic outcome

Treatment: As used herein, the term “treatment” (also “treat” or “treating”) refers to any administration of a substance (e.g., a provided composition) that partially or completely alleviates, ameliorates, relives, inhibits, delays onset of, reduces severity of, and/or reduces incidence of one or more symptoms, features, and/or causes of a particular disease, disorder, and/or condition (e.g., a GI disease or disorder). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Vector: As used herein, “vector” refers to a nucleic acid molecule capable of transporting another nucleic acid to which it is associated. The terms “vector” and “plasmid” may be used interchangeably. In some embodiment, vectors are capable of extra-chromosomal replication and/or expression of nucleic acids to which they are linked in a host cell such as a eukaryotic and/or prokaryotic cell. Vectors capable of directing the expression of operatively linked genes are referred to herein as “expression vectors.” An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes.

### Engineered Polypeptides

The present disclosure provides for, among other things, engineered polypeptides, e.g., recombinant fusion polypeptides, that allow for the secretion of soluble, heterologous protein cargo across a membrane of a Bacteroides cell. Remarkably, recombinant fusion polypeptides comprising soluble heterologous cargo can be secreted from a Bacteroides cell with high efficiency and are free from outer-membrane vesicles (OMVs). Moreover, recombinant fusion polypeptides of the present disclosure have minimal toxicity to carrier Bacteroides cells, e.g., as judged by cell lysis. Further still, recombinant fusion polypeptides can comprise a wide variety of heterologous cargo for secretion, with respect to size of protein as well as type (e.g., cytokine, hormone, anti-inflammatory, antibody, affibody, nanobody, enzyme, peptide, and so forth).

Engineered polypeptides of the present disclosure may be fusion proteins, e.g., recombinant fusion polypeptides comprising a secretion domain fused, or linked, to a heterologous polypeptide region, or heterologous cargo domain. Additional moieties may be included in engineered polypeptides provided herein, including, but not limited to, epitope tags, linkers, and reporter polypeptides.

In some embodiments, recombinant fusion polypeptides of the present disclosure comprise a secretion domain, e.g., a 04469 polypeptide as described herein, and a heterologous cargo domain. Any heterologous protein cargo may be used in disclosed engineered polypeptides to the extent that secretion of the cargo from a cell is achieved. In some embodiments, a recombinant fusion polypeptide comprises a signaling peptide domain, e.g., as provided herein. A signaling peptide domain can be a part of a secretion domain that is cleaved off during secretion, e.g., in the periplasm. In some embodiments, a signaling peptide domain is or comprises a bacterial signaling peptide domain. In certain embodiments, the signaling peptide domain comprises amino acid sequence MKKMRFLLVLLVGISIVSCT (amino acids 1-20 of SEQ ID NO: 2.) In some embodiments, the signaling peptide domain comprises amino acid sequence MLNLNYVKMKKMRFLLVLLVGISIVSCT (amino acids 1-28 of SEQ ID NO: 67). In some embodiments, a signaling peptide domain is cleaved between two neighboring amino acid residues within amino acid sequence SIVSCTNDLDE (amino acids 15-25 of SEQ ID NO: 2). In some embodiments, a recombinant fusion polypeptide comprises an epitope tag. An epitope tag used in accordance with the present disclosure may comprise or consist of any epitope commonly used in the field, including those described herein. In some embodiments, a heterologous cargo domain is fused directly to a secretion domain. In some embodiments, a heterologous cargo domain is fused indirectly to a secretion domain. In some embodiments, a heterologous cargo domain is fused directly to the C-terminus of a secretion domain. In some embodiments, a heterologous cargo domain is fused indirectly to the C-terminus of a secretion domain, e.g., via a linker. In some embodiments, a recombinant fusion polypeptide comprises one or more linker domains (e.g., a first linker, a second linker, a third linker, a fourth linker, and so forth). It will be understood that a linker domain used in accordance with the present disclosure may comprise or consist of any amino acid linker sequence commonly used by skilled artisans in the field, including, but not limited to, those described herein. In some embodiments, two or more linkers domains can have the same sequence or a different sequence. In some embodiments, a secretion domain and a heterologous cargo domain are fused, or linked, by a linker domain. In some embodiments, a heterologous cargo domain and an epitope tag are fused, or linked, by a linker domain. In some embodiments, a heterologous cargo domain comprises a biologically active portion or fragment (e.g., a bioactive moiety, a therapeutic moiety, etc.). In some embodiments, a heterologous cargo domain is a biologically active portion or fragment (e.g., a bioactive moiety, therapeutic moiety, etc.).

In some embodiments, a recombinant fusion polypeptide as provided herein comprises, from N-terminus to C-terminus, a secretion domain and a heterologous cargo domain. In some embodiments, a recombinant fusion polypeptide as provided herein comprises, from N-terminus to C-terminus, a secretion domain, a linker domain, and a heterologous cargo domain.

In some embodiments, a recombinant fusion polypeptide comprises, from N-terminus to C-terminus, a signal peptide domain, a secretion domain, and a heterologous cargo domain. In some embodiments, a recombinant fusion polypeptide comprises, from N-terminus to C-terminus, a signal peptide domain, a secretion domain, a linker domain, and a heterologous cargo domain. In some embodiments, a recombinant fusion polypeptide comprises, from N-terminus to C-terminus, a signal peptide domain, a linker domain, a secretion domain, and a heterologous cargo domain. In some embodiments, a recombinant fusion polypeptide comprises, from N-terminus to C-terminus, a signal peptide domain, a first linker domain, a secretion domain, a second linker domain, and a heterologous cargo domain.

In some embodiments, a recombinant fusion polypeptide comprises, from N-terminus to C-terminus, a secretion domain, a heterologous cargo domain, and an epitope tag. In some embodiments, a recombinant fusion polypeptide comprises, from N-terminus to C-terminus, a secretion domain, a linker domain, a heterologous cargo domain, and an epitope tag. In some embodiments, a recombinant fusion polypeptide comprises, from N-terminus to C-terminus, a secretion domain, a heterologous cargo domain, a linker domain, and an epitope tag. In some embodiments, a recombinant fusion polypeptide comprises, from N-terminus to C-terminus, a secretion domain, a first linker domain, a heterologous cargo domain, a second linker domain, and an epitope tag.

In some embodiments, a recombinant fusion polypeptide comprises, from N-terminus to C-terminus, a signal peptide domain, a secretion domain, a heterologous cargo domain, and an epitope tag. In some embodiments, a recombinant fusion polypeptide comprises, from N-terminus to C-terminus, a signal peptide domain, a secretion domain, a linker domain, a heterologous cargo domain, and an epitope tag. In some embodiments, a recombinant fusion polypeptide comprises, from N-terminus to C-terminus, a signal peptide domain, a secretion domain, a heterologous cargo domain, a linker domain, and an epitope tag. In some embodiments, a recombinant fusion polypeptide comprises, from N-terminus to C-terminus, a signal peptide domain, a linker domain, a secretion domain, a heterologous cargo domain, and an epitope tag. In some embodiments, a recombinant fusion polypeptide comprises, from N-terminus to C-terminus, a signal peptide domain, a secretion domain, a first linker domain, a heterologous cargo domain, a second linker domain, and an epitope tag. In some embodiments, a recombinant fusion polypeptide comprises, from N-terminus to C-terminus, a signal peptide domain, a first linker domain, a secretion domain, a second linker domain, a heterologous cargo domain, and an epitope tag. In some embodiments, a recombinant fusion polypeptide comprises, from N-terminus to C-terminus, a signal peptide domain, a first linker domain, a secretion domain, a heterologous cargo domain, a second linker domain, and an epitope tag. In some embodiments, a recombinant fusion polypeptide comprises, from N-terminus to C-terminus, a signal peptide domain, a first linker domain, a secretion domain, a second linker domain, a heterologous cargo domain, a third linker domain, and an epitope tag.

In some embodiments of the present disclosure, a recombinant fusion polypeptide comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having at least about 80% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having at least about 90% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having at least about 95% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having at least about 96% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having at least about 97% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having at least about 98% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having at least about 99% sequence identity to any one of SEQ ID NOs: 6, 8, 10, 12, 14, 16, 19, 23, 25, 27, 29, 31, 33, 35, 37, 39, or 41.

In some embodiments of the present disclosure, a recombinant fusion polypeptide comprises an amino acid sequence having about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having about 80% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having about 90% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having about 95% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having about 96% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having about 97% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having about 98% sequence identity to any one of SEQ ID NOs: 6, 8, 10, 12, 14, 16, 19, 23, 25, 27, 29, 31, 33, 35, 37, 39, or 41. In some embodiments, a recombinant fusion polypeptide comprises an amino acid sequence having about 99% sequence identity to any one of SEQ ID NOs: 6, 8, 10, 12, 14, 16, 19, 23, 25, 27, 29, 31, 33, 35, 37, 39, or 41.

In some embodiments, a recombinant fusion polypeptide comprises an amino acid sequence as set forth in any one of SEQ ID NOs: 6, 8, 10, 12, 14, 16, 19, 23, 25, 27, 29, 31, 33, 35, 37, 39, or 41. In some embodiments, a recombinant fusion polypeptide consists essentially of an amino acid sequence as set forth in any one of SEQ ID NOs: 6, 8, 10, 12, 14, 16, 19, 23, 25, 27, 29, 31, 33, 35, 37, 39, or 41. In some embodiments, a recombinant fusion polypeptide consists of an amino acid sequence as set forth in any one of SEQ ID NOs: 6, 8, 10, 12, 14, 16, 19, 23, 25, 27, 29, 31, 33, 35, 37, 39, or 41.

In some embodiments of the present disclosure, a recombinant fusion polypeptide is encoded in a polynucleotide comprising a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having at least about 80% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having at least about 90% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having at least about 95% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having at least about 96% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having at least about 97% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having at least about 98% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having at least about 99% sequence identity to any one of SEQ ID NOs: 5, 7, 9, 11, 13, 15, 18, 22, 24, 26, 28, 30, 32, 34, 36, 38, or 40.

In some embodiments of the present disclosure, a recombinant fusion polypeptide is encoded in a polynucleotide comprising a nucleotide sequence having about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having about 80% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having about 90% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having about 95% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having about 96% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having about 97% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having about 98% sequence identity to any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide comprising a nucleotide sequence having about 99% sequence identity to any one of SEQ ID NOs: 5, 7, 9, 11, 13, 15, 18, 22, 24, 26, 28, 30, 32, 34, 36, 38, or 40.

In some embodiments, a recombinant fusion polypeptide is encoded in a polynucleotide comprising a nucleotide sequence as set forth in any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide consisting essentially of a nucleotide sequence as set forth in any one of SEQ ID NOs: 5, 7, 9, 11, 13, 15, 18, 22, 24, 26, 28, 30, 32, 34, 36, 38, or 40. In some embodiments, a recombinant fusion polypeptide is encoded in a polynucleotide consisting of a nucleotide sequence as set forth in any one of SEQ ID NOs: 5, 7, 9, 11, 13, 15, 18, 22, 24, 26, 28, 30, 32, 34, 36, 38, or 40.

In some embodiments, engineered polypeptides of the present disclosure (e.g., recombinant fusion polypeptides) comprise a solubility domain that increases secretion. In some embodiments a solubility domain comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 49. In some embodiments a solubility domain comprises an amino acid sequence having about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% sequence identity to SEQ ID NO: 49.

In some embodiments, a solubility domain is encoded in a polynucleotide comprising a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 48. In some embodiments, a solubility domain is encoded in a polynucleotide comprising a nucleotide sequence having about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% sequence identity to SEQ ID NO: 48.

In some embodiments, a recombinant fusion polypeptide comprises an amino acid sequence of at least about 100 amino acids, at least about 125 amino acids, at least about 150 amino acids, at least 175 amino acids, at least 200 amino acids at least about 500 amino acids, at least about 550 amino acids, at least about 600 amino acids, at least about 650 amino acids, at least about 700 amino acids, at least about 750 amino acids, at least about 800 amino acids, at least about 850 amino acids, at least about 900 amino acids, at least about 950 amino acids, at least about 1000 amino acids, at least about 1100 amino acids, at least about 1200 amino acids, at least about 1300 amino acids, at least about 1400 amino acids, at least about 1500 amino acids, at least about 1600 amino acids, at least about 1700 amino acids, at least about 1800 amino acids, at least about 1900 amino acids, or at least about 2000 amino acids in length.

In some embodiments, a recombinant fusion polypeptide comprises an amino acid sequence of about 100 to about 800 amino acids, about 100 to about 700 amino acids, about 100 to about 600 amino acids, about 100 to about 500 amino acids, about 100 to about 400 amino acids, about 100 to about 300 amino acids, about 100 to about 250 amino acids, about 100 to about 200 amino acids, about 100 to about 180 amino acids, about 100 to about 160 amino acids, about 100 to about 140 amino acids, about 100 to about 120 amino acids, about 500 to about 2000 amino acids, about 600 to about 2000 amino acids, about 700 to about 200 amino acids, about 800 to about 2000 amino acid, about 900 to about 2000 amino acids, about 1000 to about 2000 amino acids, about 1250 to about 2000 amino acids, about 1500 to about 2000 amino acids, about 1750 to about 2000 amino acids, about 1900 to about 2000 amino acids, about 500 to about 1500 amino acids, about 500 to about 1000 amino acids, about 500 to about 900 amino acids, about 500 to about 800 amino acids, about 500 to about 700 amino acids, or about 500 to about 600 amino acids in length.

### Secretion Domains

The disclosure relates in part to secretion domains that, when fused to a cargo protein, allow for heterologous cargo secretion from a Bacteroides cell. An exemplary secretion domain is set forth in SEQ ID NO: 2. SEQ ID NO: 2 comprises a signaling peptide domain that is cleaved off during processing in the periplasm. Accordingly, in some embodiments of the present disclosure, a secretion domain comprises a polypeptide with an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 2. In some embodiments, a secretion domain comprises a polypeptide with an amino acid sequence having at least about 80% sequence identity to SEQ ID NO: 2. In some embodiments, a secretion domain comprises a polypeptide with an amino acid sequence having at least about 90% sequence identity to SEQ ID NO: 2. In some embodiments, a secretion domain comprises a polypeptide with an amino acid sequence having at least about 95% sequence identity to SEQ ID NO: 2. In some embodiments, a secretion domain comprises a polypeptide with an amino acid sequence having at least about 96% sequence identity to SEQ ID NO: 2. In some embodiments, a secretion domain comprises a polypeptide with an amino acid sequence having at least about 97% sequence identity to SEQ ID NO: 2. In some embodiments, a secretion domain comprises a polypeptide with an amino acid sequence having at least about 98% sequence identity to SEQ ID NO: 2. In some embodiments, a secretion domain comprises a polypeptide with an amino acid sequence having at least about 99% sequence identity to SEQ ID NO: 2.

In some embodiments, a secretion domain comprises a polypeptide with an amino acid sequence having about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% sequence identity to SEQ ID NO: 2. In some embodiments, a secretion domain comprises a polypeptide with an amino acid sequence having about 80% sequence identity to SEQ ID NO: 2. In some embodiments, a secretion domain comprises a polypeptide with an amino acid sequence having about 90% sequence identity to SEQ ID NO: 2. In some embodiments, a secretion domain comprises a polypeptide with an amino acid sequence having about 95% sequence identity to SEQ ID NO: 2. In some embodiments, a secretion domain comprises a polypeptide with an amino acid sequence having about 96% sequence identity to SEQ ID NO: 2. In some embodiments, a secretion domain comprises a polypeptide with an amino acid sequence having about 97% sequence identity to SEQ ID NO: 2. In some embodiments, a secretion domain comprises a polypeptide with an amino acid sequence having about 98% sequence identity to SEQ ID NO: 2. In some embodiments, a secretion domain comprises a polypeptide with an amino acid sequence having about 99% sequence identity to SEQ ID NO: 2. In some embodiments, a secretion domain comprises a polypeptide with an amino acid sequence having about 100% sequence identity to SEQ ID NO: 2.

In some embodiments, a secretion domain comprises a polypeptide with an amino acid sequence as set forth in SEQ ID NO: 2. In some embodiments, a secretion domain consists essentially of a polypeptide with an amino acid sequence as set forth in SEQ ID NO: 2. In some embodiments, a secretion domain consists of a polypeptide with an amino acid sequence as set forth in SEQ ID NO: 2.

Another exemplary secretion domain is set forth in SEQ ID NO: 4. SEQ ID NO: 4 comprises a 04469 secretion domain. In some embodiments of the present disclosure, a secretion domain comprises a polypeptide with an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 4. In some embodiments, a secretion domain comprises a polypeptide with an amino acid sequence having at least about 80% sequence identity to SEQ ID NO: 4. In some embodiments, a secretion domain comprises a polypeptide with an amino acid sequence having at least about 90% sequence identity to SEQ ID NO: 4. In some embodiments, a secretion domain comprises a polypeptide with an amino acid sequence having at least about 95% sequence identity to SEQ ID NO: 4. In some embodiments, a secretion domain comprises a polypeptide with an amino acid sequence having at least about 96% sequence identity to SEQ ID NO: 4. In some embodiments, a secretion domain comprises a polypeptide with an amino acid sequence having at least about 97% sequence identity to SEQ ID NO: 4. In some embodiments, a secretion domain comprises a polypeptide with an amino acid sequence having at least about 98% sequence identity to SEQ ID NO: 4. In some embodiments, a secretion domain comprises a polypeptide with an amino acid sequence having at least about 99% sequence identity to SEQ ID NO: 4.

In some embodiments, a secretion domain comprises a polypeptide with an amino acid sequence having about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% sequence identity to SEQ ID NO: 4. In some embodiments, a secretion domain comprises a polypeptide with an amino acid sequence having about 80% sequence identity to SEQ ID NO: 4. In some embodiments, a secretion domain comprises a polypeptide with an amino acid sequence having about 90% sequence identity to SEQ ID NO: 4. In some embodiments, a secretion domain comprises a polypeptide with an amino acid sequence having about 95% sequence identity to SEQ ID NO: 4. In some embodiments, a secretion domain comprises a polypeptide with an amino acid sequence having about 96% sequence identity to SEQ ID NO: 4. In some embodiments, a secretion domain comprises a polypeptide with an amino acid sequence having about 97% sequence identity to SEQ ID NO: 4. In some embodiments, a secretion domain comprises a polypeptide with an amino acid sequence having about 98% sequence identity to SEQ ID NO: 4. In some embodiments, a secretion domain comprises a polypeptide with an amino acid sequence having about 99% sequence identity to SEQ ID NO: 4. In some embodiments, a secretion domain comprises a polypeptide with an amino acid sequence having about 100% sequence identity to SEQ ID NO: 4.

In some embodiments, a secretion domain comprises a polypeptide with an amino acid sequence as set forth in SEQ ID NO: 4. In some embodiments, a secretion domain consists essentially of a polypeptide with an amino acid sequence as set forth in SEQ ID NO: 4. In some embodiments, a secretion domain consists of a polypeptide with an amino acid sequence as set forth in SEQ ID NO: 4.

Another exemplary secretion domain is set forth in SEQ ID NO: 67. SEQ ID NO: 67 comprises a signaling peptide domain that is cleaved of during processing in the periplasm. The 04469 secretion domain of SEQ ID NO: 67 comprises an additional eight (8) amino acids at the N-terminal portion of the signaling peptide domain as compared to SEQ ID NO: 2. In some embodiments of the present disclosure, a secretion domain comprises a polypeptide with an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 67. In some embodiments, a secretion domain comprises a polypeptide with an amino acid sequence having at least about 80% sequence identity to SEQ ID NO: 67. In some embodiments, a secretion domain comprises a polypeptide with an amino acid sequence having at least about 90% sequence identity to SEQ ID NO: 67. In some embodiments, a secretion domain comprises a polypeptide with an amino acid sequence having at least about 95% sequence identity to SEQ ID NO: 67. In some embodiments, a secretion domain comprises a polypeptide with an amino acid sequence having at least about 96% sequence identity to SEQ ID NO: 67. In some embodiments, a secretion domain comprises a polypeptide with an amino acid sequence having at least about 97% sequence identity to SEQ ID NO: 67. In some embodiments, a secretion domain comprises a polypeptide with an amino acid sequence having at least about 98% sequence identity to SEQ ID NO: 67. In some embodiments, a secretion domain comprises a polypeptide with an amino acid sequence having at least about 99% sequence identity to SEQ ID NO: 67.

In some embodiments, a secretion domain comprises a polypeptide with an amino acid sequence having about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% sequence identity to SEQ ID NO: 67. In some embodiments, a secretion domain comprises a polypeptide with an amino acid sequence having about 80% sequence identity to SEQ ID NO: 67. In some embodiments, a secretion domain comprises a polypeptide with an amino acid sequence having about 90% sequence identity to SEQ ID NO: 67. In some embodiments, a secretion domain comprises a polypeptide with an amino acid sequence having about 95% sequence identity to SEQ ID NO: 67. In some embodiments, a secretion domain comprises a polypeptide with an amino acid sequence having about 96% sequence identity to SEQ ID NO: 67. In some embodiments, a secretion domain comprises a polypeptide with an amino acid sequence having about 97% sequence identity to SEQ ID NO: 67. In some embodiments, a secretion domain comprises a polypeptide with an amino acid sequence having about 98% sequence identity to SEQ ID NO: 67. In some embodiments, a secretion domain comprises a polypeptide with an amino acid sequence having about 99% sequence identity to SEQ ID NO: 67. In some embodiments, a secretion domain comprises a polypeptide with an amino acid sequence having about 100% sequence identity to SEQ ID NO: 67.

In some embodiments, a secretion domain comprises a polypeptide with an amino acid sequence as set forth in SEQ ID NO: 67. In some embodiments, a secretion domain consists essentially of a polypeptide with an amino acid sequence as set forth in SEQ ID NO: 67. In some embodiments, a secretion domain consists of a polypeptide with an amino acid sequence as set forth in SEQ ID NO: 67.

In some embodiments of the present disclosure, a secretion domain is encoded in a polynucleotide comprising a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 1. In some embodiments, a secretion domain is encoded in a polynucleotide comprising a nucleotide sequence having at least about 80% sequence identity to SEQ ID NO: 1. In some embodiments, a secretion domain is encoded in a polynucleotide comprising a nucleotide sequence having at least about 90% sequence identity to SEQ ID NO: 1. In some embodiments, a secretion domain is encoded in a polynucleotide comprising a nucleotide sequence having at least about 95% sequence identity to SEQ ID NO: 1. In some embodiments, a secretion domain is encoded in a polynucleotide comprising a nucleotide sequence having at least about 96% sequence identity to SEQ ID NO: 1. In some embodiments, a secretion domain is encoded in a polynucleotide comprising a nucleotide sequence having at least about 97% sequence identity to SEQ ID NO: 1. In some embodiments, a secretion domain is encoded in a polynucleotide comprising a nucleotide sequence having at least about 98% sequence identity to SEQ ID NO: 1. In some embodiments, a secretion domain is encoded in a polynucleotide comprising a nucleotide sequence having at least about 99% sequence identity to SEQ ID NO: 1.

In some embodiments of the present disclosure, a secretion domain is encoded in a polynucleotide comprising a nucleotide sequence having about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% sequence identity to SEQ ID NO: 1. In some embodiments, a secretion domain is encoded in a polynucleotide comprising a nucleotide sequence having about 80% sequence identity to SEQ ID NO: 1. In some embodiments, a secretion domain is encoded in a polynucleotide comprising a nucleotide sequence having about 90% sequence identity to SEQ ID NO: 1. In some embodiments, a secretion domain is encoded in a polynucleotide comprising a nucleotide sequence having about 95% sequence identity to SEQ ID NO: 1. In some embodiments, a secretion domain is encoded in a polynucleotide comprising a nucleotide sequence having about 96% sequence identity to SEQ ID NO: 1. In some embodiments, a secretion domain is encoded in a polynucleotide comprising a nucleotide sequence having about 97% sequence identity to SEQ ID NO: 1. In some embodiments, a secretion domain is encoded in a polynucleotide comprising a nucleotide sequence having about 98% sequence identity to SEQ ID NO: 1. In some embodiments, a secretion domain is encoded in a polynucleotide comprising a nucleotide sequence having about 99% sequence identity to SEQ ID NO: 1. In some embodiments, a secretion domain is encoded in a polynucleotide comprising a nucleotide sequence having about 100% sequence identity to SEQ ID NO: 1.

In some embodiments, a secretion domain is encoded in a polynucleotide comprising a nucleotide sequence as set forth in SEQ ID NO: 1. In some embodiments, a secretion domain is encoded in a polynucleotide consisting essentially of a nucleotide sequence as set forth in SEQ ID NO: 1. In some embodiments, a secretion domain is encoded in a polynucleotide consisting of a nucleotide sequence as set forth in SEQ ID NO: 1.

In some embodiments of the present disclosure, a secretion domain is encoded in a polynucleotide comprising a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 3. In some embodiments, a secretion domain is encoded in a polynucleotide comprising a nucleotide sequence having at least about 80% sequence identity to SEQ ID NO: 3. In some embodiments, a secretion domain is encoded in a polynucleotide comprising a nucleotide sequence having at least about 90% sequence identity to SEQ ID NO: 3. In some embodiments, a secretion domain is encoded in a polynucleotide comprising a nucleotide sequence having at least about 95% sequence identity to SEQ ID NO: 3. In some embodiments, a secretion domain is encoded in a polynucleotide comprising a nucleotide sequence having at least about 96% sequence identity to SEQ ID NO: 3. In some embodiments, a secretion domain is encoded in a polynucleotide comprising a nucleotide sequence having at least about 97% sequence identity to SEQ ID NO: 3. In some embodiments, a secretion domain is encoded in a polynucleotide comprising a nucleotide sequence having at least about 98% sequence identity to SEQ ID NO: 3. In some embodiments, a secretion domain is encoded in a polynucleotide comprising a nucleotide sequence having at least about 99% sequence identity to SEQ ID NO: 3.

In some embodiments of the present disclosure, a secretion domain is encoded in a polynucleotide comprising a nucleotide sequence having about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% sequence identity to SEQ ID NO: 3. In some embodiments, a secretion domain is encoded in a polynucleotide comprising a nucleotide sequence having about 80% sequence identity to SEQ ID NO: 3. In some embodiments, a secretion domain is encoded in a polynucleotide comprising a nucleotide sequence having about 90% sequence identity to SEQ ID NO: 3. In some embodiments, a secretion domain is encoded in a polynucleotide comprising a nucleotide sequence having about 95% sequence identity to SEQ ID NO: 3. In some embodiments, a secretion domain is encoded in a polynucleotide comprising a nucleotide sequence having about 96% sequence identity to SEQ ID NO: 3. In some embodiments, a secretion domain is encoded in a polynucleotide comprising a nucleotide sequence having about 97% sequence identity to SEQ ID NO: 3. In some embodiments, a secretion domain is encoded in a polynucleotide comprising a nucleotide sequence having about 98% sequence identity to SEQ ID NO: 3. In some embodiments, a secretion domain is encoded in a polynucleotide comprising a nucleotide sequence having about 99% sequence identity to SEQ ID NO: 3. In some embodiments, a secretion domain is encoded in a polynucleotide comprising a nucleotide sequence having about 100% sequence identity to SEQ ID NO: 3.

In some embodiments, a secretion domain is encoded in a polynucleotide comprising a nucleotide sequence as set forth in SEQ ID NO: 3. In some embodiments, a secretion domain is encoded in a polynucleotide consisting essentially of a nucleotide sequence as set forth in SEQ ID NO: 3. In some embodiments, a secretion domain is encoded in a polynucleotide consisting of a nucleotide sequence as set forth in SEQ ID NO: 3.

In some embodiments of the present disclosure, a secretion domain is encoded in a polynucleotide comprising a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 66. In some embodiments, a secretion domain is encoded in a polynucleotide comprising a nucleotide sequence having at least about 80% sequence identity to SEQ ID NO: 66. In some embodiments, a secretion domain is encoded in a polynucleotide comprising a nucleotide sequence having at least about 90% sequence identity to SEQ ID NO: 66. In some embodiments, a secretion domain is encoded in a polynucleotide comprising a nucleotide sequence having at least about 95% sequence identity to SEQ ID NO: 66. In some embodiments, a secretion domain is encoded in a polynucleotide comprising a nucleotide sequence having at least about 96% sequence identity to SEQ ID NO: 66. In some embodiments, a secretion domain is encoded in a polynucleotide comprising a nucleotide sequence having at least about 97% sequence identity to SEQ ID NO: 66. In some embodiments, a secretion domain is encoded in a polynucleotide comprising a nucleotide sequence having at least about 98% sequence identity to SEQ ID NO: 66. In some embodiments, a secretion domain is encoded in a polynucleotide comprising a nucleotide sequence having at least about 99% sequence identity to SEQ ID NO: 66.

In some embodiments of the present disclosure, a secretion domain is encoded in a polynucleotide comprising a nucleotide sequence having about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% sequence identity to SEQ ID NO: 66. In some embodiments, a secretion domain is encoded in a polynucleotide comprising a nucleotide sequence having about 80% sequence identity to SEQ ID NO: 66. In some embodiments, a secretion domain is encoded in a polynucleotide comprising a nucleotide sequence having about 90% sequence identity to SEQ ID NO: 66. In some embodiments, a secretion domain is encoded in a polynucleotide comprising a nucleotide sequence having about 95% sequence identity to SEQ ID NO: 66. In some embodiments, a secretion domain is encoded in a polynucleotide comprising a nucleotide sequence having about 96% sequence identity to SEQ ID NO: 66. In some embodiments, a secretion domain is encoded in a polynucleotide comprising a nucleotide sequence having about 97% sequence identity to SEQ ID NO: 66. In some embodiments, a secretion domain is encoded in a polynucleotide comprising a nucleotide sequence having about 98% sequence identity to SEQ ID NO: 66. In some embodiments, a secretion domain is encoded in a polynucleotide comprising a nucleotide sequence having about 99% sequence identity to SEQ ID NO: 66. In some embodiments, a secretion domain is encoded in a polynucleotide comprising a nucleotide sequence having about 100% sequence identity to SEQ ID NO: 66.

In some embodiments, a secretion domain is encoded in a polynucleotide comprising a nucleotide sequence as set forth in SEQ ID NO: 66. In some embodiments, a secretion domain is encoded in a polynucleotide consisting essentially of a nucleotide sequence as set forth in SEQ ID NO: 66. In some embodiments, a secretion domain is encoded in a polynucleotide consisting of a nucleotide sequence as set forth in SEQ ID NO: 66.

A polynucleotide encoding a 04469 secretion domain, as provided herein, may have more than one putative translation start site. Accordingly, a polynucleotide encoding a 04469 secretion domain may comprise a nucleotide sequence as provided in SEQ ID NO: 1, or a polynucleotide encoding a 04469 secretion domain may comprise a nucleotide sequence as provided in SEQ ID NO: 66 (which is identical to SEQ ID NO: 1, but includes 24 additional nucleotides at the 5′ end). When SEQ ID NO: 1 is translated, it produces a signaling peptide domain and a 04669 secretion domain comprising an amino acid sequence as shown in SEQ ID NO: 2. When SEQ ID NO: 66 is translated, it produces a signaling peptide domain and a 04669 secretion domain comprising an amino acid sequence as shown in SEQ ID NO: 67 (which is identical to SEQ ID NO: 2, but includes 8 additional amino acids at the N-terminus).

In some embodiments, a truncated 04469 secretion domain is used in accordance with the present disclosure. In some embodiments, a disclosed 04469 secretion domain is truncated at the C-terminus. In some embodiments, a 04469 secretion domain as disclosed in the present disclosure is truncated at the C-terminus by at least 1 amino acid, at least 2 amino acids, at least 3 amino acids, at least 4 amino acids, at least 5 amino acids, at least 6 amino acids, at least 7 amino acids, at least 8 amino acids, or more. In many embodiments, a 04469 secretion domain is truncated at the C-terminus by at least 5 amino acids. In some embodiments, a 04469 secretion domain is truncated at the C-terminus up to 1 amino acid, up to 2 amino acids, up to 3 amino acids, up to 4 amino acids, up to 5 amino acids, up to 6 amino acids, or up to 8 amino acids. In some embodiments, a 04469 secretion domain is truncated at the C-terminus up to 5 amino acids.

In some embodiments, a 04469 secretion domain as provided herein comprises a variant ribosomal binding site (RBS) that improves heterologous cargo secretion. In some embodiments, a recombinant fusion polypeptide as provided herein comprises a variant RBS as used in any one of SEQ ID NOs: 43-47. In some embodiments, a recombinant fusion polypeptide of the present disclosure comprises a 04469 secretion domain with a variant RBS and an N- or C-terminal linker such as a RAT linker, a (GS)4 linker, a PQP linker, or a NSQ linker, e.g., as used in the plasmids of SEQ ID NOs: 43-47.

### Heterologous Cargo Proteins

In some embodiments, a heterologous cargo protein of the present disclosure comprises an amino acid sequence of at least about 30 amino acids, at least about 40 amino acids, at least about 50 amino acids, at least about 60 amino acids, at least about 70 amino acids, at least about 80 amino acids, at least about 90 amino acids, at least about 100 amino acids, at least about 120 amino acids, at least about 140 amino acids, at least about 160 amino acids, at least about 180 amino acids, at least about 200 amino acids, at least about 220 amino acids, at least about 240 amino acids, at least about 260 amino acids, at least about 280 amino acids, at least about 300 amino acids, at least about 320 amino acids, at least about 340 amino acids, at least about 360 amino acids, at least about 380 amino acids, at least about 400 amino acids, at least about 450 amino acids, at least about 500 amino acids, at least about 600 amino acids, or at least about 700 amino acids.

In some embodiments, a heterologous cargo protein of the present disclosure comprises an amino acid sequence of up to about 30 amino acids, up to about 40 amino acids, up to about 50 amino acids, up to about 60 amino acids, up to about 70 amino acids, up to about 80 amino acids, up to about 90 amino acids, up to about 100 amino acids, up to about 120 amino acids, up to about 140 amino acids, up to about 160 amino acids, up to about 180 amino acids, up to about 200 amino acids, up to about 220 amino acids, up to about 240 amino acids, up to about 260 amino acids, up to about 280 amino acids, up to about 300 amino acids, up to about 320 amino acids, up to about 340 amino acids, up to about 360 amino acids, up to about 380 amino acids, up to about 400 amino acids, up to about 450 amino acids, up to about 500 amino acids, up to about 600 amino acids, or up to about 700 amino acids.

In some embodiments, a heterologous cargo protein of the present disclosure comprises an amino acid sequence of about 30 amino acids, about 40 amino acids, about 50 amino acids, about 60 amino acids, about 70 amino acids, about 80 amino acids, about 90 amino acids, about 100 amino acids, about 120 amino acids, about 140 amino acids, about 160 amino acids, about 180 amino acids, about 200 amino acids, about 220 amino acids, about 240 amino acids, about 260 amino acids, about 280 amino acids, about 300 amino acids, about 320 amino acids, about 340 amino acids, about 360 amino acids, about 380 amino acids, about 400 amino acids, about 450 amino acids, about 500 amino acids, about 600 amino acids, or about 700 amino acids.

In some embodiments, heterologous cargo proteins of the present disclosure comprise an amino acid sequence of about 2 to about 20 amino acids, about 2 to about 10 amino acids, about 10 to about 50 amino acids, about 10 to about 40 amino acids, about 10 to about 30 amino acids, about 10 to about 20 amino acids, about 20 to about 50 amino acids, about 30 to about 50 amino acids, about 40 to about 50 amino acids, about 40 to about 60 amino acids, about 50 to about 500 amino acids, about 50 to about 450 amino acids, about 50 to about 400 amino acids, about 50 to about 350 amino acids, about 50 to about 300 amino acids, about 50 to about 250 amino acids, about 50 to about 200 amino acids, about 50 to about 100 amino acids, about 100 to about 500 amino acids, about 200 to about 500 amino acids, about 250 to about 500 amino acids, about 300 to about 500 amino acids, about 400 to about 500 amino acids, about 420 to about 500 amino acids, about 460 to about 500 amino acids, about 480 to about 500 amino acids, about 450 to about 600 amino acids, about 500 to about 600 amino acids, or about 600 to about 700 amino acids.

In some embodiments, a heterologous cargo protein comprises a polypeptide of at least about 5 kDa, at least about 10 kDa, at least about 15 kDa, at least about 20 kDa, at least about 25 kDa, at least about 30 kDa, at least about 35 kDa, at least about 40 kDa, at least about 45 kDa, at least about 50 kDa, at least about 55 kDa, at least about 60 kDa, at least about 65 kDa, at least about 70 kDa, at least about 75 kDa, or at least about 80 kDa.

In some embodiments, a heterologous cargo protein comprises a polypeptide of up to about 5 kDa, up to about 10 kDa, up to about 15 kDa, up to about 20 kDa, up to about 25 kDa, up to about 30 kDa, up to about 35 kDa, up to about 40 kDa, up to about 45 kDa, up to about 50 kDa, up to about 55 kDa, up to about 60 kDa, up to about 65 kDa, up to about 70 kDa, up to about 75 kDa, or up to about 80 kDa.

In some embodiments, a heterologous cargo protein comprises a polypeptide of about 5 kDa, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, or about 80 kDa.

In some embodiments, a heterologous cargo protein comprises a polypeptide of about 5 to about 80 kDa, about 5 to about 70 kDa, about 5 to about 60 kDa, about 5 to about 50 kDa, about 5 to about 40 kDa, about 5 to about 30 kDa, about 5 to about 20 kDa, about 5 to about 10 kDa, about 10 to about 80 kDa, about 20 to about 80 kDa, about 30 to about 80 kDa, about 40 to about 80 kDa, about 50 to about 80 kDa, about 60 to about 80 kDa, about 70 to about 80 kDa, about 20 to about 70 kDa, about 30 to about 70 kDa, about 40 to about 70 kDa, about 50 to about 70 kDa, about 60 to about 70 kDa, about 20 to about 60 kDa, about 20 to about 50 kDa, about 20 to about 40 kDa, or about 20 to about 30 kDa.

In some embodiments, a heterologous cargo protein comprises a reporter polypeptide. In some embodiments, a reporter polypeptide is selected from the group consisting of: a luciferase (e.g., GFP, RFP, or YFP), horse radish peroxidase (HRP), beta-glucosidase, and variants or combinations thereof. In some embodiments, a reporter polypeptide comprises a luciferase. In some embodiments, a luciferase is a nanoluciferase.

In some embodiments, a heterologous cargo domain comprises a biologically active portion or fragment (e.g., a bioactive moiety, a therapeutic moiety, etc.). In some embodiments, a heterologous domain is a biologically active portion or fragment (e.g., a bioactive moiety, therapeutic moiety, etc.). In some embodiments, a heterologous cargo domain comprises a bioactive moiety. In some embodiments, a heterologous cargo domain comprises a therapeutic polypeptide. In some embodiments, a therapeutic polypeptide is or comprises a cytokine, hormone, antibody, affibody, nanobody, enzyme, bioactive peptide, or derivatives or functional fragments thereof. In some embodiments, a cytokine as provided by the instant disclosure may include IL-1, IL-2, IL-3, IL-4, IL-5, IL-6, IL-8, IL-9, IL-10, IL-11, IL-12, IL-13, IL-16, IL-18, IFN-alpha, IFN-beta, IFN-gamma, TNF-alpha, TNF-beta, or derivatives or functional fragments thereof, or combinations thereof.

In some embodiments, a heterologous cargo domain comprises a functional portion of an IL1Ra (e.g., as shown in SEQ ID NOs: 58 and 59), a TNFα affibody (e.g., as shown in SEQ ID NOs: 50 and 51), a VHH1-anti-TNF (e.g., as shown in SEQ ID NOs: 60 and 61), a MAM3 peptide (e.g., as shown in SEQ ID NOs: 56 and 57) and/or a HS2 detox protein (e.g., as shown in SEQ ID NOs: 52 and 53).

In some embodiments, a heterologous cargo domain comprises a Bacteroides protein that does not occur naturally in combination with a secretion domain as provided herein (e.g., 04469). In some embodiments, a Bacteroides protein is an immunomodulatory polypeptide or peptide.

### Linkers

As described herein, in some embodiments, a recombinant fusion polypeptide of the present disclosure may comprise one or more linker domains. In some embodiments, a linker domain comprises a glycine-serine linker sequence. In some embodiments, a linker domain comprises one or more glycine residues and/or one or more serine residues. In some embodiments, a linker domain comprises an polypeptide linker sequence of GGSGG (SEQ ID NO: 20). In some embodiments, a linker domain comprises a polypeptide linker sequence of (GGGGS)n (SEQ ID NO: 21), wherein “n” is a whole number integer, e.g., 1, 2, 3, 4, and so forth. In some embodiments, a linker domain comprises a RAT linker (including amino acid residues RAT). In some embodiments, a linker domain comprises a CL3 linker (SEQ ID NOs: 64 and 65). In some embodiments, a linker domain comprises a (GS)4 linker (SEQ ID NOs: 62 and 63). In some embodiments, a linker domain comprises a PQP linker (including amino acid residues PQP). In some embodiments, a linker domain comprises a NSQ linker (including amino acid residues NSQ). In some embodiments, a linker domain may comprise repeating linker sequences, or combinations of linker sequences, e.g., a linker domain may comprise two or more GGSGG (SEQ ID NO: 20) sequences, RAT linker sequences, (GS)4 linker sequences, PQP linker sequences, NSQ linker sequences, or combinations thereof.

In some embodiments, a linker domain comprises at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, or at least about 15 amino acids.

In some embodiments, a linker domain comprises up to about 2 amino acids, up to about 3 amino acids, up to about 4 amino acids, up to about 5 amino acids, up to about 6 amino acids, up to about 7 amino acids, up to about 8 amino acids, up to about 9 amino acids, up to about 10 amino acids, or up to about 15 amino acids.

In some embodiments, a linker domain comprises about 2 amino acids, about 3 amino acids, about 4 amino acids, about 5 amino acids, about 6 amino acids, about 7 amino acids, about 8 amino acids, about 9 amino acids, about 10 amino acids, or about 15 amino acids.

In some embodiments, a linker domain is a cleavable linker. In some embodiments, a cleavable linker comprises a protease cleavage site. In some embodiments, a protease cleavage site is cleaved by one or more proteases that are present in the colon of a subject. In some embodiments, a protease cleavage site is cleaved by a protease selected from the group consisting of: chymotrypsin, chymotrypsin-like elastases, trypsin, tobacco etch virus (TEV), thrombin, human neutrophil elastase, cathepsin G, tryptase, chymase, proteinase 3, commensal enteric microbial proteases, and combinations thereof.

### Epitope Tags

In some embodiments, a recombinant fusion polypeptide of the present disclosure comprises an epitope tag. In some embodiments, an epitope tag is also a reporter polypeptide. In some embodiments, an epitope tag is selected from the group consisting of: c-Myc, human influenza hemagglutinin (HA), FLAG, 3×FLAG, 6×His, glutathione-S-transferase (GST), DYKDDDDK, maltose binding protein (MBP), GFP, RFP, mCherry, or variants or combinations thereof. In some embodiments, an epitope tag is a 6×His tag. In some embodiments, an epitope tag is a 3×FLAG tag. In some embodiments, an epitope tag comprises a 6×His tag and a 3×FLAG tag.

### Polynucleotides and Vectors

### Polynucleotides

Another aspect of the disclosure relates to polynucleotides, including isolated polynucleotides, comprising a nucleotide sequence that encodes one or more engineered polypeptides as described herein (e.g., one or more recombinant fusion polypeptides as described herein). In some embodiments, a provided polynucleotide comprises one or more nucleic acid sequences as described herein. Such polynucleotides can be produced by recombinant DNA techniques known in the field. In some embodiments, a polynucleotide as provided herein is operably linked to a promoter. In some embodiments, a polynucleotide as provided herein is operably linked to a high expression promoter. In some embodiments, a polynucleotide as provided herein is operably linked to an inducible promoter, a repressible promoter, or a constitutive promoter. In some embodiments, a promoter is a native promoter (e.g., native to the bacterial cell). In some embodiments, a promoter is a heterologous promoter. In many embodiments, polynucleotides provided herein are present in a gene cassette or expression cassette, e.g., as described herein. In some embodiments, nucleotide sequences provided by the present disclosure are sequence optimized to provide a biological advantage, e.g., enhanced expression level, lower cell toxicity, etc.

### Vectors

Another aspect of the disclosure relates to a vector comprising a nucleic acid molecule as described herein. In one embodiment, a vector is an expression vector. In some embodiments, a vector is a plasmid as set forth in SEQ ID NO: 17. Such an expression vector comprises any nucleic acid molecule disclosed herein operably linked to a promoter to allow for expression of said nucleic acid molecule in a cell or cell-free extract. A wide variety of expression vectors can be employed for expressing a nucleic acid molecule encoding engineered polypeptides of the present disclosure (e.g., recombinant fusion polypeptides as described herein) including, without limitation, a viral expression vector; a prokaryotic expression vector; eukaryotic expression vectors, such as, e.g., a yeast expression vector, an insect expression vector and a mammalian expression vector; and a cell-free extract expression vector. It is further understood that expression vectors useful to practice aspects of these methods may include those which express engineered polypeptides of the present disclosure under control of a constitutive, tissue-specific, cell-specific or inducible promoter element, enhancer element or both. Expression vectors may include polynucleotides encoding protein tags, or epitope tags, to aid in isolation, purification or selection (e.g., poly-His tags, hemagglutinin tags, fluorescent protein tags, bioluminescent tags, and nuclear localization tags). As described herein, coding sequences for such protein tags can be fused to the coding sequences for provided engineered polypeptides (e.g., recombinant fusion polypeptides) or can be included in a separate expression cassette. Non-limiting examples of expression vectors, along with well-established reagents and conditions for making and using an expression construct from such expression vectors are readily available from commercial vendors that include, without limitation, BD Biosciences-Clontech, Palo Alto, Calif; BD Biosciences Pharmingen, San Diego, Calif.; Invitrogen, Inc, Carlsbad, Calif; EMD Biosciences-Novagen, Madison, Wis.; QIAGEN, Inc., Valencia, Calif.; and Stratagene, La Jolla, Calif. The selection, making and use of an appropriate expression vector are routine procedures well within the scope of one skilled in the art and from the teachings herein.

Vectors useful for transforming bacteria include plasmids, viruses (including phages), and integratable nucleic acid fragments (i.e., fragments integratable into the host genome by homologous recombination). Provided vectors may replicate and function independently of a host genome, or may, in some instances, integrate into a host genome. Suitable replicating vectors will contain a replicon and control sequences derived from species compatible with an intended expression host cell. In some embodiments, a polynucleotide encoding a gene of interest or gene cassette of interest (e.g., one that encodes for an engineered polypeptide of the present disclosure) is operably linked to an inducible promoter, a repressible promoter, or a constitutive promoter. One of ordinary skill in the art can determine the proper regulatory elements to use in a given vector for a given host cell. For a description of various components to be used in a representative commensal bacterium, Bacteroides thetaiotaomicron, see, e.g., as well as Mimee et al., Cell Syst. (2015) 1:62-71; Lim et al., Cell (2017) 169:547-558. See, also, Wang et al., Microb. Cell Fact. (2018) 17:63 (CRISPR-Cas9 editing of Corynebacterium glutamicum); Wang et al., ACS Synth. Biol. (2016) 15:721-732 (CRISPR-Cas9 editing of Clostridium beijerinckii); Xu et al., Appl. Environ. Microbial. (2015) (CRISPR-Cas9 editing of Clostridium cellulolylicum); Sun et al., Appl. Microbial. Biotech. (2015) 99:5151-5162 (GP35-meditated recombineering of Bacillus subtilis).

Vectors suitable for transforming bacteria are well known in the art. Typically, prokaryotic vectors comprise an origin of replication suitable for a target host cell (e.g., oriC derived from Escherichia coli, pUC derived from pBR322, pSC101 derived from Salmonella, 15A origin derived from p15A, and bacterial artificial chromosomes). Vectors can include a selectable marker (e.g., genes encoding resistance for ampicillin, chloramphenicol, gentamicin, and/or kanamycin). Zeocin™ (Life Technologies, Grand Island, NY) can be used as a selection marker in bacteria, fungi (including yeast), plants, and mammalian cell lines. Accordingly, vectors can be designed that carry only one drug resistance gene for Zeocin for selection work in a number of organisms. Useful promoters are known for expression of proteins in prokaryotes, for example, T5, T7, Rhamnose (inducible), Arabinose (inducible), and PhoA (inducible). Furthermore, T7 promoters are widely used in vectors that also encode a T7 RNA polymerase. Prokaryotic vectors can also include ribosome binding sites of varying strength and secretion signals (e.g., mal, sec, tat, ompC, and pelB). Prokaryotic RNA polymerase transcription termination sequences are also well known (e.g., transcription termination sequences from Streptococcus pyogenes).

General methods for construction of expression vectors are known in the art. Expression vectors for most host cells are commercially available. There are several commercial software products designed to facilitate selection of appropriate vectors and construction thereof, such as bacterial plasmids for bacterial transformation and gene expression in bacterial cells. Methods of introducing plasmids into host cells are known in the art and include, for example conjugation, bacteriophage infection, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery.

In some embodiments, a conjugation method is used to transform bacteria. See, e.g., Lederberg et al., Science (1953) 118: 169-175. The ability of bacteria to perform conjugation contributes to horizontal gene transfer and thus genome plasticity. In conjugations, plasmid DNA is transferred from one cell to another by a conjugative type IV secretion system (T4SS). See, e.g., Ilangovan et al., Trends Microbial. (2015) 23:301-310. Conjugative T4SS is a multiprotein secretion apparatus that is found in both gram-negative and gram-positive bacteria. Although the T4SS DNA transfer process in gram-negative and gram-positive organisms is similar, there are also differences that account for the differing physiology between the two main types of bacteria. The conjugation process can occur in three distinct steps. In the first and second steps, DNA is processed and recruited to the T4SS. In the third step, the processed DNA is translocated from one cell to another through the T4SS, which is one type of membrane secretion apparatus. Initiation of conjugation requires the formation of a multi protein-DNA complex, called the relaxosome, at the origin of transfer (oril). The enzyme relaxase, together with other accessory proteins, plays a crucial role in guiding the DNA through the T4SS to the recipient cell (see, e.g., Ilangovan et al., Trends Microbial. (2015) 23:301-310).

In gram-positive bacteria, there are distinct conjugative transfer mechanisms, which include the ability to transfer broad-host-range plasmids, e.g., pIP501, and Enterococcus sex pheromone-responsive plasmid, pCF10 (see, e.g., Goessweiner-Mohr et al., Microbial Spectr. (2014) 2:PLAS-0004-2013). Both plasmids mediate single-stranded DNA transfer in gram-positive bacteria. Alternatively, the conjugative transfer system found in Streptomyces mediates double-stranded DNA transfer.

In some embodiments of the present disclosure, polynucleotides for use herein are cloned into a bacteriophage (phage) specific for a bacterium of interest. Phages for use herein typically include a proteinaceous capsid and tail structure, which together serve to deliver genetic information to a targeted host cell. A phage capable of delivering DNA of non-phage origin to a bacterial cell is referred to as a transducing particle (TP). One of ordinary skill in the art can readily design TPs of specific DNA content such that a particular bacterium can be transduced with the TP. See, e.g., Chung et al., J Molec. Biol. (1990) 216:911-926; Chung et al., J Molec. Biol. (1990) 216:927-938. The process of generating a TP normally involves transferring the DNA packaging-related sequence elements of the phage to a plasmid. The phage DNA packaging machinery recognizes the plasmid as self and loads the non-self DNA into the capsid. The TP can then be used to deliver the plasmid to a target cell population.

Bacterial cells of interest can also be engineered using gene editing methods that utilize programmable nucleases that allow for targeted genetic modifications in a host cell genome by creating site-specific breaks at desired locations. Such nucleases include, but are not limited to, Clustered Regularly Interspaced Short Palindromic Repeats (CRISPR)-associated nucleases, zinc-finger nucleases (ZFNs), transcription activator-like effector nucleases (TALENs), and meganucleases. For reviews of these programmable nucleases see, e.g., Kim et al., Nature Reviews Genetics (2014) 15:321-334; Koonin et al., Curr Opin Microbial. (2017) 37:67-78; Makarova et al., Cell (2017) 168: 146; Makarova et al., Cell (2017) 168:328; Hsu et al., Cell (2014) 157: 1262-1278; Jore, et al., Nature Struc. and Molec. Biol. (2011) 18:529-536; Urnov et al., Nature Reviews Genetics (2010) 11:636-646; Stoddard, B., Mobile DNA (2014) 5:7; Joung et al., Nature Reviews Molecular Cell Biology (2013) 14:49-55. For example, a CRISPR guide polynucleotide can be used that preferentially targets a nucleic acid target sequence present in a genomic region in the organism to be modified. The CRISPR guide polynucleotide can be associated with a nucleic acid binding molecule, such as a DNA binding protein, that binds to and cleaves, the target sequence. A polynucleotide donor, e.g., a linear dsDNA donor, that contains a gene of interest and regions of homology to the targeted genetic region, or an expression cassette that includes the heterologous gene of interest and/or polynucleotide as described herein, can be delivered for insertion into the cleavage site by homologous recombination. CRISPR methods are well known in the art and are described in, e.g., Koonin et al., Curr Opin Microbial. (2017) 37:67-78; Makarova et al., Cell (2017) 168:146; Makarova et al., Cell (2017) 168:328; Hsu et al., Cell (2014) 157:1262-1278; Jore, et al., Nature Struc. and Molec. Biol. (2011) 18:529-536; Jinek, et al., Science (2012) 337:816-821; Briner et al., Molecular Cell (2014) 56:333-339; PCT Publication Nos. WO 2013/176772 (published 28 Nov. 2013) and WO 2014/150624 (published 25 Sep. 2014); and U.S. Pat. Nos. 9,580,701; 9,650,617; 9,688,972; 9,771,601; and 9,868,962.

In some cases, a genomic engineering procedure, such as lambda red recombineering, can be used to genetically modify a host cell genome. Lambda RED recombineering is based on the discovery and implementation of enzymes from the E. coli bacteriophage lambda and provides an efficient method of bacterial genome engineering (see, e.g., Court et al., Annual Review of Genetics (2002) 36:361-388). With lambda RED recombineering, cells are transformed by both a plasmid containing lambda RED recombination enzymes and linear double-stranded DNA (dsDNA) containing homology to the bacterial genome at the targeted genomic change. The lambda RED enzymes are exo, beta, and gam. Gam inhibits the endogenous recombination enzyme RecBCD that is also a highly potent and processive dsDNA exonuclease. Exo is a DNA exonuclease that generates single-stranded DNA (ssDNA) overhangs from the supplied linear dsDNA. Beta binds to ssDNA, and promotes strand invasion and homologous recombination (see, e.g., Court et al., Annual Review of Genetics (2002) 36:361-388; and Sawitzke et al., Methods Enzymol. (2013) 533: 157-177). Beta only requires 30-100 bases of homology for efficient recombination. Therefore, linear dsDNA for recombination can be generated by PCR with primers that contain homologous DNA. This technology can be coupled with the use of programmable nucleases, as described herein Typically, this requires the use of two plasmids and linear dsDNA. One plasmid encodes a programmable nuclease, and the other encodes a guide nucleic acid and the lambda RED enzymes. The linear dsDNA contains homology to the bacterial genome and the targeted genetic change. Each plasmid and the linear DNA are transformed into the bacteria sequentially. Other methods for genetically engineering bacteria include standard recombinant DNA techniques, well known in the art.

Bacteria that have been genetically engineered using a method as described herein, are then identified and isolated or purified using techniques well known in the art. For example, integration of an expression vector into a host bacterium can be confirmed by PCR analysis. Successful transformation can be confirmed, e.g., by isolating individual clones and screening the target locus using Sanger sequencing or analyzing cleavage efficiency using a restriction digest-based assay, such as a T7 endonuclease assay or a Surveyor assay. High-throughput screening techniques can also be employed, including, but not limited to, flow cytometry techniques such as fluorescence-activated cell sorting (FACS)-based screening platforms, microfluidics-based screening platforms, emulsion/droplet-based analysis methods, and the like. These techniques are well known in the art (see, e.g., Wojcik et al., Int. J Molec. Sci. (2015) 16:24918-24945).

In some embodiments, plasmids described herein can also contain sequences coding for a selectable marker or an antibiotic resistance gene, such that bacteria including the plasmids can be identified and isolated.

### Compositions

The present disclosure further provides for compositions of engineered Bacteroides cells that express engineered polypeptides, as described herein, thereby allowing for the secretion of heterologous proteins into a particular cellular environment. Accordingly, the engineered Bacteroides cells may function as a living cell protein delivery system suitable for delivering bioactive agents, therapeutic agents, reporters, and so forth. As Bacteroides cells are common in the gut of humans, the engineered Bacteroides cells are particularly suitable for delivering such payloads to the gastrointestinal tract of a subject. In many embodiments, an engineered polypeptide expressed by an engineered Bacteroides cell is a recombinant fusion polypeptide as described herein.

Engineered Bacteroides cells of the present disclosure may be produced using any of several methods known in the art, such as, but not limited to, standard recombinant DNA technology. For example, polynucleotides comprising nucleic acids that encode for any engineered polypeptide described herein can be delivered to a host Bacteroides cell using expression cassettes present in a vector (e.g., any vector as described herein).

In some embodiments, various cells as described herein can be for propagation of a nucleic acid, for expression of a nucleic acid, or both. Such cells for propagation include, without limitation, prokaryotic cells including, without limitation, strains of aerobic, microaerophilic, capnophilic, facultative, anaerobic, gram-negative and gram-positive bacterial cells such as those derived from, e.g., Escherichia coli, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca sexta; and mammalian cells and cell lines derived from mammalian cells, such as, e.g., those derived from mouse, rat, hamster, porcine, bovine, equine, primate and human.

Engineered Bacteroides cells, as described herein, may be produced using any species within the Bacteroides genus. In some embodiments, a provided engineered Bacteroides cell is a Bacteroides vulgatus, Bacteroides thetaiotaomicron, Bacteroides koreensis, Bacteroides graminisolvens, Bacteroides uniformis, Bacteroides ovatus, Bacteroides xylanisolvens, Bacteroides stercoris, Bacteroides finegoldii, Bacteroides cellulosilyticus, Bacteroides kribbi, Bacteroides fragilis, Bacteroides massiliensis, Bacteroides dorei, Bacteroides faecis, Bacteroides salyersiae, or Bacteroides caccae cell.

In some embodiments, a provided engineered Bacteroides cell comprises one or more transgenes that allow for utilization of a privileged nutrient as a carbon source or increase its ability to utilize a privileged nutrient as a carbon source. In some embodiments, a privileged nutrient is a marine polysaccharide. In some embodiments, a marine polysaccharide is porphyran or agarose. Engineered Bacteroides cells capable of utilizing certain privileged nutrient sources are described in PCT Publication WO 2018/112194 A1, the entire contents of which are incorporated herein in their entirety.

### Administration

Actual doses of provided compositions to be administered will vary depending upon the age, weight, and general condition of the subject as well as the severity of the condition being treated, the judgment of the health care professional, and particular cells and compositions being administered. Therapeutically effective amounts can be determined by one of ordinary skill in the art, and will be adjusted to the particular requirements of each particular case. Generally, a therapeutically effective amount for the use of live bacteria will be measured as colony forming units (CFU). Typically, a dose will include from about 1×10⁵ to about 1×10¹⁴ colony-forming units (CFU) of the engineered bacteria e.g., about 1×10⁶ to about 1×10¹¹; about 1×10⁷ to about 1×10¹⁰; about 1×10⁶ to about 1×10⁹, CFU of the genetically engineered bacteria; or any amount within the stated ranges. A therapeutically effective dose can be determined experimentally by repeated administration of increasing amounts of the composition in order to determine which amount produces a clinically desired endpoint.

Administration can be in a single bolus dose, or can be in two or more doses, in the same day, or one or more days apart. The amount of composition administered will depend on the potency of said composition, and/or route of administration.

The methods and compositions described herein can be used alone or in combination with other therapeutic agents and/or modalities. The term administered “in combination,” as used herein, is understood to mean that two (or more) different treatments are delivered to a subject during the course of said subject's affliction with a disease or disorder, such that the effects of each treatment on said subject overlap at a point in time. In some embodiments, delivery of one treatment is still occurring when delivery of a second begins, so that there is overlap in terms of administration. This is sometimes referred to herein as “simultaneous” or “concurrent delivery.” In some embodiments, delivery of one treatment ends before delivery of another treatment begins. In some embodiments of either case, a treatment is more effective because of combined administration.

For example, a second treatment may be more effective, e.g., an equivalent effect is seen with less of said second treatment, or a second treatment may reduce symptoms to a greater extent, than would be seen if said second treatment were administered in the absence of a first treatment, or the analogous situation is seen with a provided first treatment. In certain embodiments, delivery is such that reduction in one or more symptoms, or other parameters related to a disease or disorder is greater than what would be observed with one treatment delivered in the absence of the other. The effect of the two treatments can be partially additive, wholly additive, or greater than additive. The delivery can be such that an effect of a first treatment delivered is still detectable when a second is delivered.

In some embodiments, a method or composition described herein, is administered in combination with one or more additional therapies, e.g., anti-tumor agents, cell therapies, checkpoint inhibitors, GI disease or disorder therapies (e.g., treatments for irritable bowel syndrome (IBS), cancer (e.g., colorectal cancer), infectious colitis, ulcerative colitis, Crohn's disease, ischemic colitis, radiation colitis, peptic ulcer disease, gastritis, gastroenteritis, and celiac disease), and the like.

In some embodiments, method or compositions described herein can be administered in conjunction with H2-receptor antagonists (e.g., cimetidine, famotidine, nizatidine, ranitidine, etc.), proton pump inhibitors (e.g., esomeprazole, lansoprazole, omeprazole, pantoprazole, rabeprazole, etc.), antacids (e.g., aluminum hydroxide, magnesium carbonate, aluminum and magnesium complexes, etc.), alginates, chelates/complexes (e.g., tripotassium, dicitratobismuthate, sucralfate, etc.), and combinations thereof. In some embodiments, method or compositions described herein can be administered in conjunction with osmotics (e.g., polyethylene glycol (PEG 3350), such as Miralax@), stimulants (e.g., senna cascara, bisacodyl (such as Dulcolax®, Correctol®), etc.), magnesium based laxatives (e.g., milk of magnesia), anti-diarrheals (e.g., loperamide), secretagogues/prosecretory agents (e.g., lubiprostone, linaclotide, etc.), retainagogues (e.g., tenapanor), antispasmodics, anticholinergics (e.g., Levsin®, NuLev®, Levbid®, dicyclomine, etc.), direct smooth muscle relaxants (e.g., cimetropium, mebeverine, otilonium, pinaverium bromide, trimebutine, etc.), peppermint oil, tricyclic antidepressants (e.g., amitriptyline, notriptyline, imipramine, desipramine, etc.), selective serotonin reuptake inhibitors (SSRIs) (e.g., citalopram, fluoxetine, paroxetine, etc.), direct serotonin agonists/antagonists (e.g., tegaserod, alosetron, etc.), and combinations thereof. In some embodiments, method or compositions described herein can be administered in conjunction with Lialda, mesalamine, sulfasalazine, balsalazide, azathioprine, infliximab, vedolizumab, adalimumab, budesonide, olsalazine, golimumab, upadacitinib, ustekinumab, hydrocortisone, dexamethasone, Solu-Cortef, tofacitinib, olsalazine, ozanimod, or combinations thereof.

For example, various anti-cancer drugs are known and include, without limitation, alkylating agents (cisplatin, chlorambucil, procarbazine, carmustine, etc.), anti-metabolites (methotrexate, cytarabine, gemcitabine, etc.), anti-microtubule agents (vinblastine, paclitaxel, etc.), topoisomerase inhibitors (etoposide, doxorubicin, etc.), and cytotoxic agents (bleomycin, mitomycin, etc.). The appropriate chemotherapeutic drug to be used will depend on the type of disease or disorder being treated and the tolerance a patient has to a particular therapeutic agent.

Cell therapies can also be used in conjunction with provided genetically engineered bacteria, and include, but are not limited to, bone marrow transplants, stem cell transplants, and adoptive cell therapies (ACTs). ACTs use genetically modified adoptive cells derived from either a specific patient returned to that patient (autologous cell therapy) or from a third-party donor (allogeneic cell therapy) to treat the patient. ACTs, include, but are not limited to, T cell therapies, CAR-T cell therapies, and natural killer (NK) cell therapies. For example, T cells and NK cells can be modified to produce chimeric antigen receptors (CARs) on the T or NK cell surface (CAR-T cells and CAR-NK cells, respectively). These CAR-T cells recognize specific soluble antigens or antigens on a target cell surface, such as a tumor cell surface, or on cells in the tumor microenvironment. The CAR can comprise one or more extracellular ligand binding domains, a hinge region, a transmembrane region, and an intracellular signaling region. The extracellular ligand binding domain typically comprises a single-chain immunoglobulin variable fragment (scFv) or other ligand binding domain. The hinge region generally comprises a polypeptide hinge of variable length such as one or more amino acids, a CD8 alpha or an IgG4 region (or others), and combinations thereof. The transmembrane domain typically contains a transmembrane region derived from CD8 alpha, CD28, or other transmembrane proteins and combinations thereof. The intracellular signaling domain can consist of one or more intracellular signaling domains such as CD28, 4-1BB, CD3 zeta, OX40, or other intracellular signaling domains, and combinations thereof. When the extracellular ligand binding domain binds to a cognate ligand, the intracellular signaling domain of the CAR activates the lymphocyte. See, e.g., Brudno et al., Nature Rev. Clin. Oncol. (2018) 15:31-46; Maude et al., N. Engl. J. Med. (2014) 371:1507-1517; Sadelain et al., Cancer Disc. (2013) 3:388-398 (2018); U.S. Pat. Nos. 7,446,190 and 8,399,645 for descriptions of CAR-T cells, methods of making the same, and uses thereof. Examples of CAR-T approved therapies include e.g., the use of axicabtagene ciloleucel (Yescarta®; Kite Pharma, Inc., Foster City, CA), and tisagenlecleucel (Kymriah®; Novartis Pharmaceutical Corporation, Hanover, NJ).

In some embodiments, a method or composition described herein is administered in combination with a checkpoint inhibitor. A checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist. In some embodiments, a checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

In some embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910: 7,307,064, 7,109,003, 7,132,281, 6,207,156, 7,807,797, 7,824,679 8,143,379, 8,263,073, 8,318,916, 8,017,114, 8,784,815, and 8,883,984, International (PCT) Publication Nos. WO 98/42752, WO 00/37504, and WO 01/14424, and European Patent No. EP 1212422 B1. Exemplary CTLA-4 antibodies include ipilimumab or tremelimumab.

Checkpoint inhibitors such as those described herein can be used to treat various cancers, such as, but not limited to melanoma; lung cancer; kidney cancer; bladder cancer; stomach cancer; head and neck cancer; lymphomas, such as Hodgkin lymphoma; and solid tumors.

If the agents and therapies described above are provided at the same time as the engineered bacterial cells described herein, said cells can be provided in the same or in a different composition. Thus, engineered bacteria and other agents can be presented to an individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising genetically engineered organisms and a dose of a pharmaceutical composition comprising at least one other therapeutic agent, such as an anti-tumor agent, checkpoint inhibitor, and the like, which in combination comprises a therapeutically effective dose, according to a particular dosing regimen. Similarly, the genetically engineered microorganisms and therapeutic agents can be administered in at least one therapeutic dose. Administration of the separate pharmaceutical compositions can be performed simultaneously or at different times (e.g., sequentially, in either order, at the same time, or at different times), as long as the administration results in the therapeutic effect of the combination of these compositions.

Provided methods may further comprise administrating a privileged nutrient to the subject to support colonization of engineered bacterial cells of the present disclosure. Exemplary privileged nutrients include marine polysaccharides, e.g., a porphyran. For example, a disclosed privileged nutrient may be administered to the subject prior to, at the same time as, or after a disclosed bacterium.

Provided methods may comprise administration of a disclosed bacterium or pharmaceutical composition to a subject every 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. In some embodiments, the time between consecutive administrations of a disclosed bacterium or pharmaceutical composition to a subject is greater than 12 hours, 24 hours, 36 hours, 48 hours, 2 days, 3 days, 4 days, 5 days, 6 days, 7 days, 1 week, 2 weeks, 3 weeks, or 4 weeks.

Compositions of the present disclosure can be in the form of a liquid solution or suspension immediately prior to administration.

### Methods of Treating a Disease or Disorder

The present disclosure further provides methods useful for treating a disease or disorder in a subject. In some embodiments, methods of the present disclosure are useful for treating a disease or disorder in a subject via administration of Bacteroides cells that express a recombinant fusion polypeptide, e.g., as described herein. In particular, provided living cell secretion delivery systems can be used to treat any disease or disorder where delivery of an agent (e.g., a bioactive agent, therapeutic agent, etc.) to the GI tract of a subject is beneficial.

In some embodiments, provided methods of treating a disease or disorder comprise a step of administering to a subject an engineered Bacteroides cell, e.g., as described herein. In some embodiments, a disease or disorder is a gastrointestinal (GI) disease or disorder. In some embodiments, a GI disease or disorder is selected from the group consisting of: irritable bowel syndrome (IBS), cancer (e.g., colorectal cancer), infectious colitis, ulcerative colitis, Crohn's disease, ischemic colitis, radiation colitis, peptic ulcer disease, gastritis, gastroenteritis, and celiac disease. In some embodiments, a disease or disorder is a cancer. In some embodiments, a cancer is a gastric cancer. In some embodiments, a cancer is colorectal cancer. In some embodiments, a cancer is a pancreatic cancer.

In some embodiments, an administered engineered Bacteroides cell further comprises one or more transgenes that allow for utilization of a privileged nutrient as a carbon source or increase its ability to utilize a privileged nutrient as a carbon source (see, e.g., WO 2018/112194 A1). In some embodiments, a privileged nutrient is a marine polysaccharide. In some embodiments, a marine polysaccharide is porphyran or agarose. In many embodiments, a marine polysaccharide is a porphyran.

### Methods of Producing Engineered Polypeptides

The present disclosure provides for methods of producing a recombinant fusion polypeptide from an engineered Bacteroides cell. In some embodiments, the present disclosure provides a method comprising culturing a Bacteroides cell comprising a polynucleotide as described herein, wherein the encoded recombinant fusion polypeptide is secreted outside of the Bacteroides cell. In some embodiments, the method further comprises collecting (e.g., isolating or purifying) the recombinant fusion polypeptide. Collection of recombinant fusion polypeptide can be carried out by any method that is commonly known in the field, e.g., affinity-purification, column chromatography, precipitation, and combinations thereof.

### Methods of Quantifying Protein Secretion

The present disclosure also provides for methods and compositions for quantifying protein secretion from an engineered Bacteroides. In some embodiments, the present disclosure provides a method of quantifying protein secretion from an engineered Bacteroides comprising steps of: (i) culturing a Bacteroides cell capable of expressing a recombinant fusion polypeptide, e.g., as described herein; and (ii) measuring the amount of recombinant fusion polypeptide that is secreted outside of the Bacteroides cell. In some embodiments, a method comprises steps of: (i) administering to a subject a Bacteroides cell capable of expressing a recombinant fusion polypeptide, e.g., as described herein; and (ii) measuring the amount of recombinant fusion polypeptide present in the feces of the subject, thereby to quantify the protein secretion from the engineered Bacteroides cell. Measuring an amount of recombinant fusion polypeptide that is secreted outside of a cell or that is present in the feces of a subject can be carried out by any standard means known in the field. For example, amount of recombinant fusion polypeptide can be deduced by western blot, or any other immuno-detection methodologies (FACS, ELISA, etc.). Additionally, detection of nucleic acid sequences that encode all or part of a recombinant fusion polypeptide can be carried out by any known PCR-based technology.

In some embodiments of the present disclosure, at least about 70%, at least about 75%, at least about 80%, at least about 85%, at least about 90%, at least about 95%, or at least about 99% of recombinant fusion polypeptide is secreted as soluble protein. In some embodiments, about 70%, about 75%, about 80%, about 85%, about 90%, about 95%, about 99%, or about 100% of recombinant fusion polypeptide is secreted as soluble protein. In some embodiments, greater than about 70%, greater than about 75%, greater than about 80%, greater than about 85%, greater than about 90%, greater than about 95%, or greater than about 99% of recombinant fusion polypeptide is secreted as soluble protein. In some embodiments, secreted protein is not associated with outer membrane vesicles (OMVs).

### Methods of Assessing Colonization

Another aspect of the present disclosure relates to methods and compositions for assessing colonization by an engineered Bacteroides in the gut of a subject. In some embodiments, a method of assessing colonization by an engineered Bacteroides in the gut of a subject comprises steps of (i) administering to the subject a Bacteroides cell capable of expressing a recombinant fusion polypeptide, e.g., as described herein; and (ii) measuring the amount of recombinant fusion polypeptide present in the feces of the subject, thereby to assess colonization by the engineered Bacteroides in the gut of the subject. Measuring an amount of recombinant fusion polypeptide present in the feces of a subject can be carried out by any standard means known in the field. For example, amount of recombinant fusion polypeptide can be deduced by western blot, or any other immuno-detection methodologies (FACS, ELISA, etc.). Additionally, detection of nucleic acid sequences that encode all or part of a recombinant fusion polypeptide can be carried out by any known PCR-based technology.

Assessing colonization level of an engineered Bacteroides cell in the gut of a subject can be useful for adjusting dosage of a therapeutic regimen including said engineered Bacteroides cells, e.g., as described herein. For example, if colonization levels are lower than a particular benchmark, a therapeutic regimen can be modified to increase colonization to said benchmark. Conversely, if colonization levels are higher than a particular benchmark, a therapeutic regimen can be modified to decrease colonization to said benchmark. Such benchmarks, or optimal colonization levels, will be understood by a skilled medical practitioner.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As used in the present disclosure, the singular forms “a,” “an,” and “the” include plural referents unless the context clearly dictates otherwise. Thus, for example, “a gut microorganism” includes one or more such organisms.

The use of the alternative (e.g., “or”) should be understood to mean either one, both, or any combination thereof of the alternatives.

SEQUENCE LISTING

| | |
|---|---|
| SEQ ID NO: 104469 | ATGAAAAAAATGAGATTTTTATTAGTATTATTAGTGGGTAT |
| nucleotide sequence | ATCTATTGTATCATGTACTAATGACTTAGATGAAATGCTAG |
| (sequence before signal | GGACTCAAAATTTGCAAACCCGTTCTGTTGAGCCTAGATTC |
| peptide is cleaved in | ACAGGAAATATATCAGAAAGTCAGGCGATTGCATTAATGAA |
| periplasm) | TAAATTTCTGGATGCACCCTCTTTTTTTTATTTTGAAGATGTC |
| | ATAGGCTTCGATCCGACAACATCTATTTCCACAGGAAGTTT |
| | CACTGTCAACAGAAATGGGAAAACTATACATGTACGTTCTA |
| | TTCAAGTTGGTGTAATAGGAAAGAACGGAAAATATGGAGTT |
| | TATGGAACTCCAGTAACGAGCAAAGAATTCTATTCATTGAG |
| | AAATGGGATGGTTTTGCATGGAGAACCTATTTATGAAGAGA |
| | ATGATGTAGAAAGAGCCAACCCAGTAGGAATGCGTTATTAC |
| | TATACAACTAATGATGTAAATCATAGTCAAAATTGCTGGAT |
| | TGCGATCAATTGGGCTGATGCTTCTACATTTGAAAGTATAAT |
| | TGGTTGG |
| SEQ ID NO: 204469 | MKKMRFLLVLLVGISIVSCTNDLDEMLGTQNLQTRSVEPRFTG |
| amino acid sequence | NISESQAIALMNKFLDAPSFFYFEDVIGFDPTTSISTGSFTVNRN |
| (sequence before signal | GKTIHVRSIQVGVIGKNGKYGVYGTPVTSKEFYSLRNGMVLH |
| peptide is cleaved in | GEPIYEENDVERANPVGMRYYYTTNDVNHSQNCWIAINWAD |
| periplasm) | ASTFESIIGW |
| SEQ ID NO: 304469 | AATGACTTAGATGAAATGCTAGGGACTCAAAATTTGCAAAC |
| nucleotide sequence | CCGTTCTGTTGAGCCTAGATTCACAGGAAATATATCAGAAA |
| (sequence after signal | GTCAGGCGATTGCATTAATGAATAAATTTCTGGATGCACCC |
| peptide is cleaved in | TCTTTTTTTTATTTTGAAGATGTCATAGGCTTCGATCCGACA |
| periplasm) | ACATCTATTTCCACAGGAAGTTTCACTGTCAACAGAAATGG |
| | GAAAACTATACATGTACGTTCTATTCAAGTTGGTGTAATAG |
| | GAAAGAACGGAAAATATGGAGTTTATGGAACTCCAGTAAC |
| | GAGCAAAGAATTCTATTCATTGAGAAATGGGATGGTTTTGC |
| | ATGGAGAACCTATTTATGAAGAGAATGATGTAGAAAGAGC |
| | CAACCCAGTAGGAATGCGTTATTACTATACAACTAATGATG |
| | TAAATCATAGTCAAAATTGCTGGATTGCGATCAATTGGGCT |
| | GATGCTTCTACATTTGAAAGTATAATTGGTTGG |
| SEQ ID NO: 404469 | NDLDEMLGTQNLQTRSVEPRFTGNISESQAIALMNKFLDAPSFF |
| amino acid sequence | YFEDVIGFDPTTSISTGSFTVNRNGKTIHVRSIQVGVIGKNGKY |
| (sequence after signal | GVYGTPVTSKEFYSLRNGMVLHGEPIYEENDVERANPVGMRY |
| peptide is cleaved in | YYTTNDVNHSQNCWIAINWADASTFESIIGW |
| periplasm) | |
| SEQ ID NO: 5- | ATGTTAAATTTAAATTATGTAAAGATGAAAAAAATGAGATT |
| 04469-″an 8 residue | TTTATTAGTATTATTAGTGGGTATATCTATTGTATCATGTAC |
| glycine serine linker″- | TAATGACTTAGATGAAATGCTAGGGACTCAAAATTTGCAAA |
| NL nucleotide | CCCGTTCTGTTGAGCCTAGATTCACAGGAAATATATCAGAA |
| sequence | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCTAGTGTTTTTACTCTGGAAGATTTTGTTG |
| | GCGATTGGCGTCAGACCGCGGGTTATAATTTGGATCAAGTC |
| | CTGGAACAGGGTGGCGTAAGCTCTCTGTTCCAGAACCTGGG |
| | TGTGAGCGTGACGCCGATTCAGCGCATCGTTCTGTCCGGCG |
| | AGAACGGTCTGAAAATTGATATTCATGTGATCATCCCGTAC |
| | GAAGGCCTGAGCGGTGACCAAATGGGTCAAATCGAGAAAA |
| | TCTTTAAAGTCGTCTACCCAGTTGACGATCACCACTTCAAGG |
| | TTATCTTGCATTACGGTACGCTGGTGATTGATGGTGTGACCC |
| | CGAATATGATTGACTATTTCGGCCGTCCGTATGAAGGCATT |
| | GCCGTTTTTGACGGTAAAAAGATCACCGTCACCGGTACCCT |
| | GTGGAATGGCAATAAGATTATTGACGAGCGTCTGATTAACC |
| | CGGACGGCAGCCTGCTGTTCCGCGTGACCATCAACGGTGTC |
| | ACGGGTTGGCGTCTGTGCGAGCGCATCCTGGCATAA |
| SEQ ID NO: 6- | MLNLNYVKMKKMRFLLVLLVGISIVSCTNDLDEMLGTQNLQT |
| 04469-″an 8 residue | RSVEPRFTGNISESQAIALMNKFLDAPSFFYFEDVIGFDPTTSIST |
| glycine serine linker″- | GSFTVNRNGKTIHVRSIQVGVIGKNGKYGVYGTPVTSKEFYSL |
| NL amino acid | RNGMVLHGEPIYEENDVERANPVGMRYYYTTNDVNHSQNCW |
| sequence | IAINWADASTFESIIGWGGSGGGSSVFTLEDFVGDWRQTAGYN |
| | LDQVLEQGGVSSLFQNLGVSVTPIQRIVLSGENGLKIDIHVIIPY |
| | EGLSGDQMGQIEKIFKVVYPVDDHHFKVILHYGTLVIDGVTPN |
| | MIDYFGRPYEGIAVFDGKKITVTGTLWNGNKIIDERLINPDGSL |
| | LFRVTINGVTGWRLCERILA |
| SEQ ID NO: 7- | ATGTTAAATTTAAATTATGTAAAGATGAAAAAAATGAGATT |
| 04469-3XFLAG- | TTTATTAGTATTATTAGTGGGTATATCTATTGTATCATGTAC |
| 6XHis nucleotide | TAATGACTTAGATGAAATGCTAGGGACTCAAAATTTGCAAA |
| sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAATGG |
| | ACTACAAGGATCATGATGGGGATTACAAGGACCATGACATA |
| | GATTACAAAGATGATGATGATAAAGGGTCTAGTGATTACAA |
| | GGACGATGATGATAAGGACTACAAAGATGATGATGACAAA |
| | GATTATAAGGACGATGATGATAAAGGTCATCATCATCATCA |
| | TCACTAA |
| SEQ ID NO: 8- | MLNLNYVKMKKMRFLLVLLVGISIVSCTNDLDEMLGTQNLQT |
| 04469-3XFLAG- | RSVEPRFTGNISESQAIALMNKFLDAPSFFYFEDVIGFDPTTSIST |
| 6XHis amino acid | GSFTVNRNGKTIHVRSIQVGVIGKNGKYGVYGTPVTSKEFYSL |
| sequence | RNGMVLHGEPIYEENDVERANPVGMRYYYTTNDVNHSQNCW |
| | IAINWADASTFESIIGWGMDYKDHDGDYKDHDIDYKDDDDKG |
| | SSDYKDDDDKDYKDDDDKDYKDDDDKGHHHHHH |
| SEQ ID NO: 9- | ATGTTAAATTTAAATTATGTAAAGATGAAAAAAATGAGATT |
| 04469-NL ″full length″ | TTTATTAGTATTATTAGTGGGTATATCTATTGTATCATGTAC |
| from sWW3378 | TAATGACTTAGATGAAATGCTAGGGACTCAAAATTTGCAAA |
| nucleotide sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCACCGACAGCTTCTCCTCTGGGTTCCGGT |
| | GGCTCTGGTGGTGTTTTTACTCTGGAAGATTTTGTTGGCGAT |
| | TGGCGTCAGACCGCGGGTTATAATTTGGATCAAGTCCTGGA |
| | ACAGGGTGGCGTAAGCTCTCTGTTCCAGAACCTGGGTGTGA |
| | GCGTGACGCCGATTCAGCGCATCGTTCTGTCCGGCGAGAAC |
| | GGTCTGAAAATTGATATTCATGTGATCATCCCGTACGAAGG |
| | CCTGAGCGGTGACCAAATGGGTCAAATCGAGAAAATCTTTA |
| | AAGTCGTCTACCCAGTTGACGATCACCACTTCAAGGTTATCT |
| | TGCATTACGGTACGCTGGTGATTGATGGTGTGACCCCGAAT |
| | ATGATTGACTATTTCGGCCGTCCGTATGAAGGCATTGCCGTT |
| | TTTGACGGTAAAAAGATCACCGTCACCGGTACCCTGTGGAA |
| | TGGCAATAAGATTATTGACGAGCGTCTGATTAACCCGGACG |
| | GCAGCCTGCTGTTCCGCGTGACCATCAACGGTGTCACGGGT |
| | TGGCGTCTGTGCGAGCGCATCCTGGCATAA |
| SEQ ID NO: 10- | MLNLNYVKMKKMRFLLVLLVGISIVSCTNDLDEMLGTQNLQT |
| 04469-NL ″full length″ | RSVEPRFTGNISESQAIALMNKFLDAPSFFYFEDVIGFDPTTSIST |
| from sWW3378 amino | GSFTVNRNGKTIHVRSIQVGVIGKNGKYGVYGTPVTSKEFYSL |
| acid sequence | RNGMVLHGEPIYEENDVERANPVGMRYYYTTNDVNHSQNCW |
| | IAINWADASTFESIIGWGGSGGGSPTASPLGSGGSGGVFTLEDF |
| | VGDWRQTAGYNLDQVLEQGGVSSLFQNLGVSVTPIQRIVLSG |
| | ENGLKIDIHVIIPYEGLSGDQMGQIEKIFKVVYPVDDHHFKVIL |
| | HYGTLVIDGVTPNMIDYFGRPYEGIAVFDGKKITVTGTLWNGN |
| | KIIDERLINPDGSLLFRVTINGVTGWRLCERILA |
| SEQ ID NO: 11- | ATGTTAAATTTAAATTATGTAAAGATGAAAAAAATGAGATT |
| 04469-″delta 5″-NL | TTTATTAGTATTATTAGTGGGTATATCTATTGTATCATGTAC |
| nucleotide sequence | TAATGACTTAGATGAAATGCTAGGGACTCAAAATTTGCAAA |
| | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAGGAGGATCCGGCGGAGGGTCAC |
| | CGACAGCTTCTCCTCTGGGTTCCGGTGGCTCTGGTGGTGTTT |
| | TTACTCTGGAAGATTTTGTTGGCGATTGGCGTCAGACCGCG |
| | GGTTATAATTTGGATCAAGTCCTGGAACAGGGTGGCGTAAG |
| | CTCTCTGTTCCAGAACCTGGGTGTGAGCGTGACGCCGATTC |
| | AGCGCATCGTTCTGTCCGGCGAGAACGGTCTGAAAATTGAT |
| | ATTCATGTGATCATCCCGTACGAAGGCCTGAGCGGTGACCA |
| | AATGGGTCAAATCGAGAAAATCTTTAAAGTCGTCTACCCAG |
| | TTGACGATCACCACTTCAAGGTTATCTTGCATTACGGTACGC |
| | TGGTGATTGATGGTGTGACCCCGAATATGATTGACTATTTCG |
| | GCCGTCCGTATGAAGGCATTGCCGTTTTTGACGGTAAAAAG |
| | ATCACCGTCACCGGTACCCTGTGGAATGGCAATAAGATTAT |
| | TGACGAGCGTCTGATTAACCCGGACGGCAGCCTGCTGTTCC |
| | GCGTGACCATCAACGGTGTCACGGGTTGGCGTCTGTGCGAG |
| | CGCATCCTGGCATAA |
| SEQ ID NO: 12- | MLNLNYVKMKKMRFLLVLLVGISIVSCTNDLDEMLGTQNLQT |
| 04469-″delta 5″-NL | RSVEPRFTGNISESQAIALMNKFLDAPSFFYFEDVIGFDPTTSIST |
| amino acid sequence | GSFTVNRNGKTIHVRSIQVGVIGKNGKYGVYGTPVTSKEFYSL |
| | RNGMVLHGEPIYEENDVERANPVGMRYYYTTNDVNHSQNCW |
| | IAINWADASTFEGGSGGGSPTASPLGSGGSGGVFTLEDFVGDW |
| | RQTAGYNLDQVLEQGGVSSLFQNLGVSVTPIQRIVLSGENGLK |
| | IDIHVIIPYEGLSGDQMGQIEKIFKVVYPVDDHHFKVILHYGTL |
| | VIDGVTPNMIDYFGRPYEGIAVFDGKKITVTGTLWNGNKIIDER |
| | LINPDGSLLFRVTINGVTGWRLCERILA |
| SEQ ID NO: 13- | ATGTTAAATTTAAATTATGTAAAGATGAAAAAAATGAGATT |
| 04469-″delta 8″-NL | TTTATTAGTATTATTAGTGGGTATATCTATTGTATCATGTAC |
| nucleotide sequence | TAATGACTTAGATGAAATGCTAGGGACTCAAAATTTGCAAA |
| | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTGGAGGATCCGGCGGAGGGTCACCGACAGCTT |
| | CTCCTCTGGGTTCCGGTGGCTCTGGTGGTGTTTTTACTCTGG |
| | AAGATTTTGTTGGCGATTGGCGTCAGACCGCGGGTTATAAT |
| | TTGGATCAAGTCCTGGAACAGGGTGGCGTAAGCTCTCTGTT |
| | CCAGAACCTGGGTGTGAGCGTGACGCCGATTCAGCGCATCG |
| | TTCTGTCCGGCGAGAACGGTCTGAAAATTGATATTCATGTG |
| | ATCATCCCGTACGAAGGCCTGAGCGGTGACCAAATGGGTCA |
| | AATCGAGAAAATCTTTAAAGTCGTCTACCCAGTTGACGATC |
| | ACCACTTCAAGGTTATCTTGCATTACGGTACGCTGGTGATTG |
| | ATGGTGTGACCCCGAATATGATTGACTATTTCGGCCGTCCGT |
| | ATGAAGGCATTGCCGTTTTTGACGGTAAAAAGATCACCGTC |
| | ACCGGTACCCTGTGGAATGGCAATAAGATTATTGACGAGCG |
| | TCTGATTAACCCGGACGGCAGCCTGCTGTTCCGCGTGACCA |
| | TCAACGGTGTCACGGGTTGGCGTCTGTGCGAGCGCATCCTG |
| | GCATAA |
| SEQ ID NO: 14- | MLNLNYVKMKKMRFLLVLLVGISIVSCTNDLDEMLGTQNLQT |
| 04469-″delta 8″-NL | RSVEPRFTGNISESQAIALMNKFLDAPSFFYFEDVIGFDPTTSIST |
| amino acid sequence | GSFTVNRNGKTIHVRSIQVGVIGKNGKYGVYGTPVTSKEFYSL |
| | RNGMVLHGEPIYEENDVERANPVGMRYYYTTNDVNHSQNCW |
| | IAINWADASGGSGGGSPTASPLGSGGSGGVFTLEDFVGDWRQT |
| | AGYNLDQVLEQGGVSSLFQNLGVSVTPIQRIVLSGENGLKIDIH |
| | VIIPYEGLSGDQMGQIEKIFKVVYPVDDHHFKVILHYGTLVIDG |
| | VTPNMIDYFGRPYEGIAVFDGKKITVTGTLWNGNKIIDERLINP |
| | DGSLLFRVTINGVTGWRLCERILA |
| SEQ ID NO: 15- | ATGTTAAATTTAAATTATGTAAAGATGAAAAAAATGAGATT |
| 04469-″delta 12″-NL | TTTATTAGTATTATTAGTGGGTATATCTATTGTATCATGTAC |
| nucleotide sequence | TAATGACTTAGATGAAATGCTAGGGACTCAAAATTTGCAAA |
| | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGG |
| | AGGATCCGGCGGAGGGTCACCGACAGCTTCTCCTCTGGGTT |
| | CCGGTGGCTCTGGTGGTGTTTTTACTCTGGAAGATTTTGTTG |
| | GCGATTGGCGTCAGACCGCGGGTTATAATTTGGATCAAGTC |
| | CTGGAACAGGGTGGCGTAAGCTCTCTGTTCCAGAACCTGGG |
| | TGTGAGCGTGACGCCGATTCAGCGCATCGTTCTGTCCGGCG |
| | AGAACGGTCTGAAAATTGATATTCATGTGATCATCCCGTAC |
| | GAAGGCCTGAGCGGTGACCAAATGGGTCAAATCGAGAAAA |
| | TCTTTAAAGTCGTCTACCCAGTTGACGATCACCACTTCAAGG |
| | TTATCTTGCATTACGGTACGCTGGTGATTGATGGTGTGACCC |
| | CGAATATGATTGACTATTTCGGCCGTCCGTATGAAGGCATT |
| | GCCGTTTTTGACGGTAAAAAGATCACCGTCACCGGTACCCT |
| | GTGGAATGGCAATAAGATTATTGACGAGCGTCTGATTAACC |
| | CGGACGGCAGCCTGCTGTTCCGCGTGACCATCAACGGTGTC |
| | ACGGGTTGGCGTCTGTGCGAGCGCATCCTGGCATAA |
| SEQ ID NO: 16- | MLNLNYVKMKKMRFLLVLLVGISIVSCTNDLDEMLGTQNLQT |
| 04469-″delta 12″-NL | RSVEPRFTGNISESQAIALMNKFLDAPSFFYFEDVIGFDPTTSIST |
| amino acid sequence | GSFTVNRNGKTIHVRSIQVGVIGKNGKYGVYGTPVTSKEFYSL |
| | RNGMVLHGEPIYEENDVERANPVGMRYYYTTNDVNHSQNCW |
| | IAINWGGSGGGSPTASPLGSGGSGGVFTLEDFVGDWRQTAGY |
| | NLDQVLEQGGVSSLFQNLGVSVTPIQRIVLSGENGLKIDIHVIIP |
| | YEGLSGDQMGQIEKIFKVVYPVDDHHFKVILHYGTLVIDGVTP |
| | NMIDYFGRPYEGIAVFDGKKITVTGTLWNGNKIIDERLINPDGS |
| | LLFRVTINGVTGWRLCERILA |
| SEQ ID NO: 17- | GATAAAACGAAAGGCTCAGTCGAAAGACTGGGCCTTTCGTT |
| BfP1E6- | TTACAATTGGGCTACCTTTTTTTTGTTTTGTTTGCAATGGTTA |
| 04469_P5017- | ATCTATTGTTAAAATTTAAAGTTTCACTTGAACTTTCAAATA |
| GGSGG-RAT- | ATGTTCTTATATTTGCAGTGTCGAAAGAAACAAAGTAGCCT |
| NanoLuc-GSGSGSGS! | GGATCTACTTTTAAAATTTATAAAATGTTGAATTTAAATTAT |
| nucleotide sequence | GTGAAAATGAAGAAAATGAGATTTCTATTAGTATTATTGGT |
| | CGGGATATCTATTGTATCATGTACTAATGACTTAGATGAAA |
| | TGCTAGGGACTCAAAATTTGCAAACCCGTTCTGTTGAGCCT |
| | AGATTCACAGGAAATATATCAGAAAGTCAGGCGATTGCATT |
| | AATGAATAAATTTCTGGATGCACCCTCTTTTTTTTATTTTGA |
| | AGATGTCATAGGCTTCGATCCGACAACATCTATTTCCACAG |
| | GAAGTTTCACTGTCAACAGAAATGGGAAAACTATACATGTA |
| | CGTTCTATTCAAGTTGGTGTAATAGGAAAGAACGGAAAATA |
| | TGGAGTTTATGGAACTCCAGTAACGAGCAAAGAATTCTATT |
| | CATTGAGAAATGGGATGGTTTTGCATGGAGAACCTATTTAT |
| | GAAGAGAATGATGTAGAAAGAGCCAACCCAGTAGGAATGC |
| | GTTATTACTATACAACTAATGATGTAAATCATAGTCAAAAT |
| | TGCTGGATTGCGATCAATTGGGCTGATGCTTCTACATTTGAA |
| | AGTATAATTGGTTGGGGAGGATCCGGCGGAGGGTCACGTGC |
| | GACGGTTTTTACTCTGGAAGATTTTGTTGGCGATTGGCGTCA |
| | GACCGCGGGTTATAATTTGGATCAAGTCCTGGAACAGGGTG |
| | GCGTAAGCTCTCTGTTCCAGAACCTGGGTGTGAGCGTGACG |
| | CCGATTCAGCGCATCGTTCTGTCCGGCGAGAACGGTCTGAA |
| | AATTGATATTCATGTGATCATCCCGTACGAAGGCCTGAGCG |
| | GTGACCAAATGGGTCAAATCGAGAAAATCTTTAAAGTCGTC |
| | TACCCAGTTGACGATCACCACTTCAAGGTTATCTTGCATTAC |
| | GGTACGCTGGTGATTGATGGTGTGACCCCGAATATGATTGA |
| | CTATTTCGGCCGTCCGTATGAAGGCATTGCCGTTTTTGACGG |
| | TAAAAAGATCACCGTCACCGGTACCCTGTGGAATGGCAATA |
| | AGATTATTGACGAGCGTCTGATTAACCCGGACGGCAGCCTG |
| | CTGTTCCGCGTGACCATCAACGGTGTCACGGGTTGGCGTCT |
| | GTGCGAGCGCATCCTGGCAGGTTCCGGGTCTGGATCAGGTA |
| | GCTAAGGTTCCTAAGGCCATCCTGACGGATGGCCTTTTTTTT |
| | GACTGCTATGACTTGATACCGGCTATTACGAGCGCTTAAAC |
| | GGCGCGCCTGATAGGTGGGCTGCCCTTCCTGGTTGGCTTGG |
| | TTTCATCAGCCATCCGCTTGCCCTCATCTGTTACGCCGGCGG |
| | TAGCCGGCCAGCCTCGCAGAGCAGGATTCCCGTTGAGCACC |
| | GCCAGGTGCGAATAAGGGACAGTGAAGAAGGAACACCCGC |
| | TCGCGGGTGGGCCTACTTCACCTATCCTGCCCGGCTGACGC |
| | CGTTGGATACACCAAGGAAAGTCTACACGAACCCTTTGGCA |
| | AAATCCTGTATATCGTGCGAAAAAGGATGGATATACCGAAA |
| | AAATCGCTATAATGACCCCGAAGCAGGGTTATGCAGCGGAA |
| | AAGCGGGATTAAAAGTCGGGGATTGGTGAACAAAAAGGTG |
| | TTTCTCTCTTTAAGAGAAATATCGTTTTGCTAAACAGTTGAT |
| | ATTGAGGTATCATTTTATCGTAAAAGACATTTTTGCTCAACA |
| | ATTGCTTGACGGAAATCAACAAATTTTAGCATTTTGTAAAA |
| | AAGTCGCTATATAATTTGGTGAATTGGAGTTATTTTCATATT |
| | TTTGCATCCCGAAGAGTTTCTCTTAAAGAGAGAAACATCTTT |
| | TGCATACCTTTTCCGACCGAATTTTTATGTCGTAAAGAGGGG |
| | CTTTGCAGGGGGTGGACTCAGAAAGATGAGAATAGATGACT |
| | ATTGTAGTTGAAACACATAGAAAGTTGCTGATATACAGACC |
| | GATACGCATATCGGGATGAACCATGAGTACGTTCTTTTCTC |
| | AAAAAACATAAATATTCGAAAAGAGATGCAATAAATTAAG |
| | GAGAGGTTATAATGAACAAAGTAAATATAAAAGATAGTCA |
| | AAATTTTATTACTTCAAAATATCACATAGAAAAAATAATGA |
| | ATTGCATAAGTTTAGATGAAAAAGATAACATCTTTGAAATA |
| | GGTGCAGGGAAAGGTCATTTTACTGCTGGATTGGTAAAGAG |
| | ATGTAATTTTGTAACGGCGATAGAAATTGATTCTAAATTAT |
| | GTGAGGTAACTCGTAATAAGCTCTTAAATTATCCTAACTATC |
| | AAATAGTAAATGATGATATACTGAAATTTACATTTCCTAGC |
| | CACAATCCATATAAAATATTTGGCAGCATACCTTACAACAT |
| | AAGCACAAATATAATTCGAAAAATTGTTTTTGAAAGTTCAG |
| | CCACAATAAGTTATTTAATAGTGGAATATGGTTTTGCTAAA |
| | ATGTTATTAGATACAAACAGATCACTAGCATTGCTGTTAAT |
| | GGCAGAGGTAGATATTTCTATATTAGCAAAAATTCCTAGGT |
| | ATTATTTCCATCCAAAACCTAAAGTGGATAGCACATTAATT |
| | GTATTAAAAAGAAAGCCAGCAAAAATGGCATTTAAAGAGA |
| | GAAAAAAATATGAAACTTTTGTAATGAAATGGGTTAACAAA |
| | GAGTACGAAAAACTGTTTACAAAAAATCAATTTAATAAAGC |
| | TTTAAAACATGCGAGAATATATGATATAAACAATATTAGTT |
| | TCGAACAATTTGTATCGCTATTTAATAGTTATAAAATATTTA |
| | ACGGCTAAAAACAATAGGCCACATGCAACTGTAAATGTTTA |
| | CGCGGGTACCGACACCGCGGTGGAGGGGAATTGTGTTACAA |
| | CCAATTAACCAATTCTGATTAGAAAAACTCATCGAGCATCA |
| | AATGAAACTGCAATTTATTCATATCAGGATTATCAATACCA |
| | TATTTTTGAAAAAGCCGTTTCTGTAATGAAGGAGAAAACTC |
| | ACCGAGGCAGTTCCATAGGATGGCAAGATCCTGGTATCGGT |
| | CTGCGATTCCGACTCGTCCAACATCAATACAACCTATTAATT |
| | TCCCCTCGTCAAAAATAAGGTTATCAAGTGAGAAATCACCA |
| | TGAGTGACGACTGAATCCGGTGAGAATGGCAAAAGCTTATG |
| | CATTTCTTTCCAGACTTGTTCAACAGGCCAGCCATTACGCTC |
| | GTCATCAAAATCACTCGCATCAACCAAACCGTTATTCATTC |
| | GTGATTGCGCCTGAGCGAGGCGAAATACGCGATCGCTGTTA |
| | AAAGGACAATTACAAACAGGAATCGAATGCAACCGGCGCA |
| | GGAACACTGCCAGCGCATCAACAATATTTTCACCTGAATCA |
| | GGATATTCTTCTAATACCTGGAATGCTGTTTTCCCGGGGATC |
| | GCAGTGGTGAGTAACCATGCATCATCAGGAGTACGGATAAA |
| | ATGCTTGATGGTCGGAAGAGGCATAAATTCCGTCAGCCAGT |
| | TTAGTCTGACCATCTCATCTGTAACATCATTGGCAACGCTAC |
| | CTTTGCCATGTTTCAGAAACAACTCTGGCGCATCGGGCTTCC |
| | CATACAATCGATAGATTGTCGCACCTGATTGCCCGACATTA |
| | TCGCGAGCCCATTTATACCCATATAAATCAGCATCCATGTTG |
| | GAATTTAATCGCGGCCTGGAGCAAGACGTTTCCCGTTGAAT |
| | ATGGCTCATAACACCCCTTGTATTACTGTTTATGTAAGCAGA |
| | CAGTTTTATTGTTCATGATGATATATTTTTATCTTGTGCAAT |
| | GTAACATCAGAGATTTTGAGACACAACGTGGCTTTGTTGAA |
| | TAAATCGAACTTTTGCTGAGTTGAAGGATCAGGTCATTGGT |
| | AACTATCTATGAAACTGTTTGATACTTTTATAGTTGATTAAA |
| | CTTGTTCATGGCATTTGCCTTAATATCATCCGCTATGTCAAT |
| | GTAGGGTTTCATAGCTTTGTAGTCGCTGTGTCCCGTCCATTT |
| | CATGACCACCTGTGCCGGGATTCCGAGAGCCAGCGCATTGC |
| | AGATGAATGTCCTTTTTCCTGCATGGGTACTGAGCAAAGCG |
| | TATTTGGGTGTGACTTCATCAATACGTTCATTTCCCTTGTAG |
| | TAGGTTTCCCGTACAGGCTCGTTGATTTCTGCCAGTTCGCCC |
| | AGCTCTTTCAGGTAATCGTTCATCTTCTGGTTGCTGATGACG |
| | GGCAGAGCCATGTAATTCTCGAAATGGATGTCCTTGTATTT |
| | GTCCAGTATGGCTTTGCTGTATTTGTTCAGTTCAATCGTCAG |
| | GCTGTCGGCAGTCTTGACTGTGGTTATTTCGATGTGGTCGGA |
| | CTTCACATCGCTTCTTTTCAGATTGCGAACATCCGAATACCG |
| | CAAACTCGTAAAGCAGCAGAACAGGAAAACATCACGCACA |
| | CGTTCCAGGTATTGCTTATCCTTGGGTATCTGGTAGTCTTTC |
| | AGCTTGTTCAGTTCATCCCAAGTCAGGAAGATTACTTTTTTC |
| | GAGGTGGTTTTCAGTTTCGGTTTGAACGTATCGTATGCAATG |
| | TTCTGATGATGTCCTTTCTTGAAGCTCCAGCGCAGGAACCAT |
| | TTGAGGAATCCCATTTGCTTGCCGATGGTGCTGTTTCTCATA |
| | TCCTTGGTGTCACGCAGGAAGTTGACGTATTCGTTCAATCCA |
| | AACTCGTTGAAATAGTTGAACGTTGCATCCTCCTTGAACTCT |
| | TTGAGGTGGTTCCTCACTGCTGCAAATTTTTCATAGGTGGAT |
| | GCCGTCCAGTTATTCTGGTTACCGCACTCTTTTACAAACTCA |
| | TCGAACACCTCCCAAAAGCTGACAGGGGCTTCTTCCGGCTG |
| | TTCTTCACTGGTATCTTTCATTCTCATGTTGAAAGCTTCCTTC |
| | AACTGTTGGGTCGTTGGCATGACCTCCTGCACCTCAAATTCC |
| | TTGAAAATATTCTGGATTTCGGCATAGTATTTCAGCAAGTCC |
| | GTATTGATTTCGGCTGCACTTTGCTTTAGCTTGTTGGTACAT |
| | CCGTTCTTTACCCGCTGCTTATCTGCATCCCATTTGGCTACG |
| | TCAATCCGGTAGCCCGTTGTAAACTCGATACGTTGGCTGGC |
| | AAAGATGACACGCATACGGATGGGTACGTTCTCTACGATTG |
| | GCACACCGTTCTTTTTCCGGCTCTCCAATGCAAAAATGATGT |
| | TGCGCTTGATATTCATAATTGGGTGCGTTTGAAATTCTACAC |
| | CCAAATATACACCCAATTATTGAGATAGCAAAAGACATTTA |
| | GAAACATTTACTTTTACTCTATATTGTAATTTACACTTGATT |
| | ATCAGTCGTTTGCAGTCTTATGATATTCTGTGAAAGTATAAG |
| | TTCGAGAGCCTGTCTCTCCGCAAAAAACGCTGAAAATCAGC |
| | AGATTGCAAAACAAACACCCTGTTTTACACCCAAGAATGTA |
| | AAGTCGGGTGTTTTTGTTTTATTTAAGATAATACAACCACTA |
| | CATAATAAAAGAGTAGCGATATTAAAAGAATCCGATGAGA |
| | AAAGACTAATATTTATCTATCCATTCAGTTTGATTTCTCAGG |
| | ACTTTACATCGTCCTGAAAGTATTTGTTGCAGTTCAACCTGT |
| | TGATAGTACGTACTAAGCTCTCATGTTTCACGTACTAAGCTC |
| | TCATGTTTAACGTACTAAGCTCTCATGTTTAACGAACTAAAC |
| | CCTCATGGCTAACGTACTAAGCTCTCATGGCTAACGTACTA |
| | AGCTCTCATGTTTCACGTACTAAGCTCTCATGTTTGAACAAT |
| | AAAATTAATATAAATCAGCAACTTAAATAGCCTCTAAGGTT |
| | TTAAGTTTTATAAGAAAAAAAAGAATATATAAGGCTTTTAA |
| | AGCTTTTAAGGTTTAACGGTTGTGGACAACAAGCCAGGGAT |
| | GTAACGCACTGAGAAGCCCTTAGAGCCTCTCAAAGCAATTT |
| | TGAGTGACACAGGAACACTTAACGGCTGACATGGGGCGGC |
| | CGCACGACGTACCGGACTCAGTAGGGAGAGCTGTATGTGGG |
| | TAG |
| SEQ ID NO: 18- | ATGTTAAATTTAAATTATGTAAAGATGAAAAAAATGAGATT |
| 04469-CL3- | TTTATTAGTATTATTAGTGGGTATATCTATTGTATCATGTAC |
| Nanoluciferase | TAATGACTTAGATGAAATGCTAGGGACTCAAAATTTGCAAA |
| nucleotide sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCACCGACAGCTTCTCCTCTGGGTTCCGGT |
| | GGCTCTGGTGGTGTTTTTACTCTGGAAGATTTTGTTGGCGAT |
| | TGGCGTCAGACCGCGGGTTATAATTTGGATCAAGTCCTGGA |
| | ACAGGGTGGCGTAAGCTCTCTGTTCCAGAACCTGGGTGTGA |
| | GCGTGACGCCGATTCAGCGCATCGTTCTGTCCGGCGAGAAC |
| | GGTCTGAAAATTGATATTCATGTGATCATCCCGTACGAAGG |
| | CCTGAGCGGTGACCAAATGGGTCAAATCGAGAAAATCTTTA |
| | AAGTCGTCTACCCAGTTGACGATCACCACTTCAAGGTTATCT |
| | TGCATTACGGTACGCTGGTGATTGATGGTGTGACCCCGAAT |
| | ATGATTGACTATTTCGGCCGTCCGTATGAAGGCATTGCCGTT |
| | TTTGACGGTAAAAAGATCACCGTCACCGGTACCCTGTGGAA |
| | TGGCAATAAGATTATTGACGAGCGTCTGATTAACCCGGACG |
| | GCAGCCTGCTGTTCCGCGTGACCATCAACGGTGTCACGGGT |
| | TGGCGTCTGTGCGAGCGCATCCTGGCATAA |
| SEQ ID NO: 19- | MLNLNYVKMKKMRFLLVLLVGISIVSCTNDLDEMLGTQNLQT |
| 04469-CL3- | RSVEPRFTGNISESQAIALMNKFLDAPSFFYFEDVIGFDPTTSIST |
| Nanoluciferase amino | GSFTVNRNGKTIHVRSIQVGVIGKNGKYGVYGTPVTSKEFYSL |
| acid sequence | RNGMVLHGEPIYEENDVERANPVGMRYYYTTNDVNHSQNCW |
| | IAINWADASTFESIIGWGGSGGGSPTASPLGSGGSGGVFTLEDF |
| | VGDWRQTAGYNLDQVLEQGGVSSLFQNLGVSVTPIQRIVLSG |
| | ENGLKIDIHVIIPYEGLSGDQMGQIEKIFKVVYPVDDHHFKVIL |
| | HYGTLVIDGVTPNMIDYFGRPYEGIAVFDGKKITVTGTLWNGN |
| | KIIDERLINPDGSLLFRVTINGVTGWRLCERILA |
| SEQ ID NO: 20- | GGSGG |
| GGSGG linker | |
| SEQ ID NO: 21- | GGGGS |
| (GGGGS)n linker | |
| SEQ ID NO: 22- | ATGTTAAATTTAAATTATGTAAAGATGAAAAAAATGAGATT |
| 04469-3XFLAG- | TTTATTAGTATTATTAGTGGGTATATCTATTGTATCATGTAC |
| 6XHis nucleotide | TAATGACTTAGATGAAATGCTAGGGACTCAAAATTTGCAAA |
| sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAATGG |
| | ACTACAAGGATCATGATGGGGATTACAAGGACCATGACATA |
| | GATTACAAAGATGATGATGATAAAGGGTCTAGTGATTACAA |
| | GGACGATGATGATAAGGACTACAAAGATGATGATGACAAA |
| | GATTATAAGGACGATGATGATAAAGGTCATCATCATCATCA |
| | TCACTAA |
| SEQ ID NO: 23- | MLNLNYVKMKKMRFLLVLLVGISIVSCTNDLDEMLGTQNLQT |
| 04469-3XFLAG- | RSVEPRFTGNISESQAIALMNKFLDAPSFFYFEDVIGFDPTTSIST |
| 6XHis amino acid | GSFTVNRNGKTIHVRSIQVGVIGKNGKYGVYGTPVTSKEFYSL |
| sequence | RNGMVLHGEPIYEENDVERANPVGMRYYYTTNDVNHSQNCW |
| | IAINWADASTFESIIGWGMDYKDHDGDYKDHDIDYKDDDDKG |
| | SSDYKDDDDKDYKDDDDKDYKDDDDKGHHHHHH |
| SEQ ID NO: 24- | ATGTTAAATTTAAATTATGTAAAGATGAAAAAAATGAGATT |
| 04469-3XFLAG-Fh8 | TTTATTAGTATTATTAGTGGGTATATCTATTGTATCATGTAC |
| domain nucleotide | TAATGACTTAGATGAAATGCTAGGGACTCAAAATTTGCAAA |
| sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAATGG |
| | ACTACAAGGATCATGATGGGGATTACAAGGACCATGACATA |
| | GATTACAAAGATGATGATGATAAAGGGTCACCATCGGTCCA |
| | AGAAGTTGAGAAGCTGCTTCATGTTCTTGACAGAAATGGTG |
| | ATGGAAAGGTGAGTGCAGAAGAACTGAAAGCATTCGCAGA |
| | TGACTCCAAATGTCCTTTGGACAGTAACAAAATCAAAGCGT |
| | TCATTAAGGAACATGACAAGAACAAGGACGGAAAACTGGA |
| | TCTGAAGGAGCTGGTTAGTATCCTTAGTTCTTAA |
| SEQ ID NO: 25- | MLNLNYVKMKKMRFLLVLLVGISIVSCTNDLDEMLGTQNLQT |
| 04469-3XFLAG-Fh8 | RSVEPRFTGNISESQAIALMNKFLDAPSFFYFEDVIGFDPTTSIST |
| domain amino acid | GSFTVNRNGKTIHVRSIQVGVIGKNGKYGVYGTPVTSKEFYSL |
| sequence | RNGMVLHGEPIYEENDVERANPVGMRYYYTTNDVNHSQNCW |
| | IAINWADASTFESIIGWGMDYKDHDGDYKDHDIDYKDDDDKG |
| | SPSVQEVEKLLHVLDRNGDGKVSAEELKAFADDSKCPLDSNKI |
| | KAFIKEHDKNKDGKLDLKELVSILSS |
| SEQ ID NO: 26- | ATGTTAAATTTAAATTATGTAAAGATGAAAAAAATGAGATT |
| 04469-3XFLAG- | TTTATTAGTATTATTAGTGGGTATATCTATTGTATCATGTAC |
| IL1Ra(Mm) nucleotide | TAATGACTTAGATGAAATGCTAGGGACTCAAAATTTGCAAA |
| sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAATGG |
| | ACTACAAGGATCATGATGGGGATTACAAGGACCATGACATA |
| | GATTACAAAGATGATGATGATAAAGGGTCACGGCCATCTGG |
| | AAAACGTCCTTGTAAGATGCAGGCATTTCGTATTTGGGATA |
| | CGAACCAAAAGACATTCTACCTTCGTAACAACCAATTGATC |
| | GCTGGTTATCTGCAAGGTCCTAACATCAAATTGGAGGAAAA |
| | AATCGACATGGTGCCTATTGATTTGCACAGCGTATTCCTTGG |
| | CATACATGGTGGCAAACTGTGTTTATCATGCGCAAAGTCGG |
| | GAGATGATATCAAACTTCAGCTGGAAGAAGTAAATATAACA |
| | GACCTGAGCAAAAACAAGGAAGAAGATAAACGCTTTACCT |
| | TTATACGTTCAGAAAAAGGACCTACCACATCTTTCGAGTCA |
| | GCTGCATGTCCGGGATGGTTTCTTTGCACAACTTTGGAGGCT |
| | GATCGCCCGGTGTCTTTAACAAACACGCCCGAAGAGCCACT |
| | TATCGTAACGAAATTTTACTTCCAGGAAGACCAAGGTTCCT |
| | AA |
| SEQ ID NO: 27- | MLNLNYVKMKKMRFLLVLLVGISIVSCTNDLDEMLGTQNLQT |
| 04469-3XFLAG- | RSVEPRFTGNISESQAIALMNKFLDAPSFFYFEDVIGFDPTTSIST |
| IL1Ra(Mm) amino acid | GSFTVNRNGKTIHVRSIQVGVIGKNGKYGVYGTPVTSKEFYSL |
| sequence | RNGMVLHGEPIYEENDVERANPVGMRYYYTTNDVNHSQNCW |
| | IAINWADASTFESIIGWGMDYKDHDGDYKDHDIDYKDDDDKG |
| | SRPSGKRPCKMQAFRIWDTNQKTFYLRNNQLIAGYLQGPNIKL |
| | EEKIDMVPIDLHSVFLGIHGGKLCLSCAKSGDDIKLQLEEVNIT |
| | DLSKNKEEDKRFTFIRSEKGPTTSFESAACPGWFLCTTLEADRP |
| | VSLTNTPEEPLIVTKFYFQEDQGS |
| SEQ ID NO: 28- | ATGTTAAATTTAAATTATGTAAAGATGAAAAAAATGAGATT |
| 04469-3XFLAG- | TTTATTAGTATTATTAGTGGGTATATCTATTGTATCATGTAC |
| Affibody1(zTNFa:185) | TAATGACTTAGATGAAATGCTAGGGACTCAAAATTTGCAAA |
| nucleotide sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCTGGAGGAGTGGATAATAAGTTCAATAA |
| | AGAATTAGGTTGGGCAATTGGAGAAATTGGCACCCTGCCAA |
| | ACCTGAATCATCAGCAATTTAGAGCCTTTATCCTGAGTTTAT |
| | GGGATGATCCTAGTCAGAGTGCAAATTTGCTTGCAGAGGCC |
| | AAAAAACTGAATGATGCCCAAGCCCCTAAAGGTTCCGACTA |
| | CAAGGATCATGATGGGGATTACAAGGACCATGACATAGATT |
| | ACAAAGATGATGATGATAAATAA |
| SEQ ID NO: 29- | MLNLNYVKMKKMRFLLVLLVGISIVSCTNDLDEMLGTQNLQT |
| 04469-3XFLAG- | RSVEPRFTGNISESQAIALMNKFLDAPSFFYFEDVIGFDPTTSIST |
| Affibody1(zTNFa:185) | GSFTVNRNGKTIHVRSIQVGVIGKNGKYGVYGTPVTSKEFYSL |
| amino acid sequence | RNGMVLHGEPIYEENDVERANPVGMRYYYTTNDVNHSQNCW |
| | IAINWADASTFESIIGWGGSGGGSGGVDNKENKELGWAIGEIG |
| | TLPNLNHQQFRAFILSLWDDPSQSANLLAEAKKLNDAQAPKGS |
| | DYKDHDGDYKDHDIDYKDDDDK |
| SEQ ID NO: 30- | ATGTTAAATTTAAATTATGTAAAGATGAAAAAAATGAGATT |
| 04469-3XFLAG-Trx | TTTATTAGTATTATTAGTGGGTATATCTATTGTATCATGTAC |
| nucleotide sequence | TAATGACTTAGATGAAATGCTAGGGACTCAAAATTTGCAAA |
| | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAATGG |
| | ACTACAAGGATCATGATGGGGATTACAAGGACCATGACATA |
| | GATTACAAAGATGATGATGATAAAGGGTCATCTGACAAAAT |
| | AATTCACCTTACGGATGACAGCTTTGATACGGACGTATTGA |
| | AGGCTGATGGAGCCATACTTGTGGATTTTTGGGCTGAGTGG |
| | TGTGGTCCGTGTAAAATGATTGCTCCAATATTGGATGAGAT |
| | TGCTGATGAATATCAAGGAAAACTTACTGTGGCCAAATTGA |
| | ATATTGATCAGAATCCAGGAACAGCCCCAAAATATGGCATT |
| | CGCGGTATTCCGACCTTGTTGCTGTTTAAAAATGGAGAGGT |
| | GGCTGCAACCAAAGTAGGAGCTTTATCTAAAGGCCAGTTGA |
| | AAGAATTCCTGGATGCAAATTTAGCTTAA |
| SEQ ID NO: 31- | MLNLNYVKMKKMRFLLVLLVGISIVSCTNDLDEMLGTQNLQT |
| 04469-3XFLAG-Trx | RSVEPRFTGNISESQAIALMNKFLDAPSFFYFEDVIGFDPTTSIST |
| amino acid sequence | GSFTVNRNGKTIHVRSIQVGVIGKNGKYGVYGTPVTSKEFYSL |
| | RNGMVLHGEPIYEENDVERANPVGMRYYYTTNDVNHSQNCW |
| | IAINWADASTFESIIGWGMDYKDHDGDYKDHDIDYKDDDDKG |
| | SSDKIIHLTDDSFDTDVLKADGAILVDFWAEWCGPCKMIAPIL |
| | DEIADEYQGKLTVAKLNIDQNPGTAPKYGIRGIPTLLLFKNGEV |
| | AATKVGALSKGQLKEFLDANLA |
| SEQ ID NO: 32- | ATGTTAAATTTAAATTATGTAAAGATGAAAAAAATGAGATT |
| 04469-3XFLAG- | TTTATTAGTATTATTAGTGGGTATATCTATTGTATCATGTAC |
| RNAseH nucleotide | TAATGACTTAGATGAAATGCTAGGGACTCAAAATTTGCAAA |
| sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAATGG |
| | ACTACAAGGATCATGATGGGGATTACAAGGACCATGACATA |
| | GATTACAAAGATGATGATGATAAAGGGTCACTGAAGCAAG |
| | TAGAAATATTTACAGATGGTTCCTGTCTGGGAAATCCTGGA |
| | CCGGGAGGTTATGGCGCTATATTACGCTATCGCGGCCGTGA |
| | AAAAACATTTTCCGCTGGTTATACGCGTACAACCAACAATC |
| | GCATGGAATTGATGGCTGCAATCGTGGCACTGGAGGCACTG |
| | AAAGAGCATTGTGAAGTCATCTTAAGCACGGACAGCCAATA |
| | CGTACGTCAGGGAATAACGCAATGGATCCACAATTGGAAG |
| | AAACGTGGCTGGAAGACGGCAGATAAAAAACCCGTGAAGA |
| | ACGTAGATTTATGGCAACGTTTAGATGCTGCTCTGGGGCAA |
| | CATCAAATCAAATGGGAATGGGTAAAAGGACATGCTGGAC |
| | ACCCGGAAAACGAAAGATGCGATGAGCTTGCGAGAGCTGC |
| | AGCAATGAACCCGACCTTGGAAGACACTGGATATCAGGTTG |
| | AGGTCGGTTCCTAA |
| SEQ ID NO: 33- | MLNLNYVKMKKMRFLLVLLVGISIVSCTNDLDEMLGTQNLQT |
| 04469-3XFLAG- | RSVEPRFTGNISESQAIALMNKFLDAPSFFYFEDVIGFDPTTSIST |
| RNAseH amino acid | GSFTVNRNGKTIHVRSIQVGVIGKNGKYGVYGTPVTSKEFYSL |
| sequence | RNGMVLHGEPIYEENDVERANPVGMRYYYTTNDVNHSQNCW |
| | IAINWADASTFESIIGWGMDYKDHDGDYKDHDIDYKDDDDKG |
| | SLKQVEIFTDGSCLGNPGPGGYGAILRYRGREKTFSAGYTRTT |
| | NNRMELMAAIVALEALKEHCEVILSTDSQYVRQGITQWIHNW |
| | KKRGWKTADKKPVKNVDLWQRLDAALGQHQIKWEWVKGH |
| | AGHPENERCDELARAAAMNPTLEDTGYQVEVGS |
| SEQ ID NO: 34- | ATGTTAAATTTAAATTATGTAAAGATGAAAAAAATGAGATT |
| 04469-GGSGG- | TTTATTAGTATTATTAGTGGGTATATCTATTGTATCATGTAC |
| VHH1-3XFLAG | TAATGACTTAGATGAAATGCTAGGGACTCAAAATTTGCAAA |
| nucleotide sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCAGTCCAGTTAGTTGAGAGCGGCGGTGG |
| | ACTGGTGCAAGCAGGTGGCAGCTTGAGCCTGTCCTGTAGTG |
| | CCTCTGGTCGTTCTTTATCTAATTACTATATGGGCTGGTTCA |
| | GACAGGCTCCTGGGAAGGAGAGAGAGCTGTTAGGCAATAT |
| | TAGCTGGCGTGGTTACAACATTTATTACAAGGACAGTGTGA |
| | AAGGTCGCTTTACGATTTCGCGGGACGATGCTAAGAACACC |
| | ATTTACTTACAGATGAATAGACTGAAGCCTGAAGACACTGC |
| | AGTGTATTACTGTGCTGCCAGTATTTTGCCTTTGTCCGACGA |
| | TCCTGGCTGGAACACATATTGGGGTCAGGGTACCCAGGTCA |
| | CAGTGTCTGGTTCCGACTACAAGGATCATGATGGGGATTAC |
| | AAGGACCATGACATAGATTACAAAGATGATGATGATAAAT |
| | AA |
| SEQ ID NO: 35- | MLNLNYVKMKKMRFLLVLLVGISIVSCTNDLDEMLGTQNLQT |
| 04469-GGSGG- | RSVEPRFTGNISESQAIALMNKFLDAPSFFYFEDVIGFDPTTSIST |
| VHH1-3XFLAG | GSFTVNRNGKTIHVRSIQVGVIGKNGKYGVYGTPVTSKEFYSL |
| amino acid sequence | RNGMVLHGEPIYEENDVERANPVGMRYYYTTNDVNHSQNCW |
| | IAINWADASTFESIIGWGGSGGGSVQLVESGGGLVQAGGSLSL |
| | SCSASGRSLSNYYMGWFRQAPGKERELLGNISWRGYNIYYKD |
| | SVKGRFTISRDDAKNTIYLQMNRLKPEDTAVYYCAASILPLSD |
| | DPGWNTYWGQGTQVTVSGSDYKDHDGDYKDHDIDYKDDDD |
| | K |
| SEQ ID NO: 36- | ATGTTAAATTTAAATTATGTAAAGATGAAAAAAATGAGATT |
| 04469-GGSGG-Fp- | TTTATTAGTATTATTAGTGGGTATATCTATTGTATCATGTAC |
| MAM3 peptide- | TAATGACTTAGATGAAATGCTAGGGACTCAAAATTTGCAAA |
| 3XFLAG nucleotide | CCCGTTCTGTTGAGCCTAGATTCACAGGAAATATATCAGAA |
| sequence | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCAGTAGGTAACACTTTCTTGCAGTCTACT |
| | ATCAACCGTACTATCGGTGTATTGGGTTCCGACTACAAGGA |
| | TCATGATGGGGATTACAAGGACCATGACATAGATTACAAAG |
| | ATGATGATGATAAATAA |
| SEQ ID NO: 37- | MLNLNYVKMKKMRFLLVLLVGISIVSCTNDLDEMLGTQNLQT |
| 04469-GGSGG-Fp- | RSVEPRFTGNISESQAIALMNKFLDAPSFFYFEDVIGFDPTTSIST |
| MAM3 peptide- | GSFTVNRNGKTIHVRSIQVGVIGKNGKYGVYGTPVTSKEFYSL |
| 3XFLAG amino acid | RNGMVLHGEPIYEENDVERANPVGMRYYYTTNDVNHSQNCW |
| sequence | IAINWADASTFESIIGWGGSGGGSVGNTFLQSTINRTIGVLGSD |
| | YKDHDGDYKDHDIDYKDDDDK |
| SEQ ID NO: 38- | ATGTTAAATTTAAATTATGTAAAGATGAAAAAAATGAGATT |
| 04469-GGSGG-MF-7- | TTTATTAGTATTATTAGTGGGTATATCTATTGTATCATGTAC |
| 3XFLAG nucleotide | TAATGACTTAGATGAAATGCTAGGGACTCAAAATTTGCAAA |
| sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCAATGTCTTACTCTGCTGGTTTCGGTTCCG |
| | ACTACAAGGATCATGATGGGGATTACAAGGACCATGACATA |
| | GATTACAAAGATGATGATGATAAATAA |
| SEQ ID NO: 39- | MLNLNYVKMKKMRFLLVLLVGISIVSCTNDLDEMLGTQNLQT |
| 04469-GGSGG-MF-7- | RSVEPRFTGNISESQAIALMNKFLDAPSFFYFEDVIGFDPTTSIST |
| 3XFLAG amino acid | GSFTVNRNGKTIHVRSIQVGVIGKNGKYGVYGTPVTSKEFYSL |
| sequence | RNGMVLHGEPIYEENDVERANPVGMRYYYTTNDVNHSQNCW |
| | IAINWADASTFESIIGWGGSGGGSMSYSAGFGSDYKDHDGDY |
| | KDHDIDYKDDDDK |
| SEQ ID NO: 40- | ATGTTAAATTTAAATTATGTAAAGATGAAAAAAATGAGATT |
| 04469-CL3-HS2Detox- | TTTATTAGTATTATTAGTGGGTATATCTATTGTATCATGTAC |
| 3XFLAG nucleotide | TAATGACTTAGATGAAATGCTAGGGACTCAAAATTTGCAAA |
| sequence | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCAGCTACAAAAAAATTACATTTCGAAACA |
| | TTACAACTTCACGTAGGACAGGAACAGCCAGACCCGGCTAC |
| | GGATGCTCGTGCAGTTCCTATTTATCAGACTACATCTTATGT |
| | ATTTCATAACTCGGCACATGCAGCCGCACGTTTTGGTTTACA |
| | AGATCCGGGTAATATTTATGGGCGTCTGACTAATTCCACAC |
| | AAGGTGTTTTTGAACAACGTGTGGCTGCATTGGAAGGGGGA |
| | GTGGCAGGACTTGCGGTAGCTTCCGGGGCTGCTGCCATTAC |
| | TTATGCGTTTCAGAACATTACTCGTGCAGGTGATCATGTTGT |
| | AGCTGCAAAGACTATTTACGGAGGTACTTATAACCTGCTGG |
| | CGCATACGTTGCCGACTTATGGTGTGACTGCTACATTTGTGG |
| | ACCCTGGTGATCTTTCTAATTTTGAGAAAGCTATTCAGGAG |
| | AATACAAAGGCGGTGTTTATCGAGACTTTGGGGAATCCTAA |
| | TTCGAATATTATAGATATAGAGGCTGTGGCGGAAATAGCAC |
| | ATCGCCATCACATACCTTTGATTATTGATAACACTTTTGGTA |
| | CTCCTTATCTGATTCGTCCCATCGAGCACGGAGCAGATATTG |
| | TGGTACATTCCGCTACAAAATTTATTGGTGGCCATGGAACTT |
| | CACTGGGTGGCGTGATTGTTGATTCAGGTAAATTTGACTGG |
| | GTGGCATCAGGTAAATTCCCTCAACTGACAGAATCGGATCC |
| | TTGCTATCATGGGGTGCGTTTTGTAGAGGCGGCAGGTCCGG |
| | CTGCTTATGCTGTCCGTATTCGTGCTATTTTATTGCGTGATA |
| | CCGGTGCAACTATCAGTCCGTTTAATGCTTTTATTCTGTTGC |
| | AGGGATTGGAGACACTTTCATTGCGTGTGGAACGTCATGTA |
| | GAGAATGCGTTGAAAGTGGTGGAGCATTTGAAAAATCATCC |
| | GAAGATCAAGAAAGTAAATCACCCTTCCTTGCCGGAACATC |
| | CTGACCATGCTCTGTATCAGCGTTATTTCCCCAATGGAGCTA |
| | GTTCTATTTTCACTATCGAAGTGAAAGGTGGTCAGGAAGAA |
| | GCACATCGTTTCATCGATAGTTTGGAAATCTTTTCTTTATTG |
| | GCCAATGTCGCCGATGTGAAGTCATTGGTTATCCATCCTGCC |
| | AGTACCACTCACTCCCAGTTGAATGCGGAAGAATTGGCGGA |
| | ACAGGGTATTTATCCGGGGACAGTGCGTTTATCAATAGGTA |
| | CGGAACATATAGATGATTTGCTGGCCGATTTGGATCAGGCA |
| | TTAGCTGGAATTGGTTCCGACTACAAGGATCATGATGGGGA |
| | TTACAAGGACCATGACATAGATTACAAAGATGATGATGATA |
| | AATAA |
| SEQ ID NO: 41- | MLNLNYVKMKKMRFLLVLLVGISIVSCTNDLDEMLGTQNLQT |
| 04469-CL3-HS2Detox- | RSVEPRFTGNISESQAIALMNKFLDAPSFFYFEDVIGFDPTTSIST |
| 3XFLAG amino acid | GSFTVNRNGKTIHVRSIQVGVIGKNGKYGVYGTPVTSKEFYSL |
| sequence | RNGMVLHGEPIYEENDVERANPVGMRYYYTTNDVNHSQNCW |
| | IAINWADASTFESIIGWGGSGGGSATKKLHFETLQLHVGQEQP |
| | DPATDARAVPIYQTTSYVFHNSAHAAARFGLQDPGNIYGRLTN |
| | STQGVFEQRVAALEGGVAGLAVASGAAAITYAFQNITRAGDH |
| | VVAAKTIYGGTYNLLAHTLPTYGVTATFVDPGDLSNFEKAIQE |
| | NTKAVFIETLGNPNSNIIDIEAVAEIAHRHHIPLIIDNTFGTPYLIR |
| | PIEHGADIVVHSATKFIGGHGTSLGGVIVDSGKFDWVASGKFP |
| | QLTESDPCYHGVRFVEAAGPAAYAVRIRAILLRDTGATISPFNA |
| | FILLQGLETLSLRVERHVENALKVVEHLKNHPKIKKVNHPSLPE |
| | HPDHALYQRYFPNGASSIFTIEVKGGQEEAHRFIDSLEIFSLLAN |
| | VADVKSLVIHPASTTHSQLNAEELAEQGIYPGTVRLSIGTEHID |
| | DLLADLDQALAGIGSDYKDHDGDYKDHDIDYKDDDDK |
| SEQ ID NO: 42- | GATAAAACGAAAGGCTCAGTCGAAAGACTGGGCCTTTCGTT |
| SZR1059; 4469- | TTACAATTGGGCTACCTTTTTTTTGTTTTGTTTGCAATGGTTA |
| GGSGG-NL! | ATCTATTGTTAAAATTTAAAGTTTCACTTGAACTTTCAAATA |
| nucleotide sequence | ATGTTCTTATATTTGCAGTGTCGAAAGAAACAAAGTAGCCT |
| | GGATCACACAACATTTAAAAAATAACATTATGAAAGCACTC |
| | GAAAAGAGATTCAAAGGTAATATTAACAATAATTTATTTTA |
| | ATGTTAAATTTAAATTATGTAAAGATGAAAAAAATGAGATT |
| | TTTATTAGTATTATTAGTGGGTATATCTATTGTATCATGTAC |
| | TAATGACTTAGATGAAATGCTAGGGACTCAAAATTTGCAAA |
| | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCTAGTGTTTTTACTCTGGAAGATTTTGTTG |
| | GCGATTGGCGTCAGACCGCGGGTTATAATTTGGATCAAGTC |
| | CTGGAACAGGGTGGCGTAAGCTCTCTGTTCCAGAACCTGGG |
| | TGTGAGCGTGACGCCGATTCAGCGCATCGTTCTGTCCGGCG |
| | AGAACGGTCTGAAAATTGATATTCATGTGATCATCCCGTAC |
| | GAAGGCCTGAGCGGTGACCAAATGGGTCAAATCGAGAAAA |
| | TCTTTAAAGTCGTCTACCCAGTTGACGATCACCACTTCAAGG |
| | TTATCTTGCATTACGGTACGCTGGTGATTGATGGTGTGACCC |
| | CGAATATGATTGACTATTTCGGCCGTCCGTATGAAGGCATT |
| | GCCGTTTTTGACGGTAAAAAGATCACCGTCACCGGTACCCT |
| | GTGGAATGGCAATAAGATTATTGACGAGCGTCTGATTAACC |
| | CGGACGGCAGCCTGCTGTTCCGCGTGACCATCAACGGTGTC |
| | ACGGGTTGGCGTCTGTGCGAGCGCATCCTGGCATAAGGTTC |
| | CTAAGGCCATCCTGACGGATGGCCTTTTTTTTGACTGCTATG |
| | ACTTGATACCGGCTATTACGAGCGCTTAAACGGCGCGCCTG |
| | ATAGGTGGGCTGCCCTTCCTGGTTGGCTTGGTTTCATCAGCC |
| | ATCCGCTTGCCCTCATCTGTTACGCCGGCGGTAGCCGGCCA |
| | GCCTCGCAGAGCAGGATTCCCGTTGAGCACCGCCAGGTGCG |
| | AATAAGGGACAGTGAAGAAGGAACACCCGCTCGCGGGTGG |
| | GCCTACTTCACCTATCCTGCCCGGCTGACGCCGTTGGATACA |
| | CCAAGGAAAGTCTACACGAACCCTTTGGCAAAATCCTGTAT |
| | ATCGTGCGAAAAAGGATGGATATACCGAAAAAATCGCTAT |
| | AATGACCCCGAAGCAGGGTTATGCAGCGGAAAAGCGGGAT |
| | TAAAAGTCGGGGATTGGTGAACAAAAAGGTGTTTCTCTCTT |
| | TAAGAGAAATATCGTTTTGCTAAACAGTTGATATTGAGGTA |
| | TCATTTTATCGTAAAAGACATTTTTGCTCAACAATTGCTTGA |
| | CGGAAATCAACAAATTTTAGCATTTTGTAAAAAAGTCGCTA |
| | TATAATTTGGTGAATTGGAGTTATTTTCATATTTTTGCATCC |
| | CGAAGAGTTTCTCTTAAAGAGAGAAACATCTTTTGCATACC |
| | TTTTCCGACCGAATTTTTATGTCGTAAAGAGGGGCTTTGCAG |
| | GGGGTGGACTCAGAAAGATGAGAATAGATGACTATTGTAGT |
| | TGAAACACATAGAAAGTTGCTGATATACAGACCGATACGCA |
| | TATCGGGATGAACCATGAGTACGTTCTTTTCTCAAAAAACA |
| | TAAATATTCGAAAAGAGATGCAATAAATTAAGGAGAGGTT |
| | ATAATGAACAAAGTAAATATAAAAGATAGTCAAAATTTTAT |
| | TACTTCAAAATATCACATAGAAAAAATAATGAATTGCATAA |
| | GTTTAGATGAAAAAGATAACATCTTTGAAATAGGTGCAGGG |
| | AAAGGTCATTTTACTGCTGGATTGGTAAAGAGATGTAATTT |
| | TGTAACGGCGATAGAAATTGATTCTAAATTATGTGAGGTAA |
| | CTCGTAATAAGCTCTTAAATTATCCTAACTATCAAATAGTAA |
| | ATGATGATATACTGAAATTTACATTTCCTAGCCACAATCCAT |
| | ATAAAATATTTGGCAGCATACCTTACAACATAAGCACAAAT |
| | ATAATTCGAAAAATTGTTTTTGAAAGTTCAGCCACAATAAG |
| | TTATTTAATAGTGGAATATGGTTTTGCTAAAATGTTATTAGA |
| | TACAAACAGATCACTAGCATTGCTGTTAATGGCAGAGGTAG |
| | ATATTTCTATATTAGCAAAAATTCCTAGGTATTATTTCCATC |
| | CAAAACCTAAAGTGGATAGCACATTAATTGTATTAAAAAGA |
| | AAGCCAGCAAAAATGGCATTTAAAGAGAGAAAAAAATATG |
| | AAACTTTTGTAATGAAATGGGTTAACAAAGAGTACGAAAAA |
| | CTGTTTACAAAAAATCAATTTAATAAAGCTTTAAAACATGC |
| | GAGAATATATGATATAAACAATATTAGTTTCGAACAATTTG |
| | TATCGCTATTTAATAGTTATAAAATATTTAACGGCTAAAAA |
| | CAATAGGCCACATGCAACTGTAAATGTTTACGCGGGTACCG |
| | ACACCGCGGTGGAGGGGAATTGTGTTACAACCAATTAACCA |
| | ATTCTGATTAGAAAAACTCATCGAGCATCAAATGAAACTGC |
| | AATTTATTCATATCAGGATTATCAATACCATATTTTTGAAAA |
| | AGCCGTTTCTGTAATGAAGGAGAAAACTCACCGAGGCAGTT |
| | CCATAGGATGGCAAGATCCTGGTATCGGTCTGCGATTCCGA |
| | CTCGTCCAACATCAATACAACCTATTAATTTCCCCTCGTCAA |
| | AAATAAGGTTATCAAGTGAGAAATCACCATGAGTGACGACT |
| | GAATCCGGTGAGAATGGCAAAAGCTTATGCATTTCTTTCCA |
| | GACTTGTTCAACAGGCCAGCCATTACGCTCGTCATCAAAAT |
| | CACTCGCATCAACCAAACCGTTATTCATTCGTGATTGCGCCT |
| | GAGCGAGGCGAAATACGCGATCGCTGTTAAAAGGACAATT |
| | ACAAACAGGAATCGAATGCAACCGGCGCAGGAACACTGCC |
| | AGCGCATCAACAATATTTTCACCTGAATCAGGATATTCTTCT |
| | AATACCTGGAATGCTGTTTTCCCGGGGATCGCAGTGGTGAG |
| | TAACCATGCATCATCAGGAGTACGGATAAAATGCTTGATGG |
| | TCGGAAGAGGCATAAATTCCGTCAGCCAGTTTAGTCTGACC |
| | ATCTCATCTGTAACATCATTGGCAACGCTACCTTTGCCATGT |
| | TTCAGAAACAACTCTGGCGCATCGGGCTTCCCATACAATCG |
| | ATAGATTGTCGCACCTGATTGCCCGACATTATCGCGAGCCC |
| | ATTTATACCCATATAAATCAGCATCCATGTTGGAATTTAATC |
| | GCGGCCTGGAGCAAGACGTTTCCCGTTGAATATGGCTCATA |
| | ACACCCCTTGTATTACTGTTTATGTAAGCAGACAGTTTTATT |
| | GTTCATGATGATATATTTTTATCTTGTGCAATGTAACATCAG |
| | AGATTTTGAGACACAACGTGGCTTTGTTGAATAAATCGAAC |
| | TTTTGCTGAGTTGAAGGATCAGGTCATTGGTAACTATCTATG |
| | AAACTGTTTGATACTTTTATAGTTGATTAAACTTGTTCATGG |
| | CATTTGCCTTAATATCATCCGCTATGTCAATGTAGGGTTTCA |
| | TAGCTTTGTAGTCGCTGTGTCCCGTCCATTTCATGACCACCT |
| | GTGCCGGGATTCCGAGAGCCAGCGCATTGCAGATGAATGTC |
| | CTTTTTCCTGCATGGGTACTGAGCAAAGCGTATTTGGGTGTG |
| | ACTTCATCAATACGTTCATTTCCCTTGTAGTAGGTTTCCCGT |
| | ACAGGCTCGTTGATTTCTGCCAGTTCGCCCAGCTCTTTCAGG |
| | TAATCGTTCATCTTCTGGTTGCTGATGACGGGCAGAGCCAT |
| | GTAATTCTCGAAATGGATGTCCTTGTATTTGTCCAGTATGGC |
| | TTTGCTGTATTTGTTCAGTTCAATCGTCAGGCTGTCGGCAGT |
| | CTTGACTGTGGTTATTTCGATGTGGTCGGACTTCACATCGCT |
| | TCTTTTCAGATTGCGAACATCCGAATACCGCAAACTCGTAA |
| | AGCAGCAGAACAGGAAAACATCACGCACACGTTCCAGGTA |
| | TTGCTTATCCTTGGGTATCTGGTAGTCTTTCAGCTTGTTCAG |
| | TTCATCCCAAGTCAGGAAGATTACTTTTTTCGAGGTGGTTTT |
| | CAGTTTCGGTTTGAACGTATCGTATGCAATGTTCTGATGATG |
| | TCCTTTCTTGAAGCTCCAGCGCAGGAACCATTIGAGGAATC |
| | CCATTTGCTTGCCGATGGTGCTGTTTCTCATATCCTTGGTGT |
| | CACGCAGGAAGTTGACGTATTCGTTCAATCCAAACTCGTTG |
| | AAATAGTTGAACGTTGCATCCTCCTTGAACTCTTTGAGGTGG |
| | TTCCTCACTGCTGCAAATTTTTCATAGGTGGATGCCGTCCAG |
| | TTATTCTGGTTACCGCACTCTTTTACAAACTCATCGAACACC |
| | TCCCAAAAGCTGACAGGGGCTTCTTCCGGCTGTTCTTCACTG |
| | GTATCTTTCATTCTCATGTTGAAAGCTTCCTTCAACTGTTGG |
| | GTCGTTGGCATGACCTCCTGCACCTCAAATTCCTTGAAAATA |
| | TTCTGGATTTCGGCATAGTATTTCAGCAAGTCCGTATTGATT |
| | TCGGCTGCACTTTGCTTTAGCTTGTTGGTACATCCGTTCTTT |
| | ACCCGCTGCTTATCTGCATCCCATTTGGCTACGTCAATCCGG |
| | TAGCCCGTTGTAAACTCGATACGTTGGCTGGCAAAGATGAC |
| | ACGCATACGGATGGGTACGTTCTCTACGATTGGCACACCGT |
| | TCTTTTTCCGGCTCTCCAATGCAAAAATGATGTTGCGCTTGA |
| | TATTCATAATTGGGTGCGTTTGAAATTCTACACCCAAATATA |
| | CACCCAATTATTGAGATAGCAAAAGACATTTAGAAACATTT |
| | ACTTTTACTCTATATTGTAATTTACACTTGATTATCAGTCGTT |
| | TGCAGTCTTATGATATTCTGTGAAAGTATAAGTTCGAGAGC |
| | CTGTCTCTCCGCAAAAAACGCTGAAAATCAGCAGATTGCAA |
| | AACAAACACCCTGTTTTACACCCAAGAATGTAAAGTCGGGT |
| | GTTTTTGTTTTATTTAAGATAATACAACCACTACATAATAAA |
| | AGAGTAGCGATATTAAAAGAATCCGATGAGAAAAGACTAA |
| | TATTTATCTATCCATTCAGTTTGATTTCTCAGGACTTTACATC |
| | GTCCTGAAAGTATTTGTTGCAGTTCAACCTGTTGATAGTACG |
| | TACTAAGCTCTCATGTTTCACGTACTAAGCTCTCATGTTTAA |
| | CGTACTAAGCTCTCATGTTTAACGAACTAAACCCTCATGGCT |
| | AACGTACTAAGCTCTCATGGCTAACGTACTAAGCTCTCATG |
| | TTTCACGTACTAAGCTCTCATGTTTGAACAATAAAATTAATA |
| | TAAATCAGCAACTTAAATAGCCTCTAAGGTTTTAAGTTTTAT |
| | AAGAAAAAAAAGAATATATAAGGCTTTTAAAGCTTTTAAGG |
| | TTTAACGGTTGTGGACAACAAGCCAGGGATGTAACGCACTG |
| | AGAAGCCCTTAGAGCCTCTCAAAGCAATTTTGAGTGACACA |
| | GGAACACTTAACGGCTGACATGGGGCGGCCGCACGACGTA |
| | CCGGACTCAGTAGGGAGAGCTGTATGTGGGTAG |
| SEQ ID NO: 43- | GATAAAACGAAAGGCTCAGTCGAAAGACTGGGCCTTTCGTT |
| sZR1060; 1E6-4469- | TTACAATTGGGCTACCTTTTTTTTGTTTTGTTTGCAATGGTTA |
| GGSGG-RAT-NL- | ATCTATTGTTAAAATTTAAAGTTTCACTTGAACTTTCAAATA |
| (GS)4! nucleotide | ATGTTCTTATATTTGCAGTGTCGAAAGAAACAAAGTAGCCT |
| sequence | GGATCACACAACATTTAAAAAATAACATTATGAAAGCACTC |
| | GAAAAGAGATTCAAAGGTAATATTAACAATAATTTATTTTA |
| | ATGTTAAATTTAAATTATGTAAAGATGAAAAAAATGAGATT |
| | TTTATTAGTATTATTAGTGGGTATATCTATTGTATCATGTAC |
| | TAATGACTTAGATGAAATGCTAGGGACTCAAAATTTGCAAA |
| | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGGGGAGGAT |
| | CCGGCGGAGGGTCACGTGCGACGGTTTTTACTCTGGAAGAT |
| | TTTGTTGGCGATTGGCGTCAGACCGCGGGTTATAATTTGGAT |
| | CAAGTCCTGGAACAGGGTGGCGTAAGCTCTCTGTTCCAGAA |
| | CCTGGGTGTGAGCGTGACGCCGATTCAGCGCATCGTTCTGT |
| | CCGGCGAGAACGGTCTGAAAATTGATATTCATGTGATCATC |
| | CCGTACGAAGGCCTGAGCGGTGACCAAATGGGTCAAATCG |
| | AGAAAATCTTTAAAGTCGTCTACCCAGTTGACGATCACCAC |
| | TTCAAGGTTATCTTGCATTACGGTACGCTGGTGATTGATGGT |
| | GTGACCCCGAATATGATTGACTATTTCGGCCGTCCGTATGA |
| | AGGCATTGCCGTTTTTGACGGTAAAAAGATCACCGTCACCG |
| | GTACCCTGTGGAATGGCAATAAGATTATTGACGAGCGTCTG |
| | ATTAACCCGGACGGCAGCCTGCTGTTCCGCGTGACCATCAA |
| | CGGTGTCACGGGTTGGCGTCTGTGCGAGCGCATCCTGGCAG |
| | GTTCCGGGTCTGGATCAGGTAGCTAAGGTTCCTAAGGCCAT |
| | CCTGACGGATGGCCTTTTTTTTGACTGCTATGACTTGATACC |
| | GGCTATTACGAGCGCTTAAACGGCGCGCCTGATAGGTGGGC |
| | TGCCCTTCCTGGTTGGCTTGGTTTCATCAGCCATCCGCTTGC |
| | CCTCATCTGTTACGCCGGCGGTAGCCGGCCAGCCTCGCAGA |
| | GCAGGATTCCCGTTGAGCACCGCCAGGTGCGAATAAGGGAC |
| | AGTGAAGAAGGAACACCCGCTCGCGGGGGGCCTACTTCAC |
| | CTATCCTGCCCGGCTGACGCCGTTGGATACACCAAGGAAAG |
| | TCTACACGAACCCTTTGGCAAAATCCTGTATATCGTGCGAA |
| | AAAGGATGGATATACCGAAAAAATCGCTATAATGACCCCG |
| | AAGCAGGGTTATGCAGCGGAAAAGCGGGATTAAAAGTCGG |
| | GGATTGGTGAACAAAAAGGTGTTTCTCTCTTTAAGAGAAAT |
| | ATCGTTTTGCTAAACAGTTGATATTGAGGTATCATTTTATCG |
| | TAAAAGACATTTTTGCTCAACAATTGCTTGACGGAAATCAA |
| | CAAATTTTAGCATTTTGTAAAAAAGTCGCTATATAATTTGGT |
| | GAATTGGAGTTATTTTCATATTTTTGCATCCCGAAGAGTTTC |
| | TCTTAAAGAGAGAAACATCTTTTGCATACCTTTTCCGACCGA |
| | ATTTTTATGTCGTAAAGAGGGGCTTTGCAGGGGGTGGACTC |
| | AGAAAGATGAGAATAGATGACTATTGTAGTTGAAACACATA |
| | GAAAGTTGCTGATATACAGACCGATACGCATATCGGGATGA |
| | ACCATGAGTACGTTCTTTTCTCAAAAAACATAAATATTCGA |
| | AAAGAGATGCAATAAATTAAGGAGAGGTTATAATGAACAA |
| | AGTAAATATAAAAGATAGTCAAAATTTTATTACTTCAAAAT |
| | ATCACATAGAAAAAATAATGAATTGCATAAGTTTAGATGAA |
| | AAAGATAACATCTTTGAAATAGGTGCAGGGAAAGGTCATTT |
| | TACTGCTGGATTGGTAAAGAGATGTAATTTTGTAACGGCGA |
| | TAGAAATTGATTCTAAATTATGTGAGGTAACTCGTAATAAG |
| | CTCTTAAATTATCCTAACTATCAAATAGTAAATGATGATATA |
| | CTGAAATTTACATTTCCTAGCCACAATCCATATAAAATATTT |
| | GGCAGCATACCTTACAACATAAGCACAAATATAATTCGAAA |
| | AATTGTTTTTGAAAGTTCAGCCACAATAAGTTATTTAATAGT |
| | GGAATATGGTTTTGCTAAAATGTTATTAGATACAAACAGAT |
| | CACTAGCATTGCTGTTAATGGCAGAGGTAGATATTTCTATAT |
| | TAGCAAAAATTCCTAGGTATTATTTCCATCCAAAACCTAAA |
| | GTGGATAGCACATTAATTGTATTAAAAAGAAAGCCAGCAAA |
| | AATGGCATTTAAAGAGAGAAAAAAATATGAAACTTTTGTAA |
| | TGAAATGGGTTAACAAAGAGTACGAAAAACTGTTTACAAA |
| | AAATCAATTTAATAAAGCTTTAAAACATGCGAGAATATATG |
| | ATATAAACAATATTAGTTTCGAACAATTTGTATCGCTATTTA |
| | ATAGTTATAAAATATTTAACGGCTAAAAACAATAGGCCACA |
| | TGCAACTGTAAATGTTTACGCGGGTACCGACACCGCGGTGG |
| | AGGGGAATTGTGTTACAACCAATTAACCAATTCTGATTAGA |
| | AAAACTCATCGAGCATCAAATGAAACTGCAATTTATTCATA |
| | TCAGGATTATCAATACCATATTTTTGAAAAAGCCGTTTCTGT |
| | AATGAAGGAGAAAACTCACCGAGGCAGTTCCATAGGATGG |
| | CAAGATCCTGGTATCGGTCTGCGATTCCGACTCGTCCAACA |
| | TCAATACAACCTATTAATTTCCCCTCGTCAAAAATAAGGTTA |
| | TCAAGTGAGAAATCACCATGAGTGACGACTGAATCCGGTGA |
| | GAATGGCAAAAGCTTATGCATTTCTTTCCAGACTTGTTCAAC |
| | AGGCCAGCCATTACGCTCGTCATCAAAATCACTCGCATCAA |
| | CCAAACCGTTATTCATTCGTGATTGCGCCTGAGCGAGGCGA |
| | AATACGCGATCGCTGTTAAAAGGACAATTACAAACAGGAAT |
| | CGAATGCAACCGGCGCAGGAACACTGCCAGCGCATCAACA |
| | ATATTTTCACCTGAATCAGGATATTCTTCTAATACCTGGAAT |
| | GCTGTTTTCCCGGGGATCGCAGTGGTGAGTAACCATGCATC |
| | ATCAGGAGTACGGATAAAATGCTTGATGGTCGGAAGAGGC |
| | ATAAATTCCGTCAGCCAGTTTAGTCTGACCATCTCATCTGTA |
| | ACATCATTGGCAACGCTACCTTTGCCATGTTTCAGAAACAA |
| | CTCTGGCGCATCGGGCTTCCCATACAATCGATAGATTGTCG |
| | CACCTGATTGCCCGACATTATCGCGAGCCCATTTATACCCAT |
| | ATAAATCAGCATCCATGTTGGAATTTAATCGCGGCCTGGAG |
| | CAAGACGTTTCCCGTTGAATATGGCTCATAACACCCCTTGTA |
| | TTACTGTTTATGTAAGCAGACAGTTTTATTGTTCATGATGAT |
| | ATATTTTTATCTTGTGCAATGTAACATCAGAGATTTTGAGAC |
| | ACAACGTGGCTTTGTTGAATAAATCGAACTTTTGCTGAGTTG |
| | AAGGATCAGGTCATTGGTAACTATCTATGAAACTGTTTGAT |
| | ACTTTTATAGTTGATTAAACTTGTTCATGGCATTTGCCTTAA |
| | TATCATCCGCTATGTCAATGTAGGGTTTCATAGCTTTGTAGT |
| | CGCTGTGTCCCGTCCATTTCATGACCACCTGTGCCGGGATTC |
| | CGAGAGCCAGCGCATTGCAGATGAATGTCCTTTTTCCTGCA |
| | TGGGTACTGAGCAAAGCGTATTTGGGTGTGACTTCATCAAT |
| | ACGTTCATTTCCCTTGTAGTAGGTTTCCCGTACAGGCTCGTT |
| | GATTTCTGCCAGTTCGCCCAGCTCTTTCAGGTAATCGTTCAT |
| | CTTCTGGTTGCTGATGACGGGCAGAGCCATGTAATTCTCGA |
| | AATGGATGTCCTTGTATTTGTCCAGTATGGCTTTGCTGTATT |
| | TGTTCAGTTCAATCGTCAGGCTGTCGGCAGTCTTGACTGTGG |
| | TTATTTCGATGTGGTCGGACTTCACATCGCTTCTTTTCAGAT |
| | TGCGAACATCCGAATACCGCAAACTCGTAAAGCAGCAGAA |
| | CAGGAAAACATCACGCACACGTTCCAGGTATTGCTTATCCT |
| | TGGGTATCTGGTAGTCTTTCAGCTTGTTCAGTTCATCCCAAG |
| | TCAGGAAGATTACTTTTTTCGAGGTGGTTTTCAGTTTCGGTT |
| | TGAACGTATCGTATGCAATGTTCTGATGATGTCCTTTCTTGA |
| | AGCTCCAGCGCAGGAACCATTTGAGGAATCCCATTTGCTTG |
| | CCGATGGTGCTGTTTCTCATATCCTTGGTGTCACGCAGGAAG |
| | TTGACGTATTCGTTCAATCCAAACTCGTTGAAATAGTTGAAC |
| | GTTGCATCCTCCTTGAACTCTTTGAGGTGGTTCCTCACTGCT |
| | GCAAATTTTTCATAGGTGGATGCCGTCCAGTTATTCTGGTTA |
| | CCGCACTCTTTTACAAACTCATCGAACACCTCCCAAAAGCT |
| | GACAGGGGCTTCTTCCGGCTGTTCTTCACTGGTATCTTTCAT |
| | TCTCATGTTGAAAGCTTCCTTCAACTGTTGGGTCGTTGGCAT |
| | GACCTCCTGCACCTCAAATTCCTTGAAAATATTCTGGATTTC |
| | GGCATAGTATTTCAGCAAGTCCGTATTGATTTCGGCTGCACT |
| | TTGCTTTAGCTTGTTGGTACATCCGTTCTTTACCCGCTGCTTA |
| | TCTGCATCCCATTTGGCTACGTCAATCCGGTAGCCCGTTGTA |
| | AACTCGATACGTTGGCTGGCAAAGATGACACGCATACGGAT |
| | GGGTACGTTCTCTACGATTGGCACACCGTTCTTTTTCCGGCT |
| | CTCCAATGCAAAAATGATGTTGCGCTTGATATTCATAATTG |
| | GGTGCGTTTGAAATTCTACACCCAAATATACACCCAATTATT |
| | GAGATAGCAAAAGACATTTAGAAACATTTACTTTTACTCTA |
| | TATTGTAATTTACACTTGATTATCAGTCGTTTGCAGTCTTAT |
| | GATATTCTGTGAAAGTATAAGTTCGAGAGCCTGTCTCTCCG |
| | CAAAAAACGCTGAAAATCAGCAGATTGCAAAACAAACACC |
| | CTGTTTTACACCCAAGAATGTAAAGTCGGGTGTTTTTGTTTT |
| | ATTTAAGATAATACAACCACTACATAATAAAAGAGTAGCGA |
| | TATTAAAAGAATCCGATGAGAAAAGACTAATATTTATCTAT |
| | CCATTCAGTTTGATTTCTCAGGACTTTACATCGTCCTGAAAG |
| | TATTTGTTGCAGTTCAACCTGTTGATAGTACGTACTAAGCTC |
| | TCATGTTTCACGTACTAAGCTCTCATGTTTAACGTACTAAGC |
| | TCTCATGTTTAACGAACTAAACCCTCATGGCTAACGTACTA |
| | AGCTCTCATGGCTAACGTACTAAGCTCTCATGTTTCACGTAC |
| | TAAGCTCTCATGTTTGAACAATAAAATTAATATAAATCAGC |
| | AACTTAAATAGCCTCTAAGGTTTTAAGTTTTATAAGAAAAA |
| | AAAGAATATATAAGGCTTTTAAAGCTTTTAAGGTTTAACGG |
| | TTGTGGACAACAAGCCAGGGATGTAACGCACTGAGAAGCC |
| | CTTAGAGCCTCTCAAAGCAATTTTGAGTGACACAGGAACAC |
| | TTAACGG |
| SEQ ID NO: 44- | GATAAAACGAAAGGCTCAGTCGAAAGACTGGGCCTTTCGTT |
| sZR1061; 1E6-4469- | TTACAATTGGGCTACCTTTTTTTTGTTTTGTTTGCAATGGTTA |
| P5B10-GGSGG-RAT- | ATCTATTGTTAAAATTTAAAGTTTCACTTGAACTTTCAAATA |
| NL-(GS)4! nucleotide | ATGTTCTTATATTTGCAGTGTCGAAAGAAACAAAGTAGCCT |
| sequence | GCGATTATCTTTAAATTTTATAAAATGTTAAATTTAAATTAC |
| | GTTAAGATGAAGAAAATGAGATTCTTATTGGTTTTATTGGT |
| | GGGGATATCGATTGTCTCTTGTACTAATGACTTAGATGAAA |
| | TGCTAGGGACTCAAAATTTGCAAACCCGTTCTGTTGAGCCT |
| | AGATTCACAGGAAATATATCAGAAAGTCAGGCGATTGCATT |
| | AATGAATAAATTTCTGGATGCACCCTCTTTTTTTTATTTTGA |
| | AGATGTCATAGGCTTCGATCCGACAACATCTATTTCCACAG |
| | GAAGTTTCACTGTCAACAGAAATGGGAAAACTATACATGTA |
| | CGTTCTATTCAAGTTGGTGTAATAGGAAAGAACGGAAAATA |
| | TGGAGTTTATGGAACTCCAGTAACGAGCAAAGAATTCTATT |
| | CATTGAGAAATGGGATGGTTTTGCATGGAGAACCTATTTAT |
| | GAAGAGAATGATGTAGAAAGAGCCAACCCAGTAGGAATGC |
| | GTTATTACTATACAACTAATGATGTAAATCATAGTCAAAAT |
| | TGCTGGATTGCGATCAATTGGGCTGATGCTTCTACATTTGAA |
| | AGTATAATTGGTTGGGGAGGATCCGGCGGAGGGTCACGTGC |
| | GACGGTTTTTACTCTGGAAGATTTTGTTGGCGATTGGCGTCA |
| | GACCGCGGGTTATAATTTGGATCAAGTCCTGGAACAGGGTG |
| | GCGTAAGCTCTCTGTTCCAGAACCTGGGTGTGAGCGTGACG |
| | CCGATTCAGCGCATCGTTCTGTCCGGCGAGAACGGTCTGAA |
| | AATTGATATTCATGTGATCATCCCGTACGAAGGCCTGAGCG |
| | GTGACCAAATGGGTCAAATCGAGAAAATCTTTAAAGTCGTC |
| | TACCCAGTTGACGATCACCACTTCAAGGTTATCTTGCATTAC |
| | GGTACGCTGGTGATTGATGGTGTGACCCCGAATATGATTGA |
| | CTATTTCGGCCGTCCGTATGAAGGCATTGCCGTTTTTGACGG |
| | TAAAAAGATCACCGTCACCGGTACCCTGTGGAATGGCAATA |
| | AGATTATTGACGAGCGTCTGATTAACCCGGACGGCAGCCTG |
| | CTGTTCCGCGTGACCATCAACGGTGTCACGGGTTGGCGTCT |
| | GTGCGAGCGCATCCTGGCAGGTTCCGGGTCTGGATCAGGTA |
| | GCTAAGGTTCCTAAGGCCATCCTGACGGATGGCCTTTTTTTT |
| | GACTGCTATGACTTGATACCGGCTATTACGAGCGCTTAAAC |
| | GGCGCGCCTGATAGGTGGGCTGCCCTTCCTGGTTGGCTTGG |
| | TTTCATCAGCCATCCGCTTGCCCTCATCTGTTACGCCGGCGG |
| | TAGCCGGCCAGCCTCGCAGAGCAGGATTCCCGTTGAGCACC |
| | GCCAGGTGCGAATAAGGGACAGTGAAGAAGGAACACCCGC |
| | TCGCGGGTGGGCCTACTTCACCTATCCTGCCCGGCTGACGC |
| | CGTTGGATACACCAAGGAAAGTCTACACGAACCCTTTGGCA |
| | AAATCCTGTATATCGTGCGAAAAAGGATGGATATACCGAAA |
| | AAATCGCTATAATGACCCCGAAGCAGGGTTATGCAGCGGAA |
| | AAGCGGGATTAAAAGTCGGGGATTGGTGAACAAAAAGGTG |
| | TTTCTCTCTTTAAGAGAAATATCGTTTTGCTAAACAGTTGAT |
| | ATTGAGGTATCATTTTATCGTAAAAGACATTTTTGCTCAACA |
| | ATTGCTTGACGGAAATCAACAAATTTTAGCATTTTGTAAAA |
| | AAGTCGCTATATAATTTGGTGAATTGGAGTTATTTTCATATT |
| | TTTGCATCCCGAAGAGTTTCTCTTAAAGAGAGAAACATCTTT |
| | TGCATACCTTTTCCGACCGAATTTTTATGTCGTAAAGAGGGG |
| | CTTTGCAGGGGGTGGACTCAGAAAGATGAGAATAGATGACT |
| | ATTGTAGTTGAAACACATAGAAAGTTGCTGATATACAGACC |
| | GATACGCATATCGGGATGAACCATGAGTACGTTCTTTTCTC |
| | AAAAAACATAAATATTCGAAAAGAGATGCAATAAATTAAG |
| | GAGAGGTTATAATGAACAAAGTAAATATAAAAGATAGTCA |
| | AAATTTTATTACTTCAAAATATCACATAGAAAAAATAATGA |
| | ATTGCATAAGTTTAGATGAAAAAGATAACATCTTTGAAATA |
| | GGTGCAGGGAAAGGTCATTTTACTGCTGGATTGGTAAAGAG |
| | ATGTAATTTTGTAACGGCGATAGAAATTGATTCTAAATTAT |
| | GTGAGGTAACTCGTAATAAGCTCTTAAATTATCCTAACTATC |
| | AAATAGTAAATGATGATATACTGAAATTTACATTTCCTAGC |
| | CACAATCCATATAAAATATTTGGCAGCATACCTTACAACAT |
| | AAGCACAAATATAATTCGAAAAATTGTTTTTGAAAGTTCAG |
| | CCACAATAAGTTATTTAATAGTGGAATATGGTTTTGCTAAA |
| | ATGTTATTAGATACAAACAGATCACTAGCATTGCTGTTAAT |
| | GGCAGAGGTAGATATTTCTATATTAGCAAAAATTCCTAGGT |
| | ATTATTTCCATCCAAAACCTAAAGTGGATAGCACATTAATT |
| | GTATTAAAAAGAAAGCCAGCAAAAATGGCATTTAAAGAGA |
| | GAAAAAAATATGAAACTTTTGTAATGAAATGGGTTAACAAA |
| | GAGTACGAAAAACTGTTTACAAAAAATCAATTTAATAAAGC |
| | TTTAAAACATGCGAGAATATATGATATAAACAATATTAGTT |
| | TCGAACAATTTGTATCGCTATTTAATAGTTATAAAATATTTA |
| | ACGGCTAAAAACAATAGGCCACATGCAACTGTAAATGTTTA |
| | CGCGGGTACCGACACCGCGGTGGAGGGGAATTGTGTTACAA |
| | CCAATTAACCAATTCTGATTAGAAAAACTCATCGAGCATCA |
| | AATGAAACTGCAATTTATTCATATCAGGATTATCAATACCA |
| | TATTTTTGAAAAAGCCGTTTCTGTAATGAAGGAGAAAACTC |
| | ACCGAGGCAGTTCCATAGGATGGCAAGATCCTGGTATCGGT |
| | CTGCGATTCCGACTCGTCCAACATCAATACAACCTATTAATT |
| | TCCCCTCGTCAAAAATAAGGTTATCAAGTGAGAAATCACCA |
| | TGAGTGACGACTGAATCCGGTGAGAATGGCAAAAGCTTATG |
| | CATTTCTTTCCAGACTTGTTCAACAGGCCAGCCATTACGCTC |
| | GTCATCAAAATCACTCGCATCAACCAAACCGTTATTCATTC |
| | GTGATTGCGCCTGAGCGAGGCGAAATACGCGATCGCTGTTA |
| | AAAGGACAATTACAAACAGGAATCGAATGCAACCGGCGCA |
| | GGAACACTGCCAGCGCATCAACAATATTTTCACCTGAATCA |
| | GGATATTCTTCTAATACCTGGAATGCTGTTTTCCCGGGGATC |
| | GCAGTGGTGAGTAACCATGCATCATCAGGAGTACGGATAAA |
| | ATGCTTGATGGTCGGAAGAGGCATAAATTCCGTCAGCCAGT |
| | TTAGTCTGACCATCTCATCTGTAACATCATTGGCAACGCTAC |
| | CTTTGCCATGTTTCAGAAACAACTCTGGCGCATCGGGCTTCC |
| | CATACAATCGATAGATTGTCGCACCTGATTGCCCGACATTA |
| | TCGCGAGCCCATTTATACCCATATAAATCAGCATCCATGTTG |
| | GAATTTAATCGCGGCCTGGAGCAAGACGTTTCCCGTTGAAT |
| | ATGGCTCATAACACCCCTTGTATTACTGTTTATGTAAGCAGA |
| | CAGTTTTATTGTTCATGATGATATATTTTTATCTTGTGCAAT |
| | GTAACATCAGAGATTTTGAGACACAACGTGGCTTTGTTGAA |
| | TAAATCGAACTTTTGCTGAGTTGAAGGATCAGGTCATTGGT |
| | AACTATCTATGAAACTGTTTGATACTTTTATAGTTGATTAAA |
| | CTTGTTCATGGCATTTGCCTTAATATCATCCGCTATGTCAAT |
| | GTAGGGTTTCATAGCTTTGTAGTCGCTGTGTCCCGTCCATTT |
| | CATGACCACCTGTGCCGGGATTCCGAGAGCCAGCGCATTGC |
| | AGATGAATGTCCTTTTTCCTGCATGGGTACTGAGCAAAGCG |
| | TATTTGGGTGTGACTTCATCAATACGTTCATTTCCCTTGTAG |
| | TAGGTTTCCCGTACAGGCTCGTTGATTTCTGCCAGTTCGCCC |
| | AGCTCTTTCAGGTAATCGTTCATCTTCTGGTTGCTGATGACG |
| | GGCAGAGCCATGTAATTCTCGAAATGGATGTCCTTGTATTT |
| | GTCCAGTATGGCTTTGCTGTATTTGTTCAGTTCAATCGTCAG |
| | GCTGTCGGCAGTCTTGACTGTGGTTATTTCGATGTGGTCGGA |
| | CTTCACATCGCTTCTTTTCAGATTGCGAACATCCGAATACCG |
| | CAAACTCGTAAAGCAGCAGAACAGGAAAACATCACGCACA |
| | CGTTCCAGGTATTGCTTATCCTTGGGTATCTGGTAGTCTTTC |
| | AGCTTGTTCAGTTCATCCCAAGTCAGGAAGATTACTTTTTTC |
| | GAGGTGGTTTTCAGTTTCGGTTTGAACGTATCGTATGCAATG |
| | TTCTGATGATGTCCTTTCTTGAAGCTCCAGCGCAGGAACCAT |
| | TTGAGGAATCCCATTTGCTTGCCGATGGTGCTGTTTCTCATA |
| | TCCTTGGTGTCACGCAGGAAGTTGACGTATTCGTTCAATCCA |
| | AACTCGTTGAAATAGTTGAACGTTGCATCCTCCTTGAACTCT |
| | TTGAGGTGGTTCCTCACTGCTGCAAATTTTTCATAGGTGGAT |
| | GCCGTCCAGTTATTCTGGTTACCGCACTCTTTTACAAACTCA |
| | TCGAACACCTCCCAAAAGCTGACAGGGGCTTCTTCCGGCTG |
| | TTCTTCACTGGTATCTTTCATTCTCATGTTGAAAGCTTCCTTC |
| | AACTGTTGGGTCGTTGGCATGACCTCCTGCACCTCAAATTCC |
| | TTGAAAATATTCTGGATTTCGGCATAGTATTTCAGCAAGTCC |
| | GTATTGATTTCGGCTGCACTTTGCTTTAGCTTGTTGGTACAT |
| | CCGTTCTTTACCCGCTGCTTATCTGCATCCCATTTGGCTACG |
| | TCAATCCGGTAGCCCGTTGTAAACTCGATACGTTGGCTGGC |
| | AAAGATGACACGCATACGGATGGGTACGTTCTCTACGATTG |
| | GCACACCGTTCTTTTTCCGGCTCTCCAATGCAAAAATGATGT |
| | TGCGCTTGATATTCATAATTGGGTGCGTTTGAAATTCTACAC |
| | CCAAATATACACCCAATTATTGAGATAGCAAAAGACATTTA |
| | GAAACATTTACTTTTACTCTATATTGTAATTTACACTTGATT |
| | ATCAGTCGTTTGCAGTCTTATGATATTCTGTGAAAGTATAAG |
| | TTCGAGAGCCTGTCTCTCCGCAAAAAACGCTGAAAATCAGC |
| | AGATTGCAAAACAAACACCCTGTTTTACACCCAAGAATGTA |
| | AAGTCGGGTGTTTTTGTTTTATTTAAGATAATACAACCACTA |
| | CATAATAAAAGAGTAGCGATATTAAAAGAATCCGATGAGA |
| | AAAGACTAATATTTATCTATCCATTCAGTTTGATTTCTCAGG |
| | ACTTTACATCGTCCTGAAAGTATTTGTTGCAGTTCAACCTGT |
| | TGATAGTACGTACTAAGCTCTCATGTTTCACGTACTAAGCTC |
| | TCATGTTTAACGTACTAAGCTCTCATGTTTAACGAACTAAAC |
| | CCTCATGGCTAACGTACTAAGCTCTCATGGCTAACGTACTA |
| | AGCTCTCATGTTTCACGTACTAAGCTCTCATGTTTGAACAAT |
| | AAAATTAATATAAATCAGCAACTTAAATAGCCTCTAAGGTT |
| | TTAAGTTTTATAAGAAAAAAAAGAATATATAAGGCTTTTAA |
| | AGCTTTTAAGGTTTAACGGTTGTGGACAACAAGCCAGGGAT |
| | GTAACGCACTGAGAAGCCCTTAGAGCCTCTCAAAGCAATTT |
| | TGAGTGACACAGGAACACTTAACGGCTGACATGGGGCGGC |
| | CGCACGACGTACCGGACTCAGTAGGGAGAGCTGTATGTGGG |
| | TAG |
| SEQ ID NO: 45- | GATAAAACGAAAGGCTCAGTCGAAAGACTGGGCCTTTCGTT |
| sZR1062; 1E6-4469- | TTACAATTGGGCTACCTTTTTTTTGTTTTGTTTGCAATGGTTA |
| P5017-GGSGG-RAT- | ATCTATTGTTAAAATTTAAAGTTTCACTTGAACTTTCAAATA |
| NL-(GS)4! nucleotide | ATGTTCTTATATTTGCAGTGTCGAAAGAAACAAAGTAGCCT |
| sequence | GGATCTACTTTTAAAATTTATAAAATGTTGAATTTAAATTAT |
| | GTGAAAATGAAGAAAATGAGATTTCTATTAGTATTATTGGT |
| | CGGGATATCTATTGTATCATGTACTAATGACTTAGATGAAA |
| | TGCTAGGGACTCAAAATTTGCAAACCCGTTCTGTTGAGCCT |
| | AGATTCACAGGAAATATATCAGAAAGTCAGGCGATTGCATT |
| | AATGAATAAATTTCTGGATGCACCCTCTTTTTTTTATTTTGA |
| | AGATGTCATAGGCTTCGATCCGACAACATCTATTTCCACAG |
| | GAAGTTTCACTGTCAACAGAAATGGGAAAACTATACATGTA |
| | CGTTCTATTCAAGTTGGTGTAATAGGAAAGAACGGAAAATA |
| | TGGAGTTTATGGAACTCCAGTAACGAGCAAAGAATTCTATT |
| | CATTGAGAAATGGGATGGTTTTGCATGGAGAACCTATTTAT |
| | GAAGAGAATGATGTAGAAAGAGCCAACCCAGTAGGAATGC |
| | GTTATTACTATACAACTAATGATGTAAATCATAGTCAAAAT |
| | TGCTGGATTGCGATCAATTGGGCTGATGCTTCTACATTTGAA |
| | AGTATAATTGGTTGGGGAGGATCCGGCGGAGGGTCACGTGC |
| | GACGGTTTTTACTCTGGAAGATTTTGTTGGCGATTGGCGTCA |
| | GACCGCGGGTTATAATTTGGATCAAGTCCTGGAACAGGGTG |
| | GCGTAAGCTCTCTGTTCCAGAACCTGGGTGTGAGCGTGACG |
| | CCGATTCAGCGCATCGTTCTGTCCGGCGAGAACGGTCTGAA |
| | AATTGATATTCATGTGATCATCCCGTACGAAGGCCTGAGCG |
| | GTGACCAAATGGGTCAAATCGAGAAAATCTTTAAAGTCGTC |
| | TACCCAGTTGACGATCACCACTTCAAGGTTATCTTGCATTAC |
| | GGTACGCTGGTGATTGATGGTGTGACCCCGAATATGATTGA |
| | CTATTTCGGCCGTCCGTATGAAGGCATTGCCGTTTTTGACGG |
| | TAAAAAGATCACCGTCACCGGTACCCTGTGGAATGGCAATA |
| | AGATTATTGACGAGCGTCTGATTAACCCGGACGGCAGCCTG |
| | CTGTTCCGCGTGACCATCAACGGTGTCACGGGTTGGCGTCT |
| | GTGCGAGCGCATCCTGGCAGGTTCCGGGTCTGGATCAGGTA |
| | GCTAAGGTTCCTAAGGCCATCCTGACGGATGGCCTTTTTTTT |
| | GACTGCTATGACTTGATACCGGCTATTACGAGCGCTTAAAC |
| | GGCGCGCCTGATAGGTGGGCTGCCCTTCCTGGTTGGCTTGG |
| | TTTCATCAGCCATCCGCTTGCCCTCATCTGTTACGCCGGCGG |
| | TAGCCGGCCAGCCTCGCAGAGCAGGATTCCCGTTGAGCACC |
| | GCCAGGTGCGAATAAGGGACAGTGAAGAAGGAACACCCGC |
| | TCGCGGGTGGGCCTACTTCACCTATCCTGCCCGGCTGACGC |
| | CGTTGGATACACCAAGGAAAGTCTACACGAACCCTTTGGCA |
| | AAATCCTGTATATCGTGCGAAAAAGGATGGATATACCGAAA |
| | AAATCGCTATAATGACCCCGAAGCAGGGTTATGCAGCGGAA |
| | AAGCGGGATTAAAAGTCGGGGATTGGTGAACAAAAAGGTG |
| | TTTCTCTCTTTAAGAGAAATATCGTTTTGCTAAACAGTTGAT |
| | ATTGAGGTATCATTTTATCGTAAAAGACATTTTTGCTCAACA |
| | ATTGCTTGACGGAAATCAACAAATTTTAGCATTTTGTAAAA |
| | AAGTCGCTATATAATTTGGTGAATTGGAGTTATTTTCATATT |
| | TTTGCATCCCGAAGAGTTTCTCTTAAAGAGAGAAACATCTTT |
| | TGCATACCTTTTCCGACCGAATTTTTATGTCGTAAAGAGGGG |
| | CTTTGCAGGGGGTGGACTCAGAAAGATGAGAATAGATGACT |
| | ATTGTAGTTGAAACACATAGAAAGTTGCTGATATACAGACC |
| | GATACGCATATCGGGATGAACCATGAGTACGTTCTTTTCTC |
| | AAAAAACATAAATATTCGAAAAGAGATGCAATAAATTAAG |
| | GAGAGGTTATAATGAACAAAGTAAATATAAAAGATAGTCA |
| | AAATTTTATTACTTCAAAATATCACATAGAAAAAATAATGA |
| | ATTGCATAAGTTTAGATGAAAAAGATAACATCTTTGAAATA |
| | GGTGCAGGGAAAGGTCATTTTACTGCTGGATTGGTAAAGAG |
| | ATGTAATTTTGTAACGGCGATAGAAATTGATTCTAAATTAT |
| | GTGAGGTAACTCGTAATAAGCTCTTAAATTATCCTAACTATC |
| | AAATAGTAAATGATGATATACTGAAATTTACATTTCCTAGC |
| | CACAATCCATATAAAATATTTGGCAGCATACCTTACAACAT |
| | AAGCACAAATATAATTCGAAAAATTGTTTTTGAAAGTTCAG |
| | CCACAATAAGTTATTTAATAGTGGAATATGGTTTTGCTAAA |
| | ATGTTATTAGATACAAACAGATCACTAGCATTGCTGTTAAT |
| | GGCAGAGGTAGATATTTCTATATTAGCAAAAATTCCTAGGT |
| | ATTATTTCCATCCAAAACCTAAAGTGGATAGCACATTAATT |
| | GTATTAAAAAGAAAGCCAGCAAAAATGGCATTTAAAGAGA |
| | GAAAAAAATATGAAACTTTTGTAATGAAATGGGTTAACAAA |
| | GAGTACGAAAAACTGTTTACAAAAAATCAATTTAATAAAGC |
| | TTTAAAACATGCGAGAATATATGATATAAACAATATTAGTT |
| | TCGAACAATTTGTATCGCTATTTAATAGTTATAAAATATTTA |
| | ACGGCTAAAAACAATAGGCCACATGCAACTGTAAATGTTTA |
| | CGCGGGTACCGACACCGCGGTGGAGGGGAATTGTGTTACAA |
| | CCAATTAACCAATTCTGATTAGAAAAACTCATCGAGCATCA |
| | AATGAAACTGCAATTTATTCATATCAGGATTATCAATACCA |
| | TATTTTTGAAAAAGCCGTTTCTGTAATGAAGGAGAAAACTC |
| | ACCGAGGCAGTTCCATAGGATGGCAAGATCCTGGTATCGGT |
| | CTGCGATTCCGACTCGTCCAACATCAATACAACCTATTAATT |
| | TCCCCTCGTCAAAAATAAGGTTATCAAGTGAGAAATCACCA |
| | TGAGTGACGACTGAATCCGGTGAGAATGGCAAAAGCTTATG |
| | CATTTCTTTCCAGACTTGTTCAACAGGCCAGCCATTACGCTC |
| | GTCATCAAAATCACTCGCATCAACCAAACCGTTATTCATTC |
| | GTGATTGCGCCTGAGCGAGGCGAAATACGCGATCGCTGTTA |
| | AAAGGACAATTACAAACAGGAATCGAATGCAACCGGCGCA |
| | GGAACACTGCCAGCGCATCAACAATATTTTCACCTGAATCA |
| | GGATATTCTTCTAATACCTGGAATGCTGTTTTCCCGGGGATC |
| | GCAGTGGTGAGTAACCATGCATCATCAGGAGTACGGATAAA |
| | ATGCTTGATGGTCGGAAGAGGCATAAATTCCGTCAGCCAGT |
| | TTAGTCTGACCATCTCATCTGTAACATCATTGGCAACGCTAC |
| | CTTTGCCATGTTTCAGAAACAACTCTGGCGCATCGGGCTTCC |
| | CATACAATCGATAGATTGTCGCACCTGATTGCCCGACATTA |
| | TCGCGAGCCCATTTATACCCATATAAATCAGCATCCATGTTG |
| | GAATTTAATCGCGGCCTGGAGCAAGACGTTTCCCGTTGAAT |
| | ATGGCTCATAACACCCCTTGTATTACTGTTTATGTAAGCAGA |
| | CAGTTTTATTGTTCATGATGATATATTTTTATCTTGTGCAAT |
| | GTAACATCAGAGATTTTGAGACACAACGTGGCTTTGTTGAA |
| | TAAATCGAACTTTTGCTGAGTTGAAGGATCAGGTCATTGGT |
| | AACTATCTATGAAACTGTTTGATACTTTTATAGTTGATTAAA |
| | CTTGTTCATGGCATTTGCCTTAATATCATCCGCTATGTCAAT |
| | GTAGGGTTTCATAGCTTTGTAGTCGCTGTGTCCCGTCCATTT |
| | CATGACCACCTGTGCCGGGATTCCGAGAGCCAGCGCATTGC |
| | AGATGAATGTCCTTTTTCCTGCATGGGTACTGAGCAAAGCG |
| | TATTTGGGTGTGACTTCATCAATACGTTCATTTCCCTTGTAG |
| | TAGGTTTCCCGTACAGGCTCGTTGATTTCTGCCAGTTCGCCC |
| | AGCTCTTTCAGGTAATCGTTCATCTTCTGGTTGCTGATGACG |
| | GGCAGAGCCATGTAATTCTCGAAATGGATGTCCTTGTATTT |
| | GTCCAGTATGGCTTTGCTGTATTTGTTCAGTTCAATCGTCAG |
| | GCTGTCGGCAGTCTTGACTGTGGTTATTTCGATGTGGTCGGA |
| | CTTCACATCGCTTCTTTTCAGATTGCGAACATCCGAATACCG |
| | CAAACTCGTAAAGCAGCAGAACAGGAAAACATCACGCACA |
| | CGTTCCAGGTATTGCTTATCCTTGGGTATCTGGTAGTCTTTC |
| | AGCTTGTTCAGTTCATCCCAAGTCAGGAAGATTACTTTTTTC |
| | GAGGTGGTTTTCAGTTTCGGTTTGAACGTATCGTATGCAATG |
| | TTCTGATGATGTCCTTTCTTGAAGCTCCAGCGCAGGAACCAT |
| | TTGAGGAATCCCATTTGCTTGCCGATGGTGCTGTTTCTCATA |
| | TCCTTGGTGTCACGCAGGAAGTTGACGTATTCGTTCAATCCA |
| | AACTCGTTGAAATAGTTGAACGTTGCATCCTCCTTGAACTCT |
| | TTGAGGTGGTTCCTCACTGCTGCAAATTTTTCATAGGTGGAT |
| | GCCGTCCAGTTATTCTGGTTACCGCACTCTTTTACAAACTCA |
| | TCGAACACCTCCCAAAAGCTGACAGGGGCTTCTTCCGGCTG |
| | TTCTTCACTGGTATCTTTCATTCTCATGTTGAAAGCTTCCTTC |
| | AACTGTTGGGTCGTTGGCATGACCTCCTGCACCTCAAATTCC |
| | TTGAAAATATTCTGGATTTCGGCATAGTATTTCAGCAAGTCC |
| | GTATTGATTTCGGCTGCACTTTGCTTTAGCTTGTTGGTACAT |
| | CCGTTCTTTACCCGCTGCTTATCTGCATCCCATTTGGCTACG |
| | TCAATCCGGTAGCCCGTTGTAAACTCGATACGTTGGCTGGC |
| | AAAGATGACACGCATACGGATGGGTACGTTCTCTACGATTG |
| | GCACACCGTTCTTTTTCCGGCTCTCCAATGCAAAAATGATGT |
| | TGCGCTTGATATTCATAATTGGGTGCGTTTGAAATTCTACAC |
| | CCAAATATACACCCAATTATTGAGATAGCAAAAGACATTTA |
| | GAAACATTTACTTTTACTCTATATTGTAATTTACACTTGATT |
| | ATCAGTCGTTTGCAGTCTTATGATATTCTGTGAAAGTATAAG |
| | TTCGAGAGCCTGTCTCTCCGCAAAAAACGCTGAAAATCAGC |
| | AGATTGCAAAACAAACACCCTGTTTTACACCCAAGAATGTA |
| | AAGTCGGGTGTTTTTGTTTTATTTAAGATAATACAACCACTA |
| | CATAATAAAAGAGTAGCGATATTAAAAGAATCCGATGAGA |
| | AAAGACTAATATTTATCTATCCATTCAGTTTGATTTCTCAGG |
| | ACTTTACATCGTCCTGAAAGTATTTGTTGCAGTTCAACCTGT |
| | TGATAGTACGTACTAAGCTCTCATGTTTCACGTACTAAGCTC |
| | TCATGTTTAACGTACTAAGCTCTCATGTTTAACGAACTAAAC |
| | CCTCATGGCTAACGTACTAAGCTCTCATGGCTAACGTACTA |
| | AGCTCTCATGTTTCACGTACTAAGCTCTCATGTTTGAACAAT |
| | AAAATTAATATAAATCAGCAACTTAAATAGCCTCTAAGGTT |
| | TTAAGTTTTATAAGAAAAAAAAGAATATATAAGGCTTTTAA |
| | AGCTTTTAAGGTTTAACGGTTGTGGACAACAAGCCAGGGAT |
| | GTAACGCACTGAGAAGCCCTTAGAGCCTCTCAAAGCAATTT |
| | TGAGTGACACAGGAACACTTAACGGCTGACATGGGGCGGC |
| | CGCACGACGTACCGGACTCAGTAGGGAGAGCTGTATGTGGG |
| | TAG |
| SEQ ID NO: 46- | GATAAAACGAAAGGCTCAGTCGAAAGACTGGGCCTTTCGTT |
| SZR1063; 1E6-4469- | TTACAATTGGGCTACCTTTTTTTTGTTTTGTTTGCAATGGTTA |
| P5B10-GGSGG-RAT- | ATCTATTGTTAAAATTTAAAGTTTCACTTGAACTTTCAAATA |
| NL-PQP! nucleotide | ATGTTCTTATATTTGCAGTGTCGAAAGAAACAAAGTAGCCT |
| sequence | GCGATTATCTTTAAATTTTATAAAATGTTAAATTTAAATTAC |
| | GTTAAGATGAAGAAAATGAGATTCTTATTGGTTTTATTGGT |
| | GGGGATATCGATTGTCTCTTGTACTAATGACTTAGATGAAA |
| | TGCTAGGGACTCAAAATTTGCAAACCCGTTCTGTTGAGCCT |
| | AGATTCACAGGAAATATATCAGAAAGTCAGGCGATTGCATT |
| | AATGAATAAATTTCTGGATGCACCCTCTTTTTTTTATTTTGA |
| | AGATGTCATAGGCTTCGATCCGACAACATCTATTTCCACAG |
| | GAAGTTTCACTGTCAACAGAAATGGGAAAACTATACATGTA |
| | CGTTCTATTCAAGTTGGTGTAATAGGAAAGAACGGAAAATA |
| | TGGAGTTTATGGAACTCCAGTAACGAGCAAAGAATTCTATT |
| | CATTGAGAAATGGGATGGTTTTGCATGGAGAACCTATTTAT |
| | GAAGAGAATGATGTAGAAAGAGCCAACCCAGTAGGAATGC |
| | GTTATTACTATACAACTAATGATGTAAATCATAGTCAAAAT |
| | TGCTGGATTGCGATCAATTGGGCTGATGCTTCTACATTTGAA |
| | AGTATAATTGGTTGGGGAGGATCCGGCGGAGGGTCACGTGC |
| | GACGGTTTTTACTCTGGAAGATTTTGTTGGCGATTGGCGTCA |
| | GACCGCGGGTTATAATTTGGATCAAGTCCTGGAACAGGGTG |
| | GCGTAAGCTCTCTGTTCCAGAACCTGGGTGTGAGCGTGACG |
| | CCGATTCAGCGCATCGTTCTGTCCGGCGAGAACGGTCTGAA |
| | AATTGATATTCATGTGATCATCCCGTACGAAGGCCTGAGCG |
| | GTGACCAAATGGGTCAAATCGAGAAAATCTTTAAAGTCGTC |
| | TACCCAGTTGACGATCACCACTTCAAGGTTATCTTGCATTAC |
| | GGTACGCTGGTGATTGATGGTGTGACCCCGAATATGATTGA |
| | CTATTTCGGCCGTCCGTATGAAGGCATTGCCGTTTTTGACGG |
| | TAAAAAGATCACCGTCACCGGTACCCTGTGGAATGGCAATA |
| | AGATTATTGACGAGCGTCTGATTAACCCGGACGGCAGCCTG |
| | CTGTTCCGCGTGACCATCAACGGTGTCACGGGTTGGCGTCT |
| | GTGCGAGCGCATCCTGGCACCGCAGCCGTAAGGTTCCTAAG |
| | GCCATCCTGACGGATGGCCTTTTTTTTGACTGCTATGACTTG |
| | ATACCGGCTATTACGAGCGCTTAAACGGCGCGCCTGATAGG |
| | TGGGCTGCCCTTCCTGGTTGGCTTGGTTTCATCAGCCATCCG |
| | CTTGCCCTCATCTGTTACGCCGGCGGTAGCCGGCCAGCCTC |
| | GCAGAGCAGGATTCCCGTTGAGCACCGCCAGGTGCGAATAA |
| | GGGACAGTGAAGAAGGAACACCCGCTCGCGGGTGGGCCTA |
| | CTTCACCTATCCTGCCCGGCTGACGCCGTTGGATACACCAA |
| | GGAAAGTCTACACGAACCCTTTGGCAAAATCCTGTATATCG |
| | TGCGAAAAAGGATGGATATACCGAAAAAATCGCTATAATG |
| | ACCCCGAAGCAGGGTTATGCAGCGGAAAAGCGGGATTAAA |
| | AGTCGGGGATTGGTGAACAAAAAGGTGTTTCTCTCTTTAAG |
| | AGAAATATCGTTTTGCTAAACAGTTGATATTGAGGTATCATT |
| | TTATCGTAAAAGACATTTTTGCTCAACAATTGCTTGACGGA |
| | AATCAACAAATTTTAGCATTTTGTAAAAAAGTCGCTATATA |
| | ATTTGGTGAATTGGAGTTATTTTCATATTTTTGCATCCCGAA |
| | GAGTTTCTCTTAAAGAGAGAAACATCTTTTGCATACCTTTTC |
| | CGACCGAATTTTTATGTCGTAAAGAGGGGCTTTGCAGGGGG |
| | TGGACTCAGAAAGATGAGAATAGATGACTATTGTAGTTGAA |
| | ACACATAGAAAGTTGCTGATATACAGACCGATACGCATATC |
| | GGGATGAACCATGAGTACGTTCTTTTCTCAAAAAACATAAA |
| | TATTCGAAAAGAGATGCAATAAATTAAGGAGAGGTTATAAT |
| | GAACAAAGTAAATATAAAAGATAGTCAAAATTTTATTACTT |
| | CAAAATATCACATAGAAAAAATAATGAATTGCATAAGTTTA |
| | GATGAAAAAGATAACATCTTTGAAATAGGTGCAGGGAAAG |
| | GTCATTTTACTGCTGGATTGGTAAAGAGATGTAATTTTGTAA |
| | CGGCGATAGAAATTGATTCTAAATTATGTGAGGTAACTCGT |
| | AATAAGCTCTTAAATTATCCTAACTATCAAATAGTAAATGA |
| | TGATATACTGAAATTTACATTTCCTAGCCACAATCCATATAA |
| | AATATTTGGCAGCATACCTTACAACATAAGCACAAATATAA |
| | TTCGAAAAATTGTTTTTGAAAGTTCAGCCACAATAAGTTATT |
| | TAATAGTGGAATATGGTTTTGCTAAAATGTTATTAGATACA |
| | AACAGATCACTAGCATTGCTGTTAATGGCAGAGGTAGATAT |
| | TTCTATATTAGCAAAAATTCCTAGGTATTATTTCCATCCAAA |
| | ACCTAAAGTGGATAGCACATTAATTGTATTAAAAAGAAAGC |
| | CAGCAAAAATGGCATTTAAAGAGAGAAAAAAATATGAAAC |
| | TTTTGTAATGAAATGGGTTAACAAAGAGTACGAAAAACTGT |
| | TTACAAAAAATCAATTTAATAAAGCTTTAAAACATGCGAGA |
| | ATATATGATATAAACAATATTAGTTTCGAACAATTTGTATCG |
| | CTATTTAATAGTTATAAAATATTTAACGGCTAAAAACAATA |
| | GGCCACATGCAACTGTAAATGTTTACGCGGGTACCGACACC |
| | GCGGTGGAGGGGAATTGTGTTACAACCAATTAACCAATTCT |
| | GATTAGAAAAACTCATCGAGCATCAAATGAAACTGCAATTT |
| | ATTCATATCAGGATTATCAATACCATATTTTTGAAAAAGCC |
| | GTTTCTGTAATGAAGGAGAAAACTCACCGAGGCAGTTCCAT |
| | AGGATGGCAAGATCCTGGTATCGGTCTGCGATTCCGACTCG |
| | TCCAACATCAATACAACCTATTAATTTCCCCTCGTCAAAAAT |
| | AAGGTTATCAAGTGAGAAATCACCATGAGTGACGACTGAAT |
| | CCGGTGAGAATGGCAAAAGCTTATGCATTTCTTTCCAGACT |
| | TGTTCAACAGGCCAGCCATTACGCTCGTCATCAAAATCACT |
| | CGCATCAACCAAACCGTTATTCATTCGTGATTGCGCCTGAG |
| | CGAGGCGAAATACGCGATCGCTGTTAAAAGGACAATTACA |
| | AACAGGAATCGAATGCAACCGGCGCAGGAACACTGCCAGC |
| | GCATCAACAATATTTTCACCTGAATCAGGATATTCTTCTAAT |
| | ACCTGGAATGCTGTTTTCCCGGGGATCGCAGTGGTGAGTAA |
| | CCATGCATCATCAGGAGTACGGATAAAATGCTTGATGGTCG |
| | GAAGAGGCATAAATTCCGTCAGCCAGTTTAGTCTGACCATC |
| | TCATCTGTAACATCATTGGCAACGCTACCTTTGCCATGTTTC |
| | AGAAACAACTCTGGCGCATCGGGCTTCCCATACAATCGATA |
| | GATTGTCGCACCTGATTGCCCGACATTATCGCGAGCCCATTT |
| | ATACCCATATAAATCAGCATCCATGTTGGAATTTAATCGCG |
| | GCCTGGAGCAAGACGTTTCCCGTTGAATATGGCTCATAACA |
| | CCCCTTGTATTACTGTTTATGTAAGCAGACAGTTTTATTGTT |
| | CATGATGATATATTTTTATCTTGTGCAATGTAACATCAGAGA |
| | TTTTGAGACACAACGTGGCTTTGTTGAATAAATCGAACTTTT |
| | GCTGAGTTGAAGGATCAGGTCATTGGTAACTATCTATGAAA |
| | CTGTTTGATACTTTTATAGTTGATTAAACTTGTTCATGGCAT |
| | TTGCCTTAATATCATCCGCTATGTCAATGTAGGGTTTCATAG |
| | CTTTGTAGTCGCTGTGTCCCGTCCATTTCATGACCACCTGTG |
| | CCGGGATTCCGAGAGCCAGCGCATTGCAGATGAATGTCCTT |
| | TTTCCTGCATGGGTACTGAGCAAAGCGTATTTGGGTGTGAC |
| | TTCATCAATACGTTCATTTCCCTTGTAGTAGGTTTCCCGTAC |
| | AGGCTCGTTGATTTCTGCCAGTTCGCCCAGCTCTTTCAGGTA |
| | ATCGTTCATCTTCTGGTTGCTGATGACGGGCAGAGCCATGT |
| | AATTCTCGAAATGGATGTCCTTGTATTTGTCCAGTATGGCTT |
| | TGCTGTATTTGTTCAGTTCAATCGTCAGGCTGTCGGCAGTCT |
| | TGACTGTGGTTATTTCGATGTGGTCGGACTTCACATCGCTTC |
| | TTTTCAGATTGCGAACATCCGAATACCGCAAACTCGTAAAG |
| | CAGCAGAACAGGAAAACATCACGCACACGTTCCAGGTATTG |
| | CTTATCCTTGGGTATCTGGTAGTCTTTCAGCTTGTTCAGTTC |
| | ATCCCAAGTCAGGAAGATTACTTTTTTCGAGGTGGTTTTCAG |
| | TTTCGGTTTGAACGTATCGTATGCAATGTTCTGATGATGTCC |
| | TTTCTTGAAGCTCCAGCGCAGGAACCATTTGAGGAATCCCA |
| | TTTGCTTGCCGATGGTGCTGTTTCTCATATCCTTGGTGTCAC |
| | GCAGGAAGTTGACGTATTCGTTCAATCCAAACTCGTTGAAA |
| | TAGTTGAACGTTGCATCCTCCTTGAACTCTTTGAGGTGGTTC |
| | CTCACTGCTGCAAATTTTTCATAGGTGGATGCCGTCCAGTTA |
| | TTCTGGTTACCGCACTCTTTTACAAACTCATCGAACACCTCC |
| | CAAAAGCTGACAGGGGCTTCTTCCGGCTGTTCTTCACTGGT |
| | ATCTTTCATTCTCATGTTGAAAGCTTCCTTCAACTGTTGGGT |
| | CGTTGGCATGACCTCCTGCACCTCAAATTCCTTGAAAATATT |
| | CTGGATTTCGGCATAGTATTTCAGCAAGTCCGTATTGATTTC |
| | GGCTGCACTTTGCTTTAGCTTGTTGGTACATCCGTTCTTTAC |
| | CCGCTGCTTATCTGCATCCCATTTGGCTACGTCAATCCGGTA |
| | GCCCGTTGTAAACTCGATACGTTGGCTGGCAAAGATGACAC |
| | GCATACGGATGGGTACGTTCTCTACGATTGGCACACCGTTC |
| | TTTTTCCGGCTCTCCAATGCAAAAATGATGTTGCGCTTGATA |
| | TTCATAATTGGGTGCGTTTGAAATTCTACACCCAAATATACA |
| | CCCAATTATTGAGATAGCAAAAGACATTTAGAAACATTTAC |
| | TTTTACTCTATATTGTAATTTACACTTGATTATCAGTCGTTTG |
| | CAGTCTTATGATATTCTGTGAAAGTATAAGTTCGAGAGCCT |
| | GTCTCTCCGCAAAAAACGCTGAAAATCAGCAGATTGCAAAA |
| | CAAACACCCTGTTTTACACCCAAGAATGTAAAGTCGGGTGT |
| | TTTTGTTTTATTTAAGATAATACAACCACTACATAATAAAAG |
| | AGTAGCGATATTAAAAGAATCCGATGAGAAAAGACTAATA |
| | TTTATCTATCCATTCAGTTTGATTTCTCAGGACTTTACATCGT |
| | CCTGAAAGTATTTGTTGCAGTTCAACCTGTTGATAGTACGTA |
| | CTAAGCTCTCATGTTTCACGTACTAAGCTCTCATGTTTAACG |
| | TACTAAGCTCTCATGTTTAACGAACTAAACCCTCATGGCTA |
| | ACGTACTAAGCTCTCATGGCTAACGTACTAAGCTCTCATGTT |
| | TCACGTACTAAGCTCTCATGTTTGAACAATAAAATTAATAT |
| | AAATCAGCAACTTAAATAGCCTCTAAGGTTTTAAGTTTTATA |
| | AGAAAAAAAAGAATATATAAGGCTTTTAAAGCTTTTAAGGT |
| | TTAACGGTTGTGGACAACAAGCCAGGGATGTAACGCACTGA |
| | GAAGCCCTTAGAGCCTCTCAAAGCAATTTTGAGTGACACAG |
| | GAACACTTAACGGCTGACATGGGGCGGCCGCACGACGTACC |
| | GGACTCAGTAGGGAGAGCTGTATGTGGGTAG |
| SEQ ID NO: 47- | GATAAAACGAAAGGCTCAGTCGAAAGACTGGGCCTTTCGTT |
| sZR1064; 1E6-4469- | TTACAATTGGGCTACCTTTTTTTTGTTTTGTTTGCAATGGTTA |
| P5B10-GGSGG-RAT- | ATCTATTGTTAAAATTTAAAGTTTCACTTGAACTTTCAAATA |
| NL-NSQ! nucleotide | ATGTTCTTATATTTGCAGTGTCGAAAGAAACAAAGTAGCCT |
| sequence | GCGATTATCTTTAAATTTTATAAAATGTTAAATTTAAATTAC |
| | GTTAAGATGAAGAAAATGAGATTCTTATTGGTTTTATTGGT |
| | GGGGATATCGATTGTCTCTTGTACTAATGACTTAGATGAAA |
| | TGCTAGGGACTCAAAATTTGCAAACCCGTTCTGTTGAGCCT |
| | AGATTCACAGGAAATATATCAGAAAGTCAGGCGATTGCATT |
| | AATGAATAAATTTCTGGATGCACCCTCTTTTTTTTATTTTGA |
| | AGATGTCATAGGCTTCGATCCGACAACATCTATTTCCACAG |
| | GAAGTTTCACTGTCAACAGAAATGGGAAAACTATACATGTA |
| | CGTTCTATTCAAGTTGGTGTAATAGGAAAGAACGGAAAATA |
| | TGGAGTTTATGGAACTCCAGTAACGAGCAAAGAATTCTATT |
| | CATTGAGAAATGGGATGGTTTTGCATGGAGAACCTATTTAT |
| | GAAGAGAATGATGTAGAAAGAGCCAACCCAGTAGGAATGC |
| | GTTATTACTATACAACTAATGATGTAAATCATAGTCAAAAT |
| | TGCTGGATTGCGATCAATTGGGCTGATGCTTCTACATTTGAA |
| | AGTATAATTGGTTGGGGAGGATCCGGCGGAGGGTCACGTGC |
| | GACGGTTTTTACTCTGGAAGATTTTGTTGGCGATTGGCGTCA |
| | GACCGCGGGTTATAATTTGGATCAAGTCCTGGAACAGGGTG |
| | GCGTAAGCTCTCTGTTCCAGAACCTGGGTGTGAGCGTGACG |
| | CCGATTCAGCGCATCGTTCTGTCCGGCGAGAACGGTCTGAA |
| | AATTGATATTCATGTGATCATCCCGTACGAAGGCCTGAGCG |
| | GTGACCAAATGGGTCAAATCGAGAAAATCTTTAAAGTCGTC |
| | TACCCAGTTGACGATCACCACTTCAAGGTTATCTTGCATTAC |
| | GGTACGCTGGTGATTGATGGTGTGACCCCGAATATGATTGA |
| | CTATTTCGGCCGTCCGTATGAAGGCATTGCCGTTTTTGACGG |
| | TAAAAAGATCACCGTCACCGGTACCCTGTGGAATGGCAATA |
| | AGATTATTGACGAGCGTCTGATTAACCCGGACGGCAGCCTG |
| | CTGTTCCGCGTGACCATCAACGGTGTCACGGGTTGGCGTCT |
| | GTGCGAGCGCATCCTGGCAAATTCGCAGTAAGGTTCCTAAG |
| | GCCATCCTGACGGATGGCCTTTTTTTTGACTGCTATGACTTG |
| | ATACCGGCTATTACGAGCGCTTAAACGGCGCGCCTGATAGG |
| | TGGGCTGCCCTTCCTGGTTGGCTTGGTTTCATCAGCCATCCG |
| | CTTGCCCTCATCTGTTACGCCGGCGGTAGCCGGCCAGCCTC |
| | GCAGAGCAGGATTCCCGTTGAGCACCGCCAGGTGCGAATAA |
| | GGGACAGTGAAGAAGGAACACCCGCTCGCGGGTGGGCCTA |
| | CTTCACCTATCCTGCCCGGCTGACGCCGTTGGATACACCAA |
| | GGAAAGTCTACACGAACCCTTTGGCAAAATCCTGTATATCG |
| | TGCGAAAAAGGATGGATATACCGAAAAAATCGCTATAATG |
| | ACCCCGAAGCAGGGTTATGCAGCGGAAAAGCGGGATTAAA |
| | AGTCGGGGATTGGTGAACAAAAAGGTGTTTCTCTCTTTAAG |
| | AGAAATATCGTTTTGCTAAACAGTTGATATTGAGGTATCATT |
| | TTATCGTAAAAGACATTTTTGCTCAACAATTGCTTGACGGA |
| | AATCAACAAATTTTAGCATTTTGTAAAAAAGTCGCTATATA |
| | ATTTGGTGAATTGGAGTTATTTTCATATTTTTGCATCCCGAA |
| | GAGTTTCTCTTAAAGAGAGAAACATCTTTTGCATACCTTTTC |
| | CGACCGAATTTTTATGTCGTAAAGAGGGGCTTTGCAGGGGG |
| | TGGACTCAGAAAGATGAGAATAGATGACTATTGTAGTTGAA |
| | ACACATAGAAAGTTGCTGATATACAGACCGATACGCATATC |
| | GGGATGAACCATGAGTACGTTCTTTTCTCAAAAAACATAAA |
| | TATTCGAAAAGAGATGCAATAAATTAAGGAGAGGTTATAAT |
| | GAACAAAGTAAATATAAAAGATAGTCAAAATTTTATTACTT |
| | CAAAATATCACATAGAAAAAATAATGAATTGCATAAGTTTA |
| | GATGAAAAAGATAACATCTTTGAAATAGGTGCAGGGAAAG |
| | GTCATTTTACTGCTGGATTGGTAAAGAGATGTAATTTTGTAA |
| | CGGCGATAGAAATTGATTCTAAATTATGTGAGGTAACTCGT |
| | AATAAGCTCTTAAATTATCCTAACTATCAAATAGTAAATGA |
| | TGATATACTGAAATTTACATTTCCTAGCCACAATCCATATAA |
| | AATATTTGGCAGCATACCTTACAACATAAGCACAAATATAA |
| | TTCGAAAAATTGTTTTTGAAAGTTCAGCCACAATAAGTTATT |
| | TAATAGTGGAATATGGTTTTGCTAAAATGTTATTAGATACA |
| | AACAGATCACTAGCATTGCTGTTAATGGCAGAGGTAGATAT |
| | TTCTATATTAGCAAAAATTCCTAGGTATTATTTCCATCCAAA |
| | ACCTAAAGTGGATAGCACATTAATTGTATTAAAAAGAAAGC |
| | CAGCAAAAATGGCATTTAAAGAGAGAAAAAAATATGAAAC |
| | TTTTGTAATGAAATGGGTTAACAAAGAGTACGAAAAACTGT |
| | TTACAAAAAATCAATTTAATAAAGCTTTAAAACATGCGAGA |
| | ATATATGATATAAACAATATTAGTTTCGAACAATTTGTATCG |
| | CTATTTAATAGTTATAAAATATTTAACGGCTAAAAACAATA |
| | GGCCACATGCAACTGTAAATGTTTACGCGGGTACCGACACC |
| | GCGGTGGAGGGGAATTGTGTTACAACCAATTAACCAATTCT |
| | GATTAGAAAAACTCATCGAGCATCAAATGAAACTGCAATTT |
| | ATTCATATCAGGATTATCAATACCATATTTTTGAAAAAGCC |
| | GTTTCTGTAATGAAGGAGAAAACTCACCGAGGCAGTTCCAT |
| | AGGATGGCAAGATCCTGGTATCGGTCTGCGATTCCGACTCG |
| | TCCAACATCAATACAACCTATTAATTTCCCCTCGTCAAAAAT |
| | AAGGTTATCAAGTGAGAAATCACCATGAGTGACGACTGAAT |
| | CCGGTGAGAATGGCAAAAGCTTATGCATTTCTTTCCAGACT |
| | TGTTCAACAGGCCAGCCATTACGCTCGTCATCAAAATCACT |
| | CGCATCAACCAAACCGTTATTCATTCGTGATTGCGCCTGAG |
| | CGAGGCGAAATACGCGATCGCTGTTAAAAGGACAATTACA |
| | AACAGGAATCGAATGCAACCGGCGCAGGAACACTGCCAGC |
| | GCATCAACAATATTTTCACCTGAATCAGGATATTCTTCTAAT |
| | ACCTGGAATGCTGTTTTCCCGGGGATCGCAGTGGTGAGTAA |
| | CCATGCATCATCAGGAGTACGGATAAAATGCTTGATGGTCG |
| | GAAGAGGCATAAATTCCGTCAGCCAGTTTAGTCTGACCATC |
| | TCATCTGTAACATCATTGGCAACGCTACCTTTGCCATGTTTC |
| | AGAAACAACTCTGGCGCATCGGGCTTCCCATACAATCGATA |
| | GATTGTCGCACCTGATTGCCCGACATTATCGCGAGCCCATTT |
| | ATACCCATATAAATCAGCATCCATGTTGGAATTTAATCGCG |
| | GCCTGGAGCAAGACGTTTCCCGTTGAATATGGCTCATAACA |
| | CCCCTTGTATTACTGTTTATGTAAGCAGACAGTTTTATTGTT |
| | CATGATGATATATTTTTATCTTGTGCAATGTAACATCAGAGA |
| | TTTTGAGACACAACGTGGCTTTGTTGAATAAATCGAACTTTT |
| | GCTGAGTTGAAGGATCAGGTCATTGGTAACTATCTATGAAA |
| | CTGTTTGATACTTTTATAGTTGATTAAACTTGTTCATGGCAT |
| | TTGCCTTAATATCATCCGCTATGTCAATGTAGGGTTTCATAG |
| | CTTTGTAGTCGCTGTGTCCCGTCCATTTCATGACCACCTGTG |
| | CCGGGATTCCGAGAGCCAGCGCATTGCAGATGAATGTCCTT |
| | TTTCCTGCATGGGTACTGAGCAAAGCGTATTTGGGTGTGAC |
| | TTCATCAATACGTTCATTTCCCTTGTAGTAGGTTTCCCGTAC |
| | AGGCTCGTTGATTTCTGCCAGTTCGCCCAGCTCTTTCAGGTA |
| | ATCGTTCATCTTCTGGTTGCTGATGACGGGCAGAGCCATGT |
| | AATTCTCGAAATGGATGTCCTTGTATTTGTCCAGTATGGCTT |
| | TGCTGTATTTGTTCAGTTCAATCGTCAGGCTGTCGGCAGTCT |
| | TGACTGTGGTTATTTCGATGTGGTCGGACTTCACATCGCTTC |
| | TTTTCAGATTGCGAACATCCGAATACCGCAAACTCGTAAAG |
| | CAGCAGAACAGGAAAACATCACGCACACGTTCCAGGTATTG |
| | CTTATCCTTGGGTATCTGGTAGTCTTTCAGCTTGTTCAGTTC |
| | ATCCCAAGTCAGGAAGATTACTTTTTTCGAGGTGGTTTTCAG |
| | TTTCGGTTTGAACGTATCGTATGCAATGTTCTGATGATGTCC |
| | TTTCTTGAAGCTCCAGCGCAGGAACCATTTGAGGAATCCCA |
| | TTTGCTTGCCGATGGTGCTGTTTCTCATATCCTTGGTGTCAC |
| | GCAGGAAGTTGACGTATTCGTTCAATCCAAACTCGTTGAAA |
| | TAGTTGAACGTTGCATCCTCCTTGAACTCTTTGAGGTGGTTC |
| | CTCACTGCTGCAAATTTTTCATAGGTGGATGCCGTCCAGTTA |
| | TTCTGGTTACCGCACTCTTTTACAAACTCATCGAACACCTCC |
| | CAAAAGCTGACAGGGGCTTCTTCCGGCTGTTCTTCACTGGT |
| | ATCTTTCATTCTCATGTTGAAAGCTTCCTTCAACTGTTGGGT |
| | CGTTGGCATGACCTCCTGCACCTCAAATTCCTTGAAAATATT |
| | CTGGATTTCGGCATAGTATTTCAGCAAGTCCGTATTGATTTC |
| | GGCTGCACTTTGCTTTAGCTTGTTGGTACATCCGTTCTTTAC |
| | CCGCTGCTTATCTGCATCCCATTTGGCTACGTCAATCCGGTA |
| | GCCCGTTGTAAACTCGATACGTTGGCTGGCAAAGATGACAC |
| | GCATACGGATGGGTACGTTCTCTACGATTGGCACACCGTTC |
| | TTTTTCCGGCTCTCCAATGCAAAAATGATGTTGCGCTTGATA |
| | TTCATAATTGGGTGCGTTTGAAATTCTACACCCAAATATACA |
| | CCCAATTATTGAGATAGCAAAAGACATTTAGAAACATTTAC |
| | TTTTACTCTATATTGTAATTTACACTTGATTATCAGTCGTTTG |
| | CAGTCTTATGATATTCTGTGAAAGTATAAGTTCGAGAGCCT |
| | GTCTCTCCGCAAAAAACGCTGAAAATCAGCAGATTGCAAAA |
| | CAAACACCCTGTTTTACACCCAAGAATGTAAAGTCGGGTGT |
| | TTTTGTTTTATTTAAGATAATACAACCACTACATAATAAAAG |
| | AGTAGCGATATTAAAAGAATCCGATGAGAAAAGACTAATA |
| | TTTATCTATCCATTCAGTTTGATTTCTCAGGACTTTACATCGT |
| | CCTGAAAGTATTTGTTGCAGTTCAACCTGTTGATAGTACGTA |
| | CTAAGCTCTCATGTTTCACGTACTAAGCTCTCATGTTTAACG |
| | TACTAAGCTCTCATGTTTAACGAACTAAACCCTCATGGCTA |
| | ACGTACTAAGCTCTCATGGCTAACGTACTAAGCTCTCATGTT |
| | TCACGTACTAAGCTCTCATGTTTGAACAATAAAATTAATAT |
| | AAATCAGCAACTTAAATAGCCTCTAAGGTTTTAAGTTTTATA |
| | AGAAAAAAAAGAATATATAAGGCTTTTAAAGCTTTTAAGGT |
| | TTAACGGTTGTGGACAACAAGCCAGGGATGTAACGCACTGA |
| | GAAGCCCTTAGAGCCTCTCAAAGCAATTTTGAGTGACACAG |
| | GAACACTTAACGGCTGACATGGGGCGGCCGCACGACGTACC |
| | GGACTCAGTAGGGAGAGCTGTATGTGGGTAG |
| SEQ ID NO: 48-GB1 | GGAATGGATACGTATAAACTGATACTGAACGGAAAAACCCT |
| nucleotide sequence | GAAAGGAGAAACGACCACTGAAGCTGTAGATGCAGCGACT |
| | GCTGAAAAAGTCTTCAAACAGTACGCTAATGATAATGGTGT |
| | CGACGGAGAATGGACCTATGATGATGCAACTAAAACATTTA |
| | CCGTGACGGAA |
| SEQ ID NO: 49-GB1 | GMDTYKLILNGKTLKGETTTEAVDAATAEKVFKQYANDNGV |
| amino acid sequence | DGEWTYDDATKTFTVTE |
| SEQ ID NO: 50- | GTGGATAATAAGTTCAATAAAGAATTAGGTTGGGCAATTGG |
| Affibody 1 nucleotide | AGAAATTGGCACCCTGCCAAACCTGAATCATCAGCAATTTA |
| sequence | GAGCCTTTATCCTGAGTTTATGGGATGATCCTAGTCAGAGT |
| | GCAAATTTGCTTGCAGAGGCCAAAAAACTGAATGATGCCCA |
| | AGCCCCTAAA |
| SEQ ID NO: 51- | VDNKFNKELGWAIGEIGTLPNLNHQQFRAFILSLWDDPSQSAN |
| Affibody 1 amino acid | LLAEAKKLNDAQAPK |
| sequence | |
| SEQ ID NO: 52-HS2 | GCTACAAAAAAATTACATTTCGAAACATTACAACTTCACGT |
| detox nucleotide | AGGACAGGAACAGCCAGACCCGGCTACGGATGCTCGTGCA |
| sequence | GTTCCTATTTATCAGACTACATCTTATGTATTTCATAACTCG |
| | GCACATGCAGCCGCACGTTTTGGTTTACAAGATCCGGGTAA |
| | TATTTATGGGCGTCTGACTAATTCCACACAAGGTGTTTTTGA |
| | ACAACGTGTGGCTGCATTGGAAGGGGGAGTGGCAGGACTT |
| | GCGGTAGCTTCCGGGGCTGCTGCCATTACTTATGCGTTTCAG |
| | AACATTACTCGTGCAGGTGATCATGTTGTAGCTGCAAAGAC |
| | TATTTACGGAGGTACTTATAACCTGCTGGCGCATACGTTGCC |
| | GACTTATGGTGTGACTGCTACATTTGTGGACCCTGGTGATCT |
| | TTCTAATTTTGAGAAAGCTATTCAGGAGAATACAAAGGCGG |
| | TGTTTATCGAGACTTTGGGGAATCCTAATTCGAATATTATAG |
| | ATATAGAGGCTGTGGCGGAAATAGCACATCGCCATCACATA |
| | CCTTTGATTATTGATAACACTTTTGGTACTCCTTATCTGATTC |
| | GTCCCATCGAGCACGGAGCAGATATTGTGGTACATTCCGCT |
| | ACAAAATTTATTGGTGGCCATGGAACTTCACTGGGTGGCGT |
| | GATTGTTGATTCAGGTAAATTTGACTGGGTGGCATCAGGTA |
| | AATTCCCTCAACTGACAGAATCGGATCCTTGCTATCATGGG |
| | GTGCGTTTTGTAGAGGCGGCAGGTCCGGCTGCTTATGCTGT |
| | CCGTATTCGTGCTATTTTATTGCGTGATACCGGTGCAACTAT |
| | CAGTCCGTTTAATGCTTTTATTCTGTTGCAGGGATTGGAGAC |
| | ACTTTCATTGCGTGTGGAACGTCATGTAGAGAATGCGTTGA |
| | AAGTGGTGGAGCATTTGAAAAATCATCCGAAGATCAAGAA |
| | AGTAAATCACCCTTCCTTGCCGGAACATCCTGACCATGCTCT |
| | GTATCAGCGTTATTTCCCCAATGGAGCTAGTTCTATTTTCAC |
| | TATCGAAGTGAAAGGTGGTCAGGAAGAAGCACATCGTTTCA |
| | TCGATAGTTTGGAAATCTTTTCTTTATTGGCCAATGTCGCCG |
| | ATGTGAAGTCATTGGTTATCCATCCTGCCAGTACCACTCACT |
| | CCCAGTTGAATGCGGAAGAATTGGCGGAACAGGGTATTTAT |
| | CCGGGGACAGTGCGTTTATCAATAGGTACGGAACATATAGA |
| | TGATTTGCTGGCCGATTTGGATCAGGCATTAGCTGGAATT |
| SEQ ID NO: 53-HS2 | ATKKLHFETLQLHVGQEQPDPATDARAVPIYQTTSYVFHNSAH |
| detox amino acid | AAARFGLQDPGNIYGRLTNSTQGVFEQRVAALEGGVAGLAVA |
| sequence | SGAAAITYAFQNITRAGDHVVAAKTIYGGTYNLLAHTLPTYGV |
| | TATFVDPGDLSNFEKAIQENTKAVFIETLGNPNSNIIDIEAVAEI |
| | AHRHHIPLIIDNTFGTPYLIRPIEHGADIVVHSATKFIGGHGTSLG |
| | GVIVDSGKFDWVASGKFPQLTESDPCYHGVRFVEAAGPAAYA |
| | VRIRAILLRDTGATISPFNAFILLQGLETLSLRVERHVENALKVV |
| | EHLKNHPKIKKVNHPSLPEHPDHALYQRYFPNGASSIFTIEVKG |
| | GQEEAHRFIDSLEIFSLLANVADVKSLVIHPASTTHSQLNAEEL |
| | AEQGIYPGTVRLSIGTEHIDDLLADLDQALAGI |
| SEQ ID NO: 54-Fp | GTAGGTAACACTTTCTTGCAGTCTACTATCAACCGTACTATC |
| MAM peptide | GGTGTATTG |
| nucleotide sequence | |
| SEQ ID NO: 55-Fp | VGNTFLQSTINRTIGVL |
| MAM peptide amino | |
| acid sequence | |
| SEQ ID NO: 56-MF7 | ATGTCTTACTCTGCTGGTTTC |
| nucleotide sequence | |
| SEQ ID NO: 57-MF7 | MSYSAGF |
| amino acid sequence | |
| SEQ ID NO: 58-ILRa | CGGCCATCTGGAAAACGTCCTTGTAAGATGCAGGCATTTCG |
| (Mm) nucleotide | TATTTGGGATACGAACCAAAAGACATTCTACCTTCGTAACA |
| sequence | ACCAATTGATCGCTGGTTATCTGCAAGGTCCTAACATCAAA |
| | TTGGAGGAAAAAATCGACATGGTGCCTATTGATTTGCACAG |
| | CGTATTCCTTGGCATACATGGTGGCAAACTGTGTTTATCATG |
| | CGCAAAGTCGGGAGATGATATCAAACTTCAGCTGGAAGAA |
| | GTAAATATAACAGACCTGAGCAAAAACAAGGAAGAAGATA |
| | AACGCTTTACCTTTATACGTTCAGAAAAAGGACCTACCACA |
| | TCTTTCGAGTCAGCTGCATGTCCGGGATGGTTTCTTTGCACA |
| | ACTTTGGAGGCTGATCGCCCGGTGTCTTTAACAAACACGCC |
| | CGAAGAGCCACTTATCGTAACGAAATTTTACTTCCAGGAAG |
| | ACCAA |
| SEQ ID NO: 59-ILRa | RPSGKRPCKMQAFRIWDTNQKTFYLRNNQLIAGYLQGPNIKLE |
| (Mm) amino acid | EKIDMVPIDLHSVFLGIHGGKLCLSCAKSGDDIKLQLEEVNITD |
| sequence | LSKNKEEDKRFTFIRSEKGPTTSFESAACPGWFLCTTLEADRPV |
| | SLTNTPEEPLIVTKFYFQEDQ |
| SEQ ID NO: 60- | GTCCAGTTAGTTGAGAGCGGCGGTGGACTGGTGCAAGCAGG |
| VHH1 anti-TNF | TGGCAGCTTGAGCCTGTCCTGTAGTGCCTCTGGTCGTTCTTT |
| nucleotide sequence | ATCTAATTACTATATGGGCTGGTTCAGACAGGCTCCTGGGA |
| | AGGAGAGAGAGCTGTTAGGCAATATTAGCTGGCGTGGTTAC |
| | AACATTTATTACAAGGACAGTGTGAAAGGTCGCTTTACGAT |
| | TTCGCGGGACGATGCTAAGAACACCATTTACTTACAGATGA |
| | ATAGACTGAAGCCTGAAGACACTGCAGTGTATTACTGTGCT |
| | GCCAGTATTTTGCCTTTGTCCGACGATCCTGGCTGGAACACA |
| | TATTGGGGTCAGGGTACCCAGGTCACAGTGTCT |
| SEQ ID NO: 61- | VQLVESGGGLVQAGGSLSLSCSASGRSLSNYYMGWFRQAPGK |
| VHH1 anti-TNF amino | ERELLGNISWRGYNIYYKDSVKGRFTISRDDAKNTIYLQMNRL |
| acid sequence | KPEDTAVYYCAASILPLSDDPGWNTYWGQGTQVTVS |
| SEQ ID NO: 62- | GGTTCCGGGTCTGGATCA |
| (GS)4 linker nucleotide | |
| sequence | |
| SEQ ID NO: 63- | GSGSGSGS |
| (GS)4 linker amino | |
| acid sequence | |
| SEQ ID NO: 64-CL3 | CCTACAGCGTCCCCTCTC |
| linker nucleotide | |
| sequence | |
| SEQ ID NO: 65-CL3 | PTASPL |
| linker amino acid | |
| sequence | |
| SEQ ID NO: 66- | ATGTTAAATTTAAATTATGTAAAGATGAAAAAAATGAGATT |
| 04469 long nucleotide | TTTATTAGTATTATTAGTGGGTATATCTATTGTATCATGTAC |
| sequence | TAATGACTTAGATGAAATGCTAGGGACTCAAAATTTGCAAA |
| | CCCGTTCTGTTGAGCCTAGATTCACAGGAAATATATCAGAA |
| | AGTCAGGCGATTGCATTAATGAATAAATTTCTGGATGCACC |
| | CTCTTTTTTTTATTTTGAAGATGTCATAGGCTTCGATCCGAC |
| | AACATCTATTTCCACAGGAAGTTTCACTGTCAACAGAAATG |
| | GGAAAACTATACATGTACGTTCTATTCAAGTTGGTGTAATA |
| | GGAAAGAACGGAAAATATGGAGTTTATGGAACTCCAGTAA |
| | CGAGCAAAGAATTCTATTCATTGAGAAATGGGATGGTTTTG |
| | CATGGAGAACCTATTTATGAAGAGAATGATGTAGAAAGAG |
| | CCAACCCAGTAGGAATGCGTTATTACTATACAACTAATGAT |
| | GTAAATCATAGTCAAAATTGCTGGATTGCGATCAATTGGGC |
| | TGATGCTTCTACATTTGAAAGTATAATTGGTTGG |
| SEQ ID NO: 67- | MLNLNYVKMKKMRFLLVLLVGISIVSCTNDLDEMLGTQNLQT |
| 04469 long amino acid | RSVEPRFTGNISESQAIALMNKFLDAPSFFYFEDVIGFDPTTSIST |
| sequence | GSFTVNRNGKTIHVRSIQVGVIGKNGKYGVYGTPVTSKEFYSL |
| | RNGMVLHGEPIYEENDVERANPVGMRYYYTTNDVNHSQNCW |
| | IAINWADASTFESIIGW |

### EXAMPLES

### Example 1

The present Example describes exemplary fusion polypeptides that allow for efficient secretion of soluble, heterologous cargo, e.g., polypeptides, from inside a cell into an extracellular environment.

### Materials & Methods

### Bacterial Strains and Culture Conditions

Experiments were performed using Bacteroides strain NB144, which can controllably colonize a host through use of the seaweed derived polysaccharide porphyran.

Bacteroides strains were grown in BHIS media, comprised of Brain Heart Infusion media (Difco) supplemented with hemin (5 μg/mL), vitamin K (1 μg/mL), and cysteine (0.5 g/mL). Bacteroides strains were grown in an anaerobic chamber (Coy Laboratory Products Inc.) at 37° C. under an atmosphere of 20% C02, 5% H₂, and 85% N₂. When required, the following antibiotics were used for selection at the specified concentrations: erythromycin (25 μg/mL), tetracycline (2 μg/mL), and gentamicin (200 μg/mL). Routine molecular procedures were performed using E. coli. E. coli S17-1λ pir was used for conjugal transfer of genetic material from E. coli to Bacteroides. E. coli strains were routinely grown aerobically in Lysogeny Broth (LB) media with shaking at 250 rpm at 37° C. When appropriate, LB was supplemented with 100 μg/mL carbenicillin.

### Construction of 04469-Cargo Vectors

Heterologous cargo proteins for secretion via a 04469 secretion domain, were synthesized into gBlocks by Integrated DNA Technologies (Newark, NJ). Using Golden Gate cloning, HSDH genes were assembled with the appropriate transcriptional and translational features into a vector suitable for heterologous expression, chromosomal integration, and conjugal transfer to Bacteroides. Vectors were based on the mobilizable Bacteroides element NBU2.

### Luminescence Quantification

To prepare bacterial samples for luminescence signal measurement (e.g., nanoluciferase signal, firefly luciferase signal, etc.) either total bacterial culture volume (sometimes referred to as “culture” or “total”), cell-free bacterial spent supernatants (generated by centrifugation of saturated cultures at 16,000 rcf for 5 min to separate cell biomass from spent media supernatant, referred to as “supernatant” or “extracellular”) or bacterial cell pellets resuspended in 1× volume PBS (“cells” or “pellet”) post-centrifugation were generated. Luminescence was measured according to manufacturer's protocol using the Nano-Glo Luciferase Assay System (Promega) with minor modifications: 5 μl of sample was mixed with 5 μl of buffer/substrate mix, then briefly vortexed before quantification at room temperature in black opaque 384 well plates using the Spark microplate plate reader (Tecan). Firefly luminescence measurements were quantified in a similar method using the Bright-Glo Luciferase Assay System (Promega).

### Molecular Weight Cut-Off Filters

Bacterial cell-free supernatants were applied to 1 ml Amicon® Ultra centrifugal filters of various molecular weight cut-offs (100 kDa, 50 kDa, 30 kDa, and 10 kDa) and utilized according to manufacturer's instructions (Millipore Sigma). Luminescence signal in the filter flow-through was analyzed as described above.

### SDS-PAGE & Western Blot

Lysates of bacterial cell pellets or cell-free supernatants were made using 2× or 4× Laemmli Sample Buffer with 2-mercaptoethanol (Bio-Rad, final concentration 357 mM), boiled at 95 C for 5 min, and then subjected to centrifugation at 16,000 rcf for 5 min. Proteins were separated by electrophoresis using 4-20% Mini-PROTEAN TGX Stain-Free Protein Gels and afterwards transferred to nitrocellulose membrane using the Trans-Blot® Turbo™ Transfer System (Bio-Rad). Blots were blocked for 15 min at room temperature using the EveryBlot Blocking buffer (BioRad). After antibody incubation steps, band intensity was detected via chemiluminescence (Clarity Max ECL, BioRad) and imaged using a Chemi-Doc instrument (Bio-Rad). Nanoluciferase was detected using mouse anti-Nanoluciferase monoclonal antibody (Promega, N7000) at 1:1000 dilution and detected using Anti-Mouse IgG (H+L), HRP Conjugate (Promega, W4021) antibody at 1:3000 dilution. Antibodies were diluted in the EveryBlot Blocking buffer (BioRad) and incubated for 1 hr at room temperature rocking, and washed with TBST (0.05% Tween-20). A single antibody mouse monoclonal ANTI-FLAG® M2-Peroxidase (HRP) was used at 1:1000 dilution to detect FLAG-tagged 04469-cargo fusion protein (Sigma, A8592).

### Mouse Experiments

Conventionally-raised C57BL/6 female mice 6-8 weeks of age were purchased from Charles River and housed using an Innovive Disposable IVC Rodent Caging System (San Diego, CA). Prior to colonization, mice were fed a standard sterilized autoclaved diet ad libitum with free access to water. Housing conditions were at room temperature (24° C.) and 12 h/12 h light/dark cycle (7:00 am-7:00 pm). Mice were acclimated for a minimum of 4 days prior to colonization.

Before colonizing, mice were orally gavaged with 200 L of a 5% (w/v) filter-sterilized solution of sodium bicarbonate in water. 15 minutes after sodium bicarbonate delivery, mice were gavaged with a mid-log growth phase Bacteroides culture (˜109 CFUs/mL) grown anaerobically in BHIS as described above. Following strain gavage, mice were transferred to a chow diet supplemented with porphyran for Bacteroides strain maintenance. Colonization was verified by qPCR on fecal pellets at day 5 post-gavage, using primers specific to a gene present in the porphyran utilization locus.

After 6 days of colonization, n=4 mice were sacrificed from each experimental group, and cecal and fecal samples were harvested for ex vivo analysis of Nanoluciferase signal. Samples were kept at 4 C during processing. Tissue samples were weighed and resuspended in equivalent concentrations (weight/volume) of PBS. An initial slow-speed spin (0.2 RCF, 5 min) was performed to remove insoluble digestive material; supernatant from this spin was used to quantify luminescence in the total tissue contents. A partial volume from the supernatant of the first spin was subjected to a second harder centrifugation (16,000 rcf, 10 min) and supernatant from this spin was deemed the extracellular fraction; the remaining cell pellet was resuspended in 1×PBS volume for analysis of Nanoluciferase signal still present inside microbial cells. Nanoluciferase signal was measured as described above, with the modification that samples were read using white opaque 394 well plates.

### Results

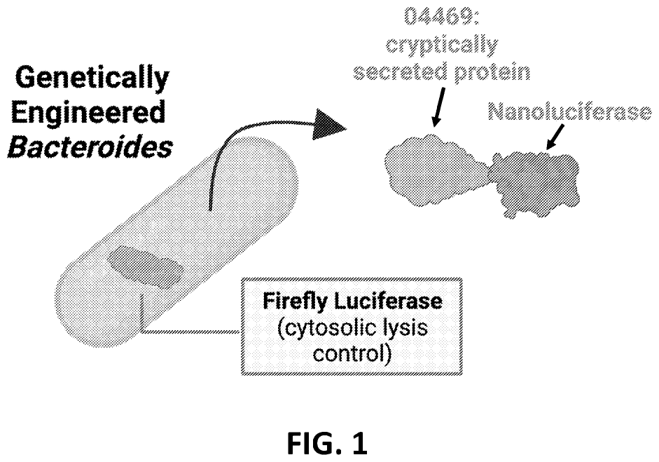

We identified a protein of unknown function we termed “04469,” a bacterial polypeptide that is capable of exporting a tethered heterologous cargo across both membranes of each tested Bacteroides across the genus (see, e.g., FIG. 1). Without wishing to be bound by theory, it is believed that 04469 contains a signal peptide that is recognized by the Sec machinery, which transports the 04469 peptide and its heterologous cargo from the cytoplasm to the periplasm.

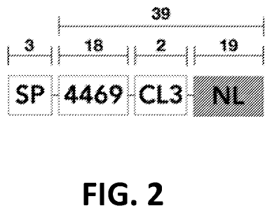

As described herein, an array of heterologous proteins can be secreted from Bacteroides cells when fused to 04469. An exemplary heterologous protein, nanoluciferase, was fused to the C-terminus of 04469 (see, e.g., FIG. 2), and expressed in Bacteroides vulgatus. The strain expressing the 04469 polypeptide fused to nanoluciferase was grown in liquid media (BHIS). After growing to early saturation, the culture was centrifuged to separate the media (supernatant) from the cells (pellet). Luminescence readings were made for the total culture before this centrifugation step, the supernatant, and the pellet. 99% of the luminescence signal measured in the total culture was measured in the supernatant (FIG. 3A). To verify that secretion into the media was not due to an unexpectedly high level of lysis, a cytoplasmically-expressed firefly luciferase (i.e., the firefly luciferase does not have 04469 fused) was also included in this same strain. Luminescence readings from nanoluciferase and firefly luciferase can be orthogonally measured. As expected, very low firefly luminescence was measured in the supernatant fraction, the luminescence remained in the cell pellet fraction (FIG. 3A). A western blot probing with an antibody recognizing nanoluciferase showed that nanoluciferase accumulated in the media supernatant and little remained in the cells (FIG. 3B).

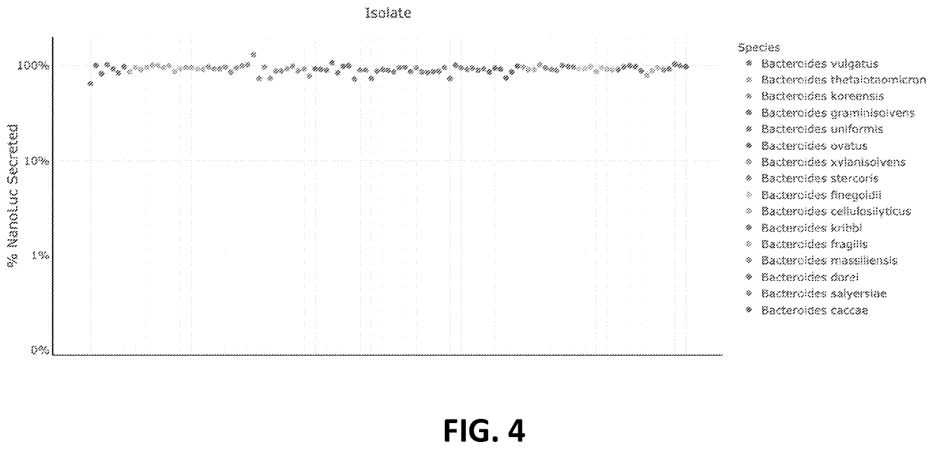

Remarkably, 04469 polypeptides provided herein allow for the secretion of heterologous protein cargo by diverse Bacteroides strains. A diverse set of Bacteroides strains spanning much of the genus were conjugated with the mobilizable element NBU2 containing a heterologous promoter that drives expression of 04469 fused to nanoluciferase. As depicted in FIG. 4, each of this diverse set of Bacteroides exhibited similar high secretion efficiency as in Bacteroides vulgatus, see, e.g., FIGS. 3A-3B.

Protein is Secreted as Soluble Protein, not Associated with OVs

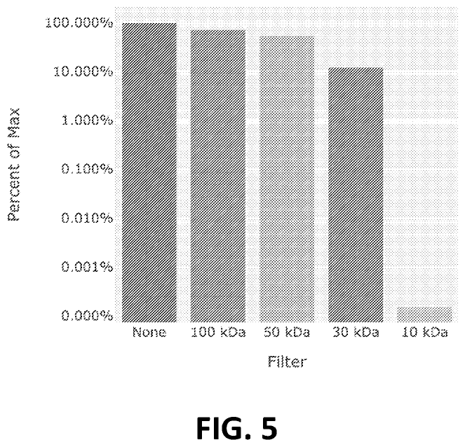

Surprisingly, 04469-mediated secretion results in a soluble protein outside a cell that is not associated with a host cell's membrane or outer membrane vesicles (OMVs). A B. vulgatus strain expressing 04469 fused to nanoluciferase was grown in liquid media (BHIS). The predicted protein product is 40.8 kDa. After growing the culture for approximately 12 hours, the culture was centrifuged to separate the media “supernatant” from the cells. The media was then filtered with various molecular weight cutoff filters (no filter, 100 kDa, 50 kDa, 30 kDa, and 10 kDa) and the luminescence of the supernatant that passed through the filter was measured. Little loss of luminescence was measured until a 30 kDa filter was used, and almost all protein was filtered when a 10 kDa filter was used (see, e.g., FIG. 5).

### Engineered Polypeptides are Capable of Secreting a Variety of Heterologous Protein Cargo In Vitro

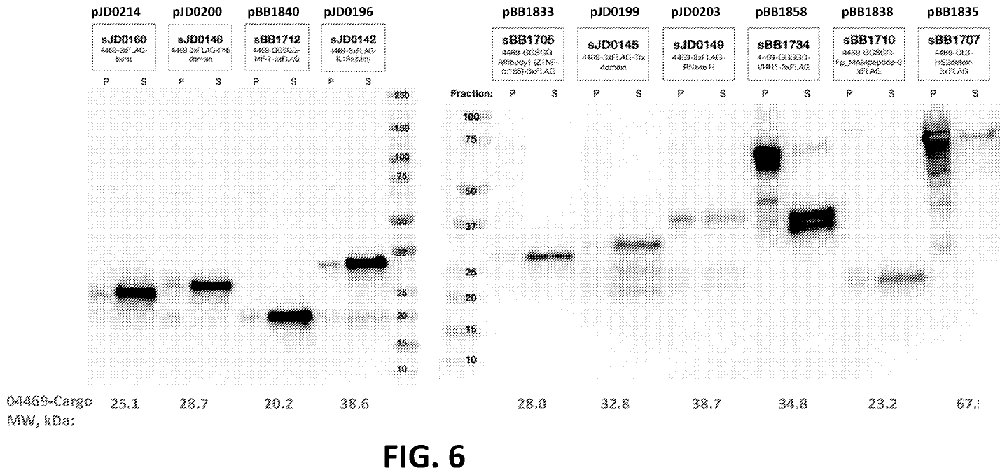

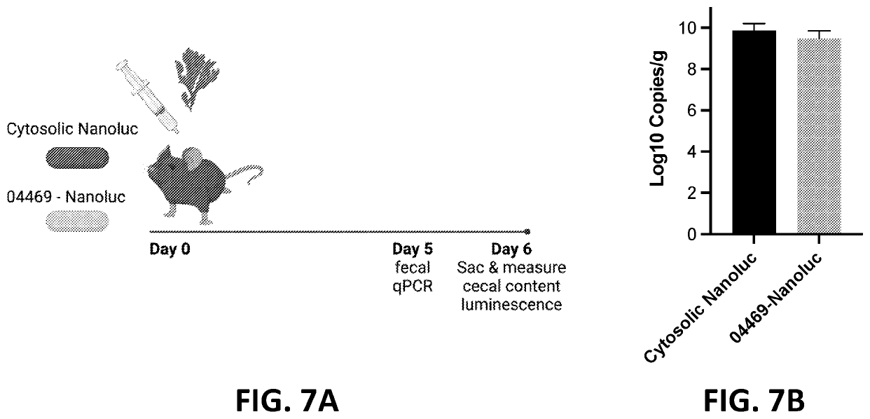

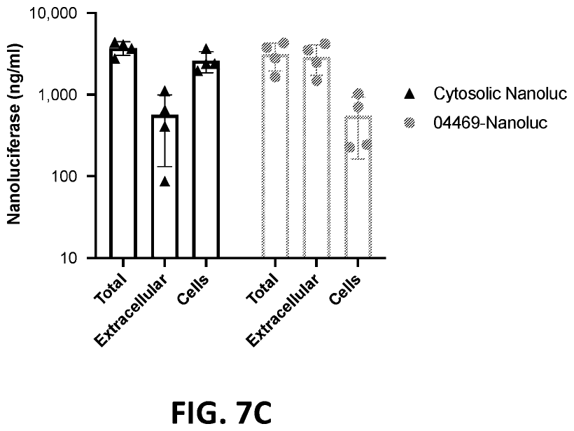

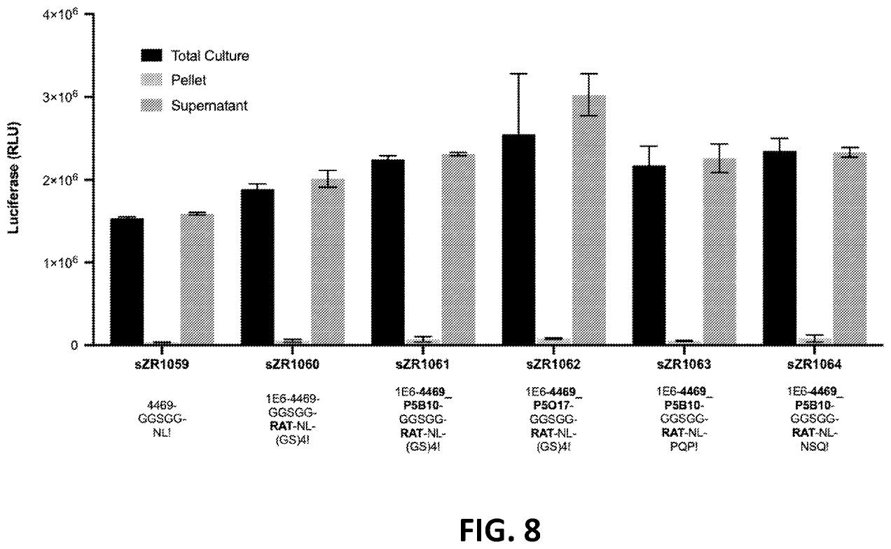

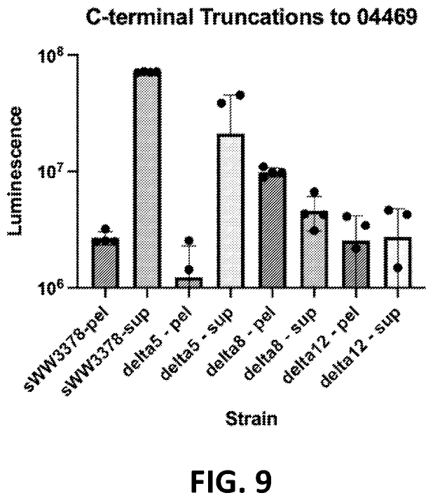

Analyses of various heterologous cargo proteins secreted from engineered Bacteroides cells expressing a recombinant fusion polypeptide, as disclosed herein, were also conducted (see, e.g., FIG. 6). In each case, heterologous cargo proteins were fused to the C-terminus of a 04469 secretion domain. In this example, 04469-3×FLAG-6×His (SEQ ID NOs: 22 and 23), 04469-3×FLAG-Fh8 (SEQ ID NOs: 24 and 25), 04469-GGSGG-MF-7-3×FLAG (SEQ ID NOs: 38 and 39), 04469-3×FLAG-IL1Ra(Mm) (SEQ ID NOs: 26 and 27), 04469-GGSGG-Affibody1 (ZTNFα:185)-3×FLAG (SEQ ID NOs: 28 and 29), 04469-3×FLAG-Trx (SEQ ID NOs: 30 and 31), 04469-3×FLAG-RNaseH (SEQ ID NO: 32 and 33), 04469-GGSGG-VHH1-3×FLAG (SEQ ID NO: 34 and 35), 04469-GGSGG-Fp_MAMpeptide-3×FLAG (SEQ ID NOs: 36 and 37), and 04469-CL3-HS2detox-3×FLAG (SEQ ID NOs: 40 and 41) strains were tested. Epitope tags were fused to each cargo, e.g., a 3×FLAG tag, for visualization by western blot. Predicted sizes of 04469-cargo fusions are listed under each pellet (“P”) or supernatant (“S”) lanes on the western blot for the respective strain tested. This predicted size is assuming the cleavage of the 3.2 kDa predicted signal peptide in the periplasm after transport through the inner membrane via the Sec translocon. As shown, 04469 secretion domains provided herein unexpectedly allow for the secretion of a variety of cargo polypeptides as soluble polypeptides in supernatant.

### Engineered Polypeptides are Capable of Mediating Secretion of Heterologous Proteins in Vivo.

For this example of in vivo secretion using provided engineered polypeptides, conventionally-raised C57BL6 female mice ages 6-8 weeks were engrafted with a Bacteroides vulgatus strain expressing 04469 fused to nanoluciferase (sWD1353). A graphical summary of the study design is presented in FIG. 7A. Both base strains also contain a porphyran polysaccharide utilization loci (PUL) to allow for engraftment in a porphyran concentration dependent manner (see, e.g., WO 2018/112194 A1, and Shepard et al. Nature (2018) 557(7705):434-438). Mice were gavaged on day 1 with a Bacteroides strain that expresses either nanoluciferase alone (cytosolic) or nanoluciferase fused to the C-terminus of 04469 (n=4 per group). Mice were fed chow supplemented with porphyran for the duration of the experiment to allow for porphyran-dependent colonization at high bacterial density.

Colonization density was quantified by qPCR from fecal samples collected on day 5, and indicate comparable levels of bacterial colonization across the two strain groups (FIG. 7B). These data indicate that expression of the 04469-Nanoluciferase fusion protein does not significantly impact in vivo strain fitness.

On day 6 post-colonization, animals were sacrificed and cecal contents were isolated and equivantly diluted in PBS by weight. Cecal contents were centrifuged at low speed to remove large insoluble digestive material; the supernatant from this centrifuge step is labeled as “total” in FIG. 7C. A fraction of this volume was further processed by a high-speed centrifugation step to separate the secreted protein (“extracellular”) from the remaining bacterial cell biomass (“cells”). Similar to what was observed in vitro, nanoluciferase luminescence was observed primarily in the extracellular fraction for the engrafted strain expressing 04469-nanoluciferase, while the luminescence was observed primarily in the cells fraction for the engrafted strain expressing nanoluciferase alone (data not shown). Further, the titer measured was comparable to what was observed from bacterial growth in vitro (FIG. 3A).

### Secretion Domain Variants Offer Improved Secretion of Heterologous Cargo

Various Bacteroides strains expressing a diversity of recombinant polypeptide constructs that include variants of the native 04469 ribosomal binding site (RBS) were tested for enhanced ability to secrete heterologous polypeptides, e.g., nanoluciferase (see, e.g., FIG. 8). Tested strains included base strain sZR1059 (plasmid sequence SEQ ID NO: 42) which included a wild-type RBS, and variant strains sZR1060 (plasmid sequence SEQ ID NO: 43), sZR1061 (plasmid sequence SEQ ID NO: 44), sZR1062 (plasmid sequence SEQ ID NO: 45), sZR1063 (plasmid sequence SEQ ID NO: 46), and sZR1064 (plasmid sequence SEQ ID NO: 47). The total amount of RLUs in FIG. 8 are shown in the total bacterial culture, contained within the bacterial cell pellet (“Pellet”) or the extracellular environment (“Supernatant”), and are depicted for each tested strain. Surprisingly, RBS variants such as “04469_P5B10” and “4469-PO17,” and/or N/C terminal linkers such as “RAT”, “(GS)4!”, “PQP!”, and “NSQ!” demonstrate a marked improvement in secretion of the heterologous nanoluciferase cargo relative to the base strain sZR1059.

### Various C-Terminal Secretion Domain Truncations Maintain Secretion Capability

04469 secretion domain C-terminal truncations were tested for ability to secrete heterologous polypeptides, e.g., nanoluciferase (see, e.g., FIG. 9). Tested strains included base strain sWW3378 (SEQ ID NOs: 9 and 10), and truncations strains delta5-NL (SEQ ID NOs: 11 and 12), in which 5 C-terminal amino acids were deleted, delta 8-NL (SEQ ID NOs: 13 and 14), in which 8 C-terminal amino acids were deleted, and delta 12-NL (SEQ ID NOs: 15 and 16), in which 12 C-terminal amino acids were deleted. The amount of secretion achieved by the various truncation constructs is indicated by comparing the amount of RLU signal in the culture supernatant (“sup”) to the culture bacterial cell pellet (“pel”). Comparison of depicted truncations constructs to full length 04469 shows that up to at least 5 amino acid residues can be deleted from the C-terminus while maintaining secretion of heterologous cargo.

### INCORPORATION BY REFERENCE

The entire disclosure of each of the patent and scientific documents referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

1. A recombinant fusion polypeptide comprising:
(i) a secretion domain comprising an amino acid sequence having at least 80% sequence identity to SEQ ID NO: 4; and
(ii) a heterologous cargo domain.

2. The recombinant fusion polypeptide of claim 1, wherein the secretion domain comprises an amino acid sequence having at least 90% sequence identity to SEQ ID NO: 4.

3. The recombinant fusion polypeptide of claim 1, wherein the secretion domain comprises an amino acid sequence having at least 95% sequence identity to SEQ ID NO: 4.

4. The recombinant fusion polypeptide of claim 1, wherein the secretion domain comprises an amino acid sequence as set forth in SEQ ID NO: 4.

5. The recombinant fusion polypeptide of claim 1, wherein the secretion domain comprises an amino acid sequence having at least 90% sequence identity to SEQ ID NO: 2.

6. The recombinant fusion polypeptide of claim 1, wherein the secretion domain comprises an amino acid sequence having at least 95% sequence identity to SEQ ID NO: 2.

7. The recombinant fusion polypeptide of claim 1, wherein the secretion domain comprises an amino acid sequence as set forth in SEQ ID NO: 2.

8. A recombinant fusion polypeptide comprising:
(i) a secretion domain comprising an amino acid sequence having at least 80% sequence identity to SEQ ID NO: 67; and
(ii) a heterologous cargo domain.

9. The recombinant fusion polypeptide of claim 8, wherein the secretion domain comprises an amino acid sequence having at least 90% sequence identity to SEQ ID NO: 67.

10. The recombinant fusion polypeptide of claim 8, wherein the secretion domain comprises an amino acid sequence having at least 95% sequence identity to SEQ ID NO: 67.

11. The recombinant fusion polypeptide of claim 8, wherein the secretion domain comprises an amino acid sequence as set forth in SEQ ID NO: 67.

12. The recombinant fusion polypeptide of any one of claims 1-11, wherein the heterologous cargo domain comprises an amino acid sequence of about 2 to about 20 amino acids.

13. The recombinant fusion polypeptide of any one of claims 1-11, wherein the heterologous domain comprises an amino acid sequence of about 30 to 40 amino acids.

14. The recombinant fusion polypeptide of any one of claims 1-11, and 13, wherein the heterologous domain comprises an amino acid sequence of about 38 amino acids.

15. The recombinant fusion polypeptide of any one of claims 1-11, wherein the heterologous domain comprises an amino acids sequence of about 450-500 amino acids.

16. The recombinant fusion polypeptide of any one of claims 1-11, and 15, wherein the heterologous domain comprises an amino acid sequence of about 458 amino acids.

17. The recombinant fusion polypeptide of any one of claims 1-16, wherein the heterologous cargo domain comprises a reporter polypeptide.

18. The recombinant fusion polypeptide of claim 17, wherein the reporter polypeptide is selected from the group consisting of: a luciferase (e.g., GFP, RFP, or YFP), horse radish peroxidase (HRP), beta-glucosidase, and variants or combinations thereof.

19. The recombinant fusion polypeptide of claim 18, wherein the luciferase is a nanoluciferase.

20. The recombinant fusion polypeptide of any one of claims 1-19, wherein the recombinant fusion polypeptide comprises an epitope tag.

21. The recombinant fusion polypeptide of claim 20, wherein the epitope tag is also a reporter polypeptide.

22. The recombinant fusion polypeptide of claim 16 or claim 17, wherein the epitope tag is selected from the group consisting of: c-Myc, human influenza hemagglutinin (HA), FLAG, 3×FLAG, 6×His, glutathione-S-transferase (GST), maltose binding protein (MBP), GFP, RFP, mCherry, or variants or combinations thereof.

23. The recombinant fusion polypeptide of any one of claims 1-22, wherein the heterologous cargo domain comprises a bioactive moiety.

24. The recombinant fusion polypeptide of any one of claims 1-23, wherein the heterologous cargo domain comprises a therapeutic polypeptide.

25. The recombinant fusion polypeptide of claim 24, wherein the therapeutic polypeptide is a cytokine, hormone, antibody, affibody, enzyme, bioactive peptide, or derivatives or functional fragments thereof.

26. The recombinant fusion polypeptide of any one of claims 1-25, wherein the heterologous cargo domain is fused directly or indirectly to the C-terminus of the secretion domain.

27. The recombinant fusion polypeptide of any one of claims 1-26, wherein the recombinant fusion polypeptide comprises a linker domain that links the secretion domain to the heterologous cargo domain.

28. The recombinant fusion polypeptide of claim 27, wherein the linker domain comprises a CL3 linker, a (GS)4 linker, a PQP linker, a NSQ linker, or a RAT linker.

29. The recombinant fusion polypeptide of claim 27 or claim 28, wherein the linker domain comprises a protease cleavage site.

30. The recombinant fusion polypeptide of claim 29, wherein the protease cleavage site is cleaved by one or more proteases that are present in the colon of a subject.

31. The recombinant fusion polypeptide of claim 29, wherein the protease cleavage site is cleaved by a protease selected from the group consisting of: chymotrypsin, chymotrypsin-like elastases, trypsin, tobacco etch virus (TEV), thrombin, human neutrophil elastase, cathepsin G, tryptase, chymase, proteinase 3, commensal enteric microbial proteases, and combinations thereof.

32. A polynucleotide encoding the recombinant fusion polypeptide of any one of claims 1-31.

33. A polynucleotide comprising a nucleotide sequence having at least 80% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 18, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 43, SEQ ID NO: 44, SEQ ID NO: 45, SEQ ID NO: 46, SEQ ID NO: 47, or SEQ ID NO: 66.

34. The polynucleotide of claim 33, wherein the polynucleotide comprises a nucleotide sequence having at least 90% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 18, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 43, SEQ ID NO: 44, SEQ ID NO: 45, SEQ ID NO: 46, SEQ ID NO: 47, or SEQ ID NO: 66.

35. The polynucleotide of claim 33, wherein the polynucleotide comprises a nucleotide sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 18, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 43, SEQ ID NO: 44, SEQ ID NO: 45, SEQ ID NO: 46, SEQ ID NO: 47, or SEQ ID NO: 66.

36. The polynucleotide of any one of claims 32-35, wherein the polynucleotide is operably linked to a promoter.

37. The polynucleotide of claim 36, wherein the promoter is an inducible promoter.

38. The polynucleotide of claim 36, wherein the promoter is a constitutive promoter.

39. A vector comprising the polynucleotide of any one of claims 32-38.

40. A Bacteroides cell comprising the polynucleotide of any one of claims 32-38, or the vector of claim 39.

41. The Bacteroides cell of claim 40, wherein the Bacteroides cell is a Bacteroides vulgatus, Bacteroides thetaiotaomicron, Bacteroides koreensis, Bacteroides graminisolvens, Bacteroides uniformis, Bacteroides ovatus, Bacteroides xylanisolvens, Bacteroides stercoris, Bacteroides finegoldii, Bacteroides cellulosilyticus, Bacteroides kribbi, Bacteroides fragilis, Bacteroides massiliensis, Bacteroides dorei, Bacteroides faecis, Bacteroides salyersiae, or Bacteroides caccae cell.

42. The Bacteroides cell of claim 40 or claim 41, wherein the Bacteroides cell comprises one or more transgenes that allow for utilization of a privileged nutrient as a carbon source or increase its ability to utilize a privileged nutrient as a carbon source.

43. The Bacteroides cell of claim 42, wherein the privileged nutrient is a marine polysaccharide.

44. The Bacteroides cell of claim 43, wherein the marine polysaccharide is porphyran or agarose.

45. A method of treating a disease or disorder in a subject, the method comprising a step of administering to the subject a Bacteroides cell according to any one of claims 40-44.

46. The method of claim 45, wherein the disease or disorder is a gastrointestinal (GI) disease or disorder.

47. The method of claim 46, wherein the GI disease or disorder is selected from the group consisting of: irritable bowel syndrome (IBS), cancer (e.g., colorectal cancer), infectious colitis, ulcerative colitis, Crohn's disease, ischemic colitis, radiation colitis, peptic ulcer disease, gastritis, gastroenteritis, and celiac disease.

48. The method of any one of claims 45-47, wherein the subject is further administered porphyran.

49. The method of claim 48, wherein the porphyran is administered concurrently or sequentially with administration of the Bacteroides cell.

50. A method of producing a recombinant fusion polypeptide from an engineered Bacteroides, the method comprising culturing a Bacteroides cell comprising a polynucleotide of any one of claims 32-38, wherein the encoded recombinant fusion polypeptide is secreted outside of the Bacteroides cell.

51. The method of claim 50, further comprising collecting the recombinant fusion polypeptide.

52. A method of quantifying protein secretion from an engineered Bacteroides, the method comprising steps of:
(i) culturing a Bacteroides cell capable of expressing a recombinant fusion polypeptide according to any one of claims 1-31; and
(ii) measuring the amount of recombinant fusion polypeptide that is secreted outside of the Bacteroides cell.

53. The method of any one of claims 50-52, wherein at least 70%, at least 80%, at least 90%, at least 95%, or at least 99% of the recombinant fusion polypeptide is secreted as soluble protein.

54. The method of any one of claims 50-52, wherein the secreted protein is not associated with outer membrane vesicles (OMVs).

55. A method of quantifying protein secretion from an engineered Bacteroides, the method comprising steps of:
(i) administering to a subject a Bacteroides cell capable of expressing a recombinant fusion polypeptide according to any one of claims 1-31; and
(ii) measuring the amount of recombinant fusion polypeptide present in the feces of the subject, thereby to quantify the protein secretion from the engineered Bacteroides.

56. A method of assessing colonization by an engineered Bacteroides in the gut of a subject, the method comprising steps of:
(i) administering to the subject a Bacteroides cell capable of expressing a recombinant fusion polypeptide according to any one of claims 1-31; and
(ii) measuring the amount of recombinant fusion polypeptide present in the feces of the subject, thereby to assess colonization by the engineered Bacteroides in the gut of the subject.


SEQUENCE LISTING (73 sequences)

SEQ ID NO: 1 - DNA, 543 bp, synthetic construct
  source 1..543: mol_type = other DNA
atgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttgg

SEQ ID NO: 2 - protein, 181 aa, synthetic construct
MKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGW

SEQ ID NO: 3 - DNA, 483 bp, synthetic construct
  source 1..483: mol_type = other DNA
aatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttgg

SEQ ID NO: 4 - protein, 161 aa, synthetic construct
NDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGW

SEQ ID NO: 5 - DNA, 1104 bp, synthetic construct
  source 1..1104: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtctagtgtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcataa

SEQ ID NO: 6 - protein, 367 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGGSGGGSSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 7 - DNA, 744 bp, synthetic construct
  source 1..744: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaatggactacaaggatcatgatggggattacaaggaccatgacatagattacaaagatgatgatgataaagggtctagtgattacaaggacgatgatgataaggactacaaagatgatgatgacaaagattataaggacgatgatgataaaggtcatcatcatcatcatcactaa

SEQ ID NO: 8 - protein, 247 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGMDYKDHDGDYKDHDIDYKDDDDKGSSDYKDDDDKDYKDDDDKDYKDDDDKGHHHHHH

SEQ ID NO: 9 - DNA, 1140 bp, synthetic construct
  source 1..1140: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcaccgacagcttctcctctgggttccggtggctctggtggtgtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcataa

SEQ ID NO: 10 - protein, 379 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGGSGGGSPTASPLGSGGSGGVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 11 - DNA, 1125 bp, synthetic construct
  source 1..1125: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaggaggatccggcggagggtcaccgacagcttctcctctgggttccggtggctctggtggtgtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcataa

SEQ ID NO: 12 - protein, 374 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFEGGSGGGSPTASPLGSGGSGGVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 13 - DNA, 1116 bp, synthetic construct
  source 1..1116: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctggaggatccggcggagggtcaccgacagcttctcctctgggttccggtggctctggtggtgtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcataa

SEQ ID NO: 14 - protein, 371 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASGGSGGGSPTASPLGSGGSGGVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 15 - DNA, 1104 bp, synthetic construct
  source 1..1104: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattggggaggatccggcggagggtcaccgacagcttctcctctgggttccggtggctctggtggtgtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcataa

SEQ ID NO: 16 - protein, 367 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWGGSGGGSPTASPLGSGGSGGVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 17 - DNA, 6121 bp, synthetic construct
  source 1..6121: mol_type = other DNA
gataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtagcctggatctacttttaaaatttataaaatgttgaatttaaattatgtgaaaatgaagaaaatgagatttctattagtattattggtcgggatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcacgtgcgacggtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcaggttccgggtctggatcaggtagctaaggttcctaaggccatcctgacggatggccttttttttgactgctatgacttgataccggctattacgagcgcttaaacggcgcgcctgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcaggtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtcctttttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcactggtatctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgttctttacccgctgcttatctgcatcccatttggctacgtcaatccggtagcccgttgtaaactcgatacgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcgggtgtttttgttttatttaagataatacaaccactacataataaaagagtagcgatattaaaagaatccgatgagaaaagactaatatttatctatccattcagtttgatttctcaggactttacatcgtcctgaaagtatttgttgcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatggggcggccgcacgacgtaccggactcagtagggagagctgtatgtgggtag

SEQ ID NO: 18 - DNA, 1140 bp, synthetic construct; identical to SEQ ID NO: 9
  source 1..1140: mol_type = other DNA

SEQ ID NO: 19 - protein, 379 aa, synthetic construct; identical to SEQ ID NO: 10

SEQ ID NO: 20 - protein, 5 aa, synthetic construct
GGSGG

SEQ ID NO: 21 - protein, 5 aa, synthetic construct
GGGGS

SEQ ID NO: 22 - DNA, 744 bp, synthetic construct; identical to SEQ ID NO: 7
  source 1..744: mol_type = other DNA

SEQ ID NO: 23 - protein, 247 aa, synthetic construct; identical to SEQ ID NO: 8

SEQ ID NO: 24 - DNA, 852 bp, synthetic construct
  source 1..852: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaatggactacaaggatcatgatggggattacaaggaccatgacatagattacaaagatgatgatgataaagggtcaccatcggtccaagaagttgagaagctgcttcatgttcttgacagaaatggtgatggaaaggtgagtgcagaagaactgaaagcattcgcagatgactccaaatgtcctttggacagtaacaaaatcaaagcgttcattaaggaacatgacaagaacaaggacggaaaactggatctgaaggagctggttagtatccttagttcttaa

SEQ ID NO: 25 - protein, 283 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGMDYKDHDGDYKDHDIDYKDDDDKGSPSVQEVEKLLHVLDRNGDGKVSAEELKAFADDSKCPLDSNKIKAFIKEHDKNKDGKLDLKELVSILSS

SEQ ID NO: 26 - DNA, 1110 bp, synthetic construct
  source 1..1110: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaatggactacaaggatcatgatggggattacaaggaccatgacatagattacaaagatgatgatgataaagggtcacggccatctggaaaacgtccttgtaagatgcaggcatttcgtatttgggatacgaaccaaaagacattctaccttcgtaacaaccaattgatcgctggttatctgcaaggtcctaacatcaaattggaggaaaaaatcgacatggtgcctattgatttgcacagcgtattccttggcatacatggtggcaaactgtgtttatcatgcgcaaagtcgggagatgatatcaaacttcagctggaagaagtaaatataacagacctgagcaaaaacaaggaagaagataaacgctttacctttatacgttcagaaaaaggacctaccacatctttcgagtcagctgcatgtccgggatggtttctttgcacaactttggaggctgatcgcccggtgtctttaacaaacacgcccgaagagccacttatcgtaacgaaattttacttccaggaagaccaaggttcctaa

SEQ ID NO: 27 - protein, 369 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGMDYKDHDGDYKDHDIDYKDDDDKGSRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQGS

SEQ ID NO: 28 - DNA, 843 bp, synthetic construct
  source 1..843: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtctggaggagtggataataagttcaataaagaattaggttgggcaattggagaaattggcaccctgccaaacctgaatcatcagcaatttagagcctttatcctgagtttatgggatgatcctagtcagagtgcaaatttgcttgcagaggccaaaaaactgaatgatgcccaagcccctaaaggttccgactacaaggatcatgatggggattacaaggaccatgacatagattacaaagatgatgatgataaataa

SEQ ID NO: 29 - protein, 280 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGGSGGGSGGVDNKFNKELGWAIGEIGTLPNLNHQQFRAFILSLWDDPSQSANLLAEAKKLNDAQAPKGSDYKDHDGDYKDHDIDYKDDDDK

SEQ ID NO: 30 - DNA, 972 bp, synthetic construct
  source 1..972: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaatggactacaaggatcatgatggggattacaaggaccatgacatagattacaaagatgatgatgataaagggtcatctgacaaaataattcaccttacggatgacagctttgatacggacgtattgaaggctgatggagccatacttgtggatttttgggctgagtggtgtggtccgtgtaaaatgattgctccaatattggatgagattgctgatgaatatcaaggaaaacttactgtggccaaattgaatattgatcagaatccaggaacagccccaaaatatggcattcgcggtattccgaccttgttgctgtttaaaaatggagaggtggctgcaaccaaagtaggagctttatctaaaggccagttgaaagaattcctggatgcaaatttagcttaa

SEQ ID NO: 31 - protein, 323 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGMDYKDHDGDYKDHDIDYKDDDDKGSSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 32 - DNA, 1116 bp, synthetic construct
  source 1..1116: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaatggactacaaggatcatgatggggattacaaggaccatgacatagattacaaagatgatgatgataaagggtcactgaagcaagtagaaatatttacagatggttcctgtctgggaaatcctggaccgggaggttatggcgctatattacgctatcgcggccgtgaaaaaacattttccgctggttatacgcgtacaaccaacaatcgcatggaattgatggctgcaatcgtggcactggaggcactgaaagagcattgtgaagtcatcttaagcacggacagccaatacgtacgtcagggaataacgcaatggatccacaattggaagaaacgtggctggaagacggcagataaaaaacccgtgaagaacgtagatttatggcaacgtttagatgctgctctggggcaacatcaaatcaaatgggaatgggtaaaaggacatgctggacacccggaaaacgaaagatgcgatgagcttgcgagagctgcagcaatgaacccgaccttggaagacactggatatcaggttgaggtcggttcctaa

SEQ ID NO: 33 - protein, 371 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGMDYKDHDGDYKDHDIDYKDDDDKGSLKQVEIFTDGSCLGNPGPGGYGAILRYRGREKTFSAGYTRTTNNRMELMAAIVALEALKEHCEVILSTDSQYVRQGITQWIHNWKKRGWKTADKKPVKNVDLWQRLDAALGQHQIKWEWVKGHAGHPENERCDELARAAAMNPTLEDTGYQVEVGS

SEQ ID NO: 34 - DNA, 1026 bp, synthetic construct
  source 1..1026: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcagtccagttagttgagagcggcggtggactggtgcaagcaggtggcagcttgagcctgtcctgtagtgcctctggtcgttctttatctaattactatatgggctggttcagacaggctcctgggaaggagagagagctgttaggcaatattagctggcgtggttacaacatttattacaaggacagtgtgaaaggtcgctttacgatttcgcgggacgatgctaagaacaccatttacttacagatgaatagactgaagcctgaagacactgcagtgtattactgtgctgccagtattttgcctttgtccgacgatcctggctggaacacatattggggtcagggtacccaggtcacagtgtctggttccgactacaaggatcatgatggggattacaaggaccatgacatagattacaaagatgatgatgataaataa

SEQ ID NO: 35 - protein, 341 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGGSGGGSVQLVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVSGSDYKDHDGDYKDHDIDYKDDDDK

SEQ ID NO: 36 - DNA, 714 bp, synthetic construct
  source 1..714: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcagtaggtaacactttcttgcagtctactatcaaccgtactatcggtgtattgggttccgactacaaggatcatgatggggattacaaggaccatgacatagattacaaagatgatgatgataaataa

SEQ ID NO: 37 - protein, 237 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGGSGGGSVGNTFLQSTINRTIGVLGSDYKDHDGDYKDHDIDYKDDDDK

SEQ ID NO: 38 - DNA, 684 bp, synthetic construct
  source 1..684: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcaatgtcttactctgctggtttcggttccgactacaaggatcatgatggggattacaaggaccatgacatagattacaaagatgatgatgataaataa

SEQ ID NO: 39 - protein, 227 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGGSGGGSMSYSAGFGSDYKDHDGDYKDHDIDYKDDDDK

SEQ ID NO: 40 - DNA, 1944 bp, synthetic construct
  source 1..1944: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcagctacaaaaaaattacatttcgaaacattacaacttcacgtaggacaggaacagccagacccggctacggatgctcgtgcagttcctatttatcagactacatcttatgtatttcataactcggcacatgcagccgcacgttttggtttacaagatccgggtaatatttatgggcgtctgactaattccacacaaggtgtttttgaacaacgtgtggctgcattggaagggggagtggcaggacttgcggtagcttccggggctgctgccattacttatgcgtttcagaacattactcgtgcaggtgatcatgttgtagctgcaaagactatttacggaggtacttataacctgctggcgcatacgttgccgacttatggtgtgactgctacatttgtggaccctggtgatctttctaattttgagaaagctattcaggagaatacaaaggcggtgtttatcgagactttggggaatcctaattcgaatattatagatatagaggctgtggcggaaatagcacatcgccatcacatacctttgattattgataacacttttggtactccttatctgattcgtcccatcgagcacggagcagatattgtggtacattccgctacaaaatttattggtggccatggaacttcactgggtggcgtgattgttgattcaggtaaatttgactgggtggcatcaggtaaattccctcaactgacagaatcggatccttgctatcatggggtgcgttttgtagaggcggcaggtccggctgcttatgctgtccgtattcgtgctattttattgcgtgataccggtgcaactatcagtccgtttaatgcttttattctgttgcagggattggagacactttcattgcgtgtggaacgtcatgtagagaatgcgttgaaagtggtggagcatttgaaaaatcatccgaagatcaagaaagtaaatcacccttccttgccggaacatcctgaccatgctctgtatcagcgttatttccccaatggagctagttctattttcactatcgaagtgaaaggtggtcaggaagaagcacatcgtttcatcgatagtttggaaatcttttctttattggccaatgtcgccgatgtgaagtcattggttatccatcctgccagtaccactcactcccagttgaatgcggaagaattggcggaacagggtatttatccggggacagtgcgtttatcaataggtacggaacatatagatgatttgctggccgatttggatcaggcattagctggaattggttccgactacaaggatcatgatggggattacaaggaccatgacatagattacaaagatgatgatgataaataa

SEQ ID NO: 41 - protein, 647 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGWGGSGGGSATKKLHFETLQLHVGQEQPDPATDARAVPIYQTTSYVFHNSAHAAARFGLQDPGNIYGRLTNSTQGVFEQRVAALEGGVAGLAVASGAAAITYAFQNITRAGDHVVAAKTIYGGTYNLLAHTLPTYGVTATFVDPGDLSNFEKAIQENTKAVFIETLGNPNSNIIDIEAVAEIAHRHHIPLIIDNTFGTPYLIRPIEHGADIVVHSATKFIGGHGTSLGGVIVDSGKFDWVASGKFPQLTESDPCYHGVRFVEAAGPAAYAVRIRAILLRDTGATISPFNAFILLQGLETLSLRVERHVENALKVVEHLKNHPKIKKVNHPSLPEHPDHALYQRYFPNGASSIFTIEVKGGQEEAHRFIDSLEIFSLLANVADVKSLVIHPASTTHSQLNAEELAEQGIYPGTVRLSIGTEHIDDLLADLDQALAGIGSDYKDHDGDYKDHDIDYKDDDDK

SEQ ID NO: 42 - DNA, 6149 bp, synthetic construct
  source 1..6149: mol_type = other DNA
gataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtagcctggatcacacaacatttaaaaaataacattatgaaagcactcgaaaagagattcaaaggtaatattaacaataatttattttaatgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtctagtgtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcataaggttcctaaggccatcctgacggatggccttttttttgactgctatgacttgataccggctattacgagcgcttaaacggcgcgcctgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcaggtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtcctttttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcactggtatctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgttctttacccgctgcttatctgcatcccatttggctacgtcaatccggtagcccgttgtaaactcgatacgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcgggtgtttttgttttatttaagataatacaaccactacataataaaagagtagcgatattaaaagaatccgatgagaaaagactaatatttatctatccattcagtttgatttctcaggactttacatcgtcctgaaagtatttgttgcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatggggcggccgcacgacgtaccggactcagtagggagagctgtatgtgggtag

SEQ ID NO: 43 - DNA, 6120 bp, synthetic construct
  source 1..6120: mol_type = other DNA
gataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtagcctggatcacacaacatttaaaaaataacattatgaaagcactcgaaaagagattcaaaggtaatattaacaataatttattttaatgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcacgtgcgacggtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcaggttccgggtctggatcaggtagctaaggttcctaaggccatcctgacggatggccttttttttgactgctatgacttgataccggctattacgagcgcttaaacggcgcgcctgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcaggtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtcctttttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcactggtatctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgttctttacccgctgcttatctgcatcccatttggctacgtcaatccggtagcccgttgtaaactcgatacgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcgggtgtttttgttttatttaagataatacaaccactacataataaaagagtagcgatattaaaagaatccgatgagaaaagactaatatttatctatccattcagtttgatttctcaggactttacatcgtcctgaaagtatttgttgcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacgg

SEQ ID NO: 44 - DNA, 6121 bp, synthetic construct
  source 1..6121: mol_type = other DNA
gataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtagcctgcgattatctttaaattttataaaatgttaaatttaaattacgttaagatgaagaaaatgagattcttattggttttattggtggggatatcgattgtctcttgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcacgtgcgacggtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcaggttccgggtctggatcaggtagctaaggttcctaaggccatcctgacggatggccttttttttgactgctatgacttgataccggctattacgagcgcttaaacggcgcgcctgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcaggtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtcctttttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcactggtatctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgttctttacccgctgcttatctgcatcccatttggctacgtcaatccggtagcccgttgtaaactcgatacgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcgggtgtttttgttttatttaagataatacaaccactacataataaaagagtagcgatattaaaagaatccgatgagaaaagactaatatttatctatccattcagtttgatttctcaggactttacatcgtcctgaaagtatttgttgcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatggggcggccgcacgacgtaccggactcagtagggagagctgtatgtgggtag

SEQ ID NO: 45 - DNA, 6121 bp, synthetic construct; identical to SEQ ID NO: 17
  source 1..6121: mol_type = other DNA

SEQ ID NO: 46 - DNA, 6106 bp, synthetic construct
  source 1..6106: mol_type = other DNA
gataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtagcctgcgattatctttaaattttataaaatgttaaatttaaattacgttaagatgaagaaaatgagattcttattggttttattggtggggatatcgattgtctcttgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcacgtgcgacggtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcaccgcagccgtaaggttcctaaggccatcctgacggatggccttttttttgactgctatgacttgataccggctattacgagcgcttaaacggcgcgcctgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcaggtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtcctttttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcactggtatctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgttctttacccgctgcttatctgcatcccatttggctacgtcaatccggtagcccgttgtaaactcgatacgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcgggtgtttttgttttatttaagataatacaaccactacataataaaagagtagcgatattaaaagaatccgatgagaaaagactaatatttatctatccattcagtttgatttctcaggactttacatcgtcctgaaagtatttgttgcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatggggcggccgcacgacgtaccggactcagtagggagagctgtatgtgggtag

SEQ ID NO: 47 - DNA, 6106 bp, synthetic construct
  source 1..6106: mol_type = other DNA
gataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtagcctgcgattatctttaaattttataaaatgttaaatttaaattacgttaagatgaagaaaatgagattcttattggttttattggtggggatatcgattgtctcttgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttggggaggatccggcggagggtcacgtgcgacggtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcaaattcgcagtaaggttcctaaggccatcctgacggatggccttttttttgactgctatgacttgataccggctattacgagcgcttaaacggcgcgcctgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcaggtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtcctttttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcactggtatctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgttctttacccgctgcttatctgcatcccatttggctacgtcaatccggtagcccgttgtaaactcgatacgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcgggtgtttttgttttatttaagataatacaaccactacataataaaagagtagcgatattaaaagaatccgatgagaaaagactaatatttatctatccattcagtttgatttctcaggactttacatcgtcctgaaagtatttgttgcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatggggcggccgcacgacgtaccggactcagtagggagagctgtatgtgggtag

SEQ ID NO: 48 - DNA, 174 bp, synthetic construct
  source 1..174: mol_type = other DNA
ggaatggatacgtataaactgatactgaacggaaaaaccctgaaaggagaaacgaccactgaagctgtagatgcagcgactgctgaaaaagtcttcaaacagtacgctaatgataatggtgtcgacggagaatggacctatgatgatgcaactaaaacatttaccgtgacggaa

SEQ ID NO: 49 - protein, 58 aa, synthetic construct
GMDTYKLILNGKTLKGETTTEAVDAATAEKVFKQYANDNGVDGEWTYDDATKTFTVTE

SEQ ID NO: 50 - DNA, 174 bp, synthetic construct
  source 1..174: mol_type = other DNA
gtggataataagttcaataaagaattaggttgggcaattggagaaattggcaccctgccaaacctgaatcatcagcaatttagagcctttatcctgagtttatgggatgatcctagtcagagtgcaaatttgcttgcagaggccaaaaaactgaatgatgcccaagcccctaaa

SEQ ID NO: 51 - protein, 58 aa, synthetic construct
VDNKFNKELGWAIGEIGTLPNLNHQQFRAFILSLWDDPSQSANLLAEAKKLNDAQAPK

SEQ ID NO: 52 - DNA, 1281 bp, synthetic construct
  source 1..1281: mol_type = other DNA
gctacaaaaaaattacatttcgaaacattacaacttcacgtaggacaggaacagccagacccggctacggatgctcgtgcagttcctatttatcagactacatcttatgtatttcataactcggcacatgcagccgcacgttttggtttacaagatccgggtaatatttatgggcgtctgactaattccacacaaggtgtttttgaacaacgtgtggctgcattggaagggggagtggcaggacttgcggtagcttccggggctgctgccattacttatgcgtttcagaacattactcgtgcaggtgatcatgttgtagctgcaaagactatttacggaggtacttataacctgctggcgcatacgttgccgacttatggtgtgactgctacatttgtggaccctggtgatctttctaattttgagaaagctattcaggagaatacaaaggcggtgtttatcgagactttggggaatcctaattcgaatattatagatatagaggctgtggcggaaatagcacatcgccatcacatacctttgattattgataacacttttggtactccttatctgattcgtcccatcgagcacggagcagatattgtggtacattccgctacaaaatttattggtggccatggaacttcactgggtggcgtgattgttgattcaggtaaatttgactgggtggcatcaggtaaattccctcaactgacagaatcggatccttgctatcatggggtgcgttttgtagaggcggcaggtccggctgcttatgctgtccgtattcgtgctattttattgcgtgataccggtgcaactatcagtccgtttaatgcttttattctgttgcagggattggagacactttcattgcgtgtggaacgtcatgtagagaatgcgttgaaagtggtggagcatttgaaaaatcatccgaagatcaagaaagtaaatcacccttccttgccggaacatcctgaccatgctctgtatcagcgttatttccccaatggagctagttctattttcactatcgaagtgaaaggtggtcaggaagaagcacatcgtttcatcgatagtttggaaatcttttctttattggccaatgtcgccgatgtgaagtcattggttatccatcctgccagtaccactcactcccagttgaatgcggaagaattggcggaacagggtatttatccggggacagtgcgtttatcaataggtacggaacatatagatgatttgctggccgatttggatcaggcattagctggaatt

SEQ ID NO: 53 - protein, 427 aa, synthetic construct
ATKKLHFETLQLHVGQEQPDPATDARAVPIYQTTSYVFHNSAHAAARFGLQDPGNIYGRLTNSTQGVFEQRVAALEGGVAGLAVASGAAAITYAFQNITRAGDHVVAAKTIYGGTYNLLAHTLPTYGVTATFVDPGDLSNFEKAIQENTKAVFIETLGNPNSNIIDIEAVAEIAHRHHIPLIIDNTFGTPYLIRPIEHGADIVVHSATKFIGGHGTSLGGVIVDSGKFDWVASGKFPQLTESDPCYHGVRFVEAAGPAAYAVRIRAILLRDTGATISPFNAFILLQGLETLSLRVERHVENALKVVEHLKNHPKIKKVNHPSLPEHPDHALYQRYFPNGASSIFTIEVKGGQEEAHRFIDSLEIFSLLANVADVKSLVIHPASTTHSQLNAEELAEQGIYPGTVRLSIGTEHIDDLLADLDQALAGI

SEQ ID NO: 54 - DNA, 51 bp, synthetic construct
  source 1..51: mol_type = other DNA
gtaggtaacactttcttgcagtctactatcaaccgtactatcggtgtattg

SEQ ID NO: 55 - protein, 17 aa, synthetic construct
VGNTFLQSTINRTIGVL

SEQ ID NO: 56 - DNA, 21 bp, synthetic construct
  source 1..21: mol_type = other DNA
atgtcttactctgctggtttc

SEQ ID NO: 57 - protein, 7 aa, synthetic construct
MSYSAGF

SEQ ID NO: 58 - DNA, 456 bp, synthetic construct
  source 1..456: mol_type = other DNA
cggccatctggaaaacgtccttgtaagatgcaggcatttcgtatttgggatacgaaccaaaagacattctaccttcgtaacaaccaattgatcgctggttatctgcaaggtcctaacatcaaattggaggaaaaaatcgacatggtgcctattgatttgcacagcgtattccttggcatacatggtggcaaactgtgtttatcatgcgcaaagtcgggagatgatatcaaacttcagctggaagaagtaaatataacagacctgagcaaaaacaaggaagaagataaacgctttacctttatacgttcagaaaaaggacctaccacatctttcgagtcagctgcatgtccgggatggtttctttgcacaactttggaggctgatcgcccggtgtctttaacaaacacgcccgaagagccacttatcgtaacgaaattttacttccaggaagaccaa

SEQ ID NO: 59 - protein, 152 aa, synthetic construct
RPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 60 - DNA, 363 bp, synthetic construct
  source 1..363: mol_type = other DNA
gtccagttagttgagagcggcggtggactggtgcaagcaggtggcagcttgagcctgtcctgtagtgcctctggtcgttctttatctaattactatatgggctggttcagacaggctcctgggaaggagagagagctgttaggcaatattagctggcgtggttacaacatttattacaaggacagtgtgaaaggtcgctttacgatttcgcgggacgatgctaagaacaccatttacttacagatgaatagactgaagcctgaagacactgcagtgtattactgtgctgccagtattttgcctttgtccgacgatcctggctggaacacatattggggtcagggtacccaggtcacagtgtct

SEQ ID NO: 61 - protein, 121 aa, synthetic construct
VQLVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVS

SEQ ID NO: 62 - DNA, 18 bp, synthetic construct
  source 1..18: mol_type = other DNA
ggttccgggtctggatca

SEQ ID NO: 63 - protein, 8 aa, synthetic construct
GSGSGSGS

SEQ ID NO: 64 - DNA, 18 bp, synthetic construct
  source 1..18: mol_type = other DNA
cctacagcgtcccctctc

SEQ ID NO: 65 - protein, 6 aa, synthetic construct
PTASPL

SEQ ID NO: 66 - DNA, 567 bp, synthetic construct
  source 1..567: mol_type = other DNA
atgttaaatttaaattatgtaaagatgaaaaaaatgagatttttattagtattattagtgggtatatctattgtatcatgtactaatgacttagatgaaatgctagggactcaaaatttgcaaacccgttctgttgagcctagattcacaggaaatatatcagaaagtcaggcgattgcattaatgaataaatttctggatgcaccctcttttttttattttgaagatgtcataggcttcgatccgacaacatctatttccacaggaagtttcactgtcaacagaaatgggaaaactatacatgtacgttctattcaagttggtgtaataggaaagaacggaaaatatggagtttatggaactccagtaacgagcaaagaattctattcattgagaaatgggatggttttgcatggagaacctatttatgaagagaatgatgtagaaagagccaacccagtaggaatgcgttattactatacaactaatgatgtaaatcatagtcaaaattgctggattgcgatcaattgggctgatgcttctacatttgaaagtataattggttgg

SEQ ID NO: 67 - protein, 189 aa, synthetic construct
MLNLNYVKMKKMRFLLVLLVGISIVSCTNDLDEMLGTQNLQTRSVEPRFTGNISESQAIALMNKFLDAPSFFYFEDVIGFDPTTSISTGSFTVNRNGKTIHVRSIQVGVIGKNGKYGVYGTPVTSKEFYSLRNGMVLHGEPIYEENDVERANPVGMRYYYTTNDVNHSQNCWIAINWADASTFESIIGW

SEQ ID NO: 68 - protein, 6 aa, synthetic construct
HHHHHH

SEQ ID NO: 69 - protein, 8 aa, synthetic construct
GSGSGSGS

SEQ ID NO: 70 - protein, 20 aa, synthetic construct
MKKMRFLLVLLVGISIVSCT

SEQ ID NO: 71 - protein, 28 aa, synthetic construct
MLNLNYVKMKKMRFLLVLLVGISIVSCT

SEQ ID NO: 72 - protein, 11 aa, synthetic construct
SIVSCTNDLDE

SEQ ID NO: 73 - protein, 8 aa, synthetic construct
DYKDDDDK